(19) United States
(12) Patent Application Publication
(10) Pub. No.: US 2007/0154889 A1
(43) Pub. Date: Jul. 5, 2007

(54) Methods and reagents for the detection of melanoma

(75) Inventor: Yixin Wang, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: VERIDEX, LLC, Warren, NJ (US)

(21) Appl. No.: 10/567,025

(22) PCT Filed: Jun. 24, 2005
(86) PCT No.: PCT/US05/22846

§ 371 Date: Aug. 16, 2006

Related U.S. Application Data
(60) Provisional application No. 60/582,906, filed on Jun. 25, 2004.
(60) Provisional application No. 60/612,951, filed on Sep. 24, 2004.
(60) Provisional application No. 60/649,725, filed on Feb. 3, 2005.

(51) Int. Cl. C12Q 1/68; C12P 19/34

(52) U.S. Cl. 435/6; 435/91.2

(57) ABSTRACT

An assay for identifying a malignant melanocyte is conducted by determining whether differential expression of particular genes is indicative of melanoma exceed a cut-off value. The assay can be performed intra-operatively on lymph node tissue.

### BACKGROUND OF THE INVENTION

Cutaneous malignant melanoma is a common, aggressive cancer with growing incidence. It is a serious healthcare problem with over 55,100 new cases anticipated in 2004 in US, and a mortality rate of about 14.5%. Cancer Facts and Figures 2003. American Cancer Society, 2003. The incidence of melanoma continues to rise faster than that of any other malignancy. De Braud et al. (2003). While prognosis of early local melanoma is favorable with 5-year overall survival over 90%, regional lymph node involvement decreases the overall survival rate to 10-46%. Balch et al. (2001). Therefore regional lymph node (LN) status becomes the most significant prognostic factor in a melanoma patient's survival. Introduction of the sentinel lymph nodes (SLN) technique (Morton (1992)) has increased the sensitivity of melanoma micrometastasis detection compared to H&E staining alone. Yu et al. (1999); and Messina et al. (1999). Nevertheless, even when enhanced by IHC, histological analysis is limited by the ability of light microscopy to recognize the tumor cells. Reverse transcription-polymerase chain reaction (RT-PCR) analysis has recently been proposed for a more sensitive detection of melanoma cells in LN. Many studies, when using well-characterized melanocyte specific markers, such as tyrosinase and MART-I, h [symbol] /e demonstrated the presence of these gene transcripts in LNs otherwise found to be negative by routine histology and IHC. Shivers et al. (1998); and Kuo et al. (2003). However, these genes are not specific to tumor cells and cannot be used to discriminate between benign and malignant tissue. hi fact, they caused false-positive results in the presence of benign capsular nevi. Takeuchi et al. (2004); Starz et al. (2003); and Gutzmer et al. (2002). Considering that benign nevi are not rare events in the melanoma SLN, the current RT-PCR assays are not useful clinically for diagnostic of melanoma micrometastasis. A recent study, proposed a multi-marker panel, including cancer specific markers for RT-PCR assay in order to increase assay specificity. Hoon et al. (2004). Identification of novel melanoma specific markers remains one of the key questions of melanoma research.

Certain proteins have been shown to be associated with melanoma and its metastases. These proteins or their activities have been used in IHC to identify metastases and include LlCAM (Thies et al. (2002); Fogel et al. (2003)); and S-100 (Diego et al. (2003)).

Nucleic acid tests have been proposed to increase the sensitivity of detection of metastatic melanoma. US Patent Publication Nos. 2002/01 10820; and 2003/0232356. Studies have used markers that include MAGE3, tyrosinase, MART-1, MITF-M or IL-I, R1, endothelin-2, ephrin-A5, IGF Binding protein 7, HLA-A0202 heavy chain, Activin A (βA subunit), TNF RII, SPC4, CNTF Ra, or gplOO (HMB45) genes. Bostick et al. (1999); Hoon et al. (2001); Palmieri et al. (2001); Wrightson et al. (2001); Gutzmer et al. (2002); Davids et al. (2003); Starz et al. (2003); Rimboldi et al. (2003); Cook et al (2003); Reintgen et al. (2004); US Patent Publication Nos. 2002/0098535; 2003/0049701; U.S. Pat. Nos. 5,512,437; 5,512,444; 5,612,201; 5,759,783; 5,844,075; 6,025,474; 6,057,105; 6,235,525; 6,291,430; 6,338,947; 6,369,211; 6,426,217; 6,475,727; 6,500,919; 6,527,560; 6,599,699; WO 96/29430. Where determined, these markers have not been found adequate for sole use in melanoma diagnosis. Riccioni et al. (2002); Gutzmer et al. (2002); Davids et al. (2003); Goydos et al. (2003); and Prichard et al. (2003).

A number of these markers have also been shown to be indicative of other neoplasias such as ME20M (GPlOO) for clear cell sarcoma, biliary tract carcinoma and gastric carcinoma. Hiraga et al. (1997); Okada et al. (2001); Okami et al. (2001); Antonescu et al. (2002); Segal et al. (2003). MAGE3 is also indicative of a number of neoplasias including breast, hepatocellular, renal, neural, lung and esophageal. Yamanaka et al. (1999); Ooka et al. (2000); Suzuki et al. (2000); Cheung et al. (2001); and Weiser et al. (2001). Several melanoma antigen-encoding genes are also expressed in lung cancer. Yoshimatsu et al. (1998).

These markers proved to be sensitive but non-specific since they showed positive expression in other cancers and benign melanocytes. Additionally, tyrosinase is expressed in Schwann cells which are present in normal lymph nodes. The lack of specificity alone calls for the development of assays with new or additional markers. H&E histology and IHC remain the “gold standard for the identification of melanoma and nevus cells in SLNs.” Starz et al. (2003). Detection issues in the intra-operative setting make this need even more acute.

Lymph node involvement is the strongest prognostic factor in many solid tumors, and detection of lymph node micrometastases is of great interest to pathologists and surgeons. Current lymph node evaluation involves microscopic examination of H&E-stained tissue sections and IHC and suffers from three major limitations: (a) small foci of cells, are easily missed; (b) the result is not rapidly available, meaning that any positive result in a SLN procedure requires a second surgery for removal of axcillary lymph nodes; and (c) only one or two tissue sections are studied, and thus the vast majority of each node is left unexamined. Serial sectioning can help overcome sampling error, and IHC can help identify small foci of cells; this combination, however, is costly and time consuming for routine analysis.

Surgical decisions of regional lymph node dissection can be based on intra-operative frozen section analysis of lymph nodes; however, the sensitivity of these methods is relatively poor, ranging from 50-70% relative to standard H&E pathology, leading to a high rate of second surgeries. Thus, pathologists are not routinely performing intra-operative frozen section analysis or touch print cytology analysis for melanoma patients. Improvements in the sensitivity and specificity of intra-operative assays for melanoma would significantly benefit oncology.

High-density microarrays have been applied to simultaneously monitor expression, in biological samples, of thousands of genes. Studies have resulted in the identification of genes differentially expressed in benign and malignant lesions, as well as genes that might be of prognostic value. Luo et al. (2001); and Wang et al. (2004). Gene expression profiling of malignant melanoma has been accomplished using a microarray containing probes for 8,150 cDNAs. Bittner et al. (2000). These researchers identified several genes that might be associated with aggressive tumor behavior. In recent work, comparison of gene expression profiles of a few melanoma and normal melanocyte cell lines led to the identification of differentially expressed genes and pathways modulated in melanoma. Takeuchi et al. (2004).

### SUMMARY OF THE INVENTION

Gene expression profiling of an extensive set of clinically relevant tissue samples is provided in the present invention. Total RNA from forty-five primary malignant melanomas, 18 benign skin nevi and 7 normal skin tissues were hybridized on an Affymetrix HuI 33A microarray containing 22,000 probe sets. Differentially expressed genes in malignant melanoma as compared to benign tissue were identified. Pathway analysis of the differentially expressed genes revealed an over-representation of genes associated with neural tissue development and activation of amyloid processing signaling pathway. A one-step quantitative RT-PCR assay was used to test a combination of two melanoma specific genes, PLAB and LlCAM in a panel of clinically relevant samples that included primary malignant melanoma, benign nevi, melanoma LN metastasis and melanoma-free lymph node samples.

The present invention provides a method of identifying a melanoma by obtaining a tissue sample; and assaying and measuring the expression levels in the sample of genes encoding mRNA corresponding to prostate differentiation factor (PLAB, MICl) (SEQ ID NO: 1) and Ll cell adhesion molecule (LlCAM) (SEQ ID NO: 2); or PLAB, LlCAM and neurotrophic tyrosine kinase receptor, type 3 (NTRK3) (SEQ ID NO: 3) where the gene expression levels above pre-determined cut-off levels are indicative of the presence of a melanoma in the sample. The invention farther provides a method of identifying a melanoma by obtaining a tissue sample; and assaying and measuring the expression levels in the sample of genes encoding mRNA recognized by the primer/probe sets SEQ ID NOs: 4-6 or SEQ ID NOs: 7-9 and SEQ ID NOs: 10-12 or SEQ ID NOs: 13-15; or SEQ ID NOs: 4-6 or SEQ ID NOs: 7-9 and SEQ ID NOs: 10-12 or SEQ ID NOs: 13-15 and SEQ ID NOs: 16-18 where the gene expression levels above pre-determined cut-off levels are indicative of the presence of a melanoma in the sample.

The invention also provides a method of distinguishing a malignant melanocyte from a benign melanocyte by obtaining a tissue sample; and assaying and measuring the expression levels in the sample of genes encoding PLAB and LlCAM; or PLAB, LlCAM and NTRK3 where the gene expression levels above pre-determined cut-off levels are indicative of the presence of a melanoma in the sample.

The invention also provides a method of distinguishing a malignant melanocyte from a benign melanocyte by obtaining a tissue sample; and assaying and measuring the expression levels in the sample of genes recognized by the primer/probe sets SEQ ID NOs: 4-6 or SEQ ID NOs: 7-9 and SEQ ID NOs: 10-12 or SEQ ID NOs: 13-15; or SEQ ID NOs: 4-6 or SEQ ID NOs: 7-9 and SEQ ID NOs: 10-12 or SEQ ID NOs: 13-15 and SEQ ID NOs: 16-18 where the gene expression levels above pre-determined cut-off levels are indicative of the presence of a melanoma in the sample.

The invention further provides a method of determining patient treatment protocol by obtaining a tissue sample from the patient; and assaying and measuring the expression levels in the sample of genes encoding PLAB and LlCAM; or PLAB, LlCAM and NTRK3 where the gene expression levels above pre-determined cut-off levels are indicative of the presence of a melanoma in the sample.

The invention further provides a method of determining patient treatment protocol by obtaining a tissue sample from the patient; and assaying and measuring the expression levels in the sample of genes recognized by the primer/probe sets SEQ ID NOs: 4-6 or SEQ ID NOs: 7-9 and SEQ ID NOs: 10-12 or SEQ ID NOs: 13-15; or SEQ ID NOs: 4-6 or SEQ ID NOs: 7-9 and SEQ ID NOs: 10-12 or SEQ ID NOs: 13-15 and SEQ ID NOs: 16-18 where the gene expression levels above pre-determined cut-off levels are indicative of the presence of a melanoma in the sample.

The invention further provides additional Marker and control genes, the expression of which aid in the claimed methods. These additional genes include up-regulated SEQ ID NOs: 29-467 and down-regulated SEQ ID NOs: 468-978.

The primary Marker can be PLAB and is defined herein as the gene encoding any variant, allele etc. including SEQ ID NO: 1. PLAB is also described by Paralkar et al. (1998) and represented by Accession No. AF003934. PLAB is also defined as the gene encoding mRNA recognized by the primer/probe sets SEQ ID NOs: 4-9.

The secondary Marker can be LlCAM and is defined herein as the gene encoding any variant, allele etc. including SEQ ID NO: 2. LlCAM is also described by Haspel et al (2003); and U.S. Pat. No. 6,107,476 and is represented by Accession No. NM_{—}000425. LlCAM is also defined as the gene encoding mRNA recognized by the primer/probe sets SEQ ID NOs: 10-15.

The invention further provides a kit for conducting an assay to determine the presence of melanoma in a cell sample comprising: nucleic acid amplification and detection reagents.

The invention further provides primer/probe sets for amplification and detection of PCR products obtained in the inventive methods. These sets include the following:

SEQ ID NO:4 (PLAB forward primer)
ggcagaatcttcgtccgca
SEQ ID NO:5 (PLAB reverse primer)
ggacagtggtccccgttg
SEQ ID NO.6 (PLAB probe)
cccagctggagttgcacttgcggcc
SEQ ID NO:7 (PLAB upper primer)
gaacaccgacctcgtccc
SEQ ID NO:8 (PLAB lower primer)
ggcggcccgagagata
SEQ ID NO:9 (PLAB probe)
cgccagaagtgcggctgggattt
SEQ ID NO:10 (L1CAM forward)
gctgggactgggaacagaact
SEQ ID NO:11 (L1CAM Reverse)
ggagcagagatggcaaagaaa
SEQ ID NO:12 (L1CAM probe)
ttccccaccatctgctgt
SEQ ID NO:13 (L1CAM upper)
ccacagatgacatcagcctcaa
SEQ ID NO:14 (L1CAM lower)
ggtcacacccagctcttcctt
SEQ ID NO:15 (L1CAM probe)
tggcaagcccgaagtgcagttcctt
SEQ ID NO:16 (NTRK3 primer)
gccccggcacccttta
SEQ ID NO:17 (NTRK3 primer)
aaccctgccagtggtggat
SEQ ID NO:18 (NTRK3 probe)
cagatgggtgttttc
SEQ ID NO:19 (Tyr upper)
actcagcccagcatcattcttc
SEQ ID NO:20 (Tyr lower)
atggctgttgtactcctccaatc
SEQ ID NO:21 (Tyr probe)
cttctcctcttggcagattgtctgtagctt
SEQ ID NO:22 (PBGD upper)
ccacacacagcctactttccaa
SEQ ID NO:23 (PBGD lower)
tacccacgcgaatcactctca
SEQ ID NO:24 (PBGD probe)
aacggcaatgcggctgcaacggcggaatt

The invention further provides amplicons obtained by PCR methods utilized in the inventive methods. These amplicons include the following:

SEQ ID NO:25 (PLAB Amplicon)
gaacaccgacctcgtcccggcccctgcagtccggatactcacgccagaag
tgcggctgggatccggcggccacctgcacctgcgtatctctcgggccgcc
SEQ ID NO:26 (L1CAM Amplicon)
ccacagatgacatcagcctcaagtgtgaggccagtggcaagcccgaagtg
cagttccgctggacgagggatggtgtccacttcaaacccaaggaagagct
gggtgtgacc
SEQ ID NO:27 (tyrosinase Amplicon)
actcagcccagcatcattcttctcctcttggcagattgtctgtagccgat
tggaggagtacaacagccat
SEQ ID NO:28 (PBGD Amplicon)
ccacacacagcctactttccaagcggagccatgtctggtaacggcaatgc
ggctgcaacggcggaagaaaacagcccaaagatgagagtgattcgcgtgg
gta

Other genes described herein include up-regulated Markers (SEQ ID NOs: 29-467), down-regulated Markers (SEQ ID NOs: 468-978), PBGD (SEQ ID NO: 979), MARTl (SEQ ID NO: 980), ME20M (GPlOO; SEQ ID NO: 981) and MAGE-3 (SEQ ID NO: 982) and various primers and probes (SEQ ID NOs: 983-1011) used in detecting their expression.

### BRIEF DESCRIPTION OF THE DRAWINGS

FIG. 1. Flowchart of data analysis.

FIG. 2. Hierarchical clustering on the 15,795 genes that have at least two “present” calls in all samples. Each column is a sample and each row is a gene. Red is up-regulation and green is down-regulation. Purple: melanoma samples; yellow: benign nevi; and blue: normal skin.

FIG. 3. Microarray expression (A) and real time RT-PCR validation data (B) of the selected genes. First fourteen samples from the left are the melanoma tissue samples (red); next seven are benign nevi samples (yellow) and last five are normal skin (blue). For microarray plots x-axis shows intensity values; for PCR plots, x-axis is 2^{ΔCT}, where ΔCt is Ct (Target Gene)—Ct PBGD.

FIG. 4. Amyloid processing pathway. Adopted from Ingenuity™ Pathway Analysis Software Application. Genes up-regulated in melanoma are red and down-regulated in melanoma are green. Each gene symbol is followed by the fold-change of expression level between melanoma and benign/normal samples.

FIG. 5. One-step quantitative RT-PCR assay of PLAB and LlCAM (A) and conventional melanoma markers, gplOO, tyrosinase (SEQ ID NO: 999) and MARTl (B). For each plot x-axis represents score for the new markers or the conventional markers. Median scores for each samples category are labeled. Two cut-off levels based on normal (green) and benign (red) samples are labeled on each plot.

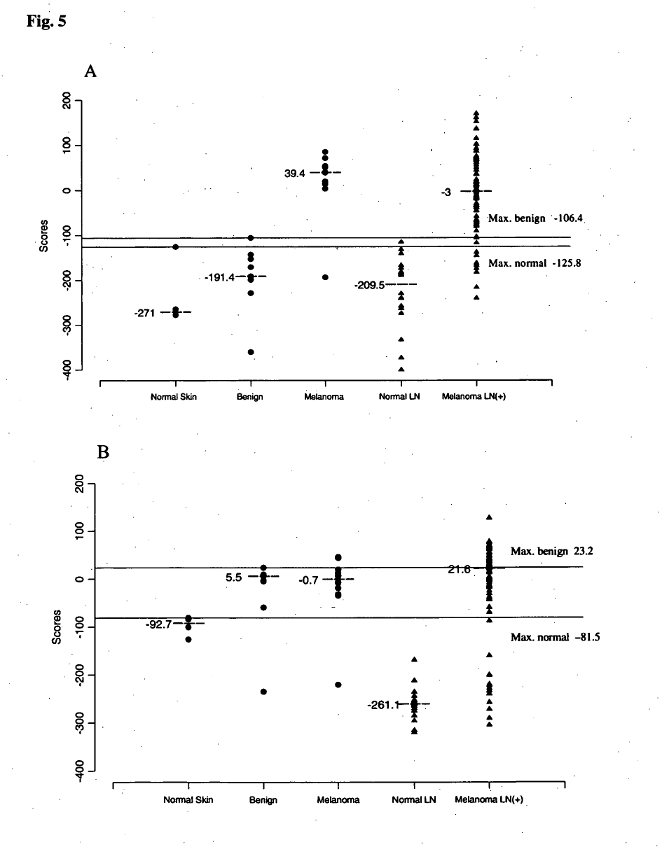

### DETAILED DESCRIPTION

The present invention provides methods of qualitatively and quantitatively identifying a melanoma; distinguishing a malignant melanocyte from a benign melanocyte; diagnosing melanocytic lesions with uncertain pathological features; and determining a melanoma patient treatment protocol. The methods further provide aids in patient prognosis, patient monitoring and drug development. The methods rely on assaying and measuring expression levels of various Marker genes encoding mRNAs provided herein where gene expression over a pre-determined cut-off level is indicative of the presence of a malignant melanocyte in the sample assayed.

Cutaneous melanoma is a common, aggressive cancer with growing incidence. Identification of melanoma-specific deregulated genes could provide molecular markers for LN staging assays and further insight on melanoma tumorigenesis. Total RNA isolated from 45 primary melanoma, 18 benign skin nevi, and 7 normal skin tissue specimens were analyzed on an Affymetrix U133A microarray containing 22,000 probe sets. Hierarchical clustering revealed a distinct separation of the melanoma samples from benign and normal specimens. Novel genes associated with malignant melanoma were identified. Differential gene expression of two melanoma specific genes, PLAB and LlCAM, were tested by a one-step quantitative RT-PCR assay on primary malignant melanoma, benign nevi and normal skin samples and also on malignant melanoma LN metastasis and melanoma-free lymph nodes. The performance of the markers was compared to conventional melanoma markers such as tyrosinase, gplOO, and MARTl. The results demonstrated the ability of using a combination of PLAB and LlCAM in a RT-PCR assay to differentiate clinically relevant tissue samples containing benign or malignant melanocytes.

High-density cDNA and oligonucleotide microarrays allow simultaneous monitoring of the expression of thousands of genes. Microarray technology provides a quantitative measurement of mRNA abundance and has gained acceptance as a tool for marker discovery based on gene expression. In the context of cancer research, microarray analysis has identified genes differentially expressed in benign and malignant lesions, different cancer types or that have prognostic significance. Luo et al. (2001); Su et al. (2001); Henshall et al. (2003); and Wang et al. (2004). The first gene expression profiling of malignant melanoma used a microarray containing probes for 8,150 cDNAs and identified genes that might be associated with aggressive tumor behavior. Bittner et al. (2000). Since the samples analyzed in their study did not include tissues containing normal or benign melanocytes, differentially expressed genes in malignant melanoma were not identified. hi contrast to normal skin, melanocyte content in benign nevi is close to that in melanoma.

In another study, two pooled samples derived from either melanoma or benign nevi tissues were hybridized to a cDNA array and genes preferentially expressed in melanoma- or nevi-derived samples were found. Seykora et al. (2003). Other researchers used subtractive hybridization or analysis of SAGE libraries generated on melanoma cell lines, for monitoring gene expression in melanoma. Hipfel et al. (2000); and Weeraratna (2004). Recently, comparison of gene expression profiles of a few melanoma and melanocyte cell lines led to the identification of differentially expressed genes and pathways modulated in melanoma. Hoek et al. (2004). While these studies provide a solid foundation for melanoma genetics, there is no marker that can clearly differentiate melanoma from benign tissue. Several markers currently used such as tyrosinase and Mart-1 cannot discriminate between benign and malignant tissue. Takeuchi et al. (2004). Consequently, these markers have limited use in applications such as intra-operative, lymph-node-based staging of disease.

Difficulties in obtaining sufficient RNA samples from malignant melanoma and benign melanocyte lesions, tissue heterogeneity, and the presence of melanin in purified RNA remain the major challenges in these studies. In the study presented herein, total RNA isolated from 45 primary malignant melanomas, 18 benign skin nevi, and 7 normal skin tissues were hybridized on an Affymetrix HuI 33A microarray containing 22,000 probe sets. A modified RNA extraction method was developed to produce melanin-free RNA samples that increased the micorarray hybridization signals. Hierarchical clustering revealed distinct separation of the melanoma samples from benign and normal specimens. Significance Analysis of Microarray (SAM) method, t-test and percentile analysis identified 439 up-regulated (SEQ ID NOs: 29-467) and 511 down-regulated (SEQ ID NOs: 468-978) genes in the melanoma samples. Besides well-characterized genes such as me20m (gplOO), melanocortin receptor 1, and LlCAM, many novel genes previously unassociated with melanoma were identified including NTKR3 and PLAB. Pathway analysis of the differentially expressed genes revealed an over-representation of genes associated with neural tissue development and function, activation of amyloid processing and integrin signalling pathways. RT-PCR assays were performed to confirm the differential expression of the selected genes.

The methods provided have sufficient specificity and sensitivity to detect metastasis of melanoma. A comparison of the current methods available indicates that tradition methods of H&E and IHC are clinically acceptable whereas, prior to the current invention, PCR methods were unacceptable. Table 1 shows the drawbacks and advantages of current methods prior to the invention claimed herein.

TABLE 1

| Method | Sensitivity | Specificity |
|---|---|---|
| H&E | Low | 100% |
| IHC | Low | 100% |
| PCR | High | Low |

In the present invention, specificity is preferably at least 95%, more preferably, specificity is at least 97% and most preferably, specificity is at least 99% based on a comparison of H&E and IHC negative nodes. Preferably, sensitivity is at least at least 80%, more preferably sensitivity is at least 85% and most preferably, sensitivity is at least 90% based on a comparison of H&E and IHC positive nodes. Preferably, specificity and sensitivity are at least 97% based on a comparison of H&E and IHC negative nodes and at least 85% based on a comparison of H&E and IHC positive nodes, respectively.

Preferably, the pre-determined cut-off levels are at least two-fold over-expression in tissue having metastatic melanoma relative to benign melanocyte or normal tissue.

The preferred methods of the invention employ a rapid technique for extracting nucleic acids from a tissue sample and a method of amplifying and detecting nucleic acid fragments indicative of metastasis. The nucleic acid fragments qualitatively and quantitatively measure mRNA encoded by the Marker genes. Tissue samples include lymph node, both regional and sentinel, skin lesions and other biopsy material.

The methods provided herein allow for intra-operative detection of micrometastases allowing a physician to determine whether to excise additional lymph nodes and to immediately implement an appropriate treatment protocol. As shown in Table 2, if a LN is found to be positive for melanoma, regional LNs are excised and interferon therapy could be suggested. Standard biopsy methods can take over one week and a positive result requires additional surgery to remove LNs and there is a concomitant delay in interferon therapy.

TABLE 2

| Clinical Stage | 1° Tumor (T) | LN | Metastasis | Treatment |
|---|---|---|---|---|
| Stage I | T1: ≦1 mm | Negative | Absent | Excision 1 cm |
| | T2: 1.01-2.00 mm | Negative | Absent | Excision 1-2 cm |
| Stage II | T3: 2.01-4.00 mm | Negative | Absent | Excision 2 cm |
| | T4: >4.01 mm | Negative | Absent | Excision 2 cm |
| Stage III | Any thickness | Positive | Absent | Excision + complete LN |
| | | | | dissection + interferon |
| | | | | clinical trial |
| Stage IV | Any thickness | Positive | Present | Interferon clinical trial, |
| | | | | symptomatic therapy |

It is important to adequately sample the tissue used to conduct the assay. This includes proper excision and processing of the tissue sample as well as extraction of RNA. Once obtained, it is important to process the tissue samples properly so that any cancerous cells present are detected.

In the most preferred embodiment of the invention, node sampling is also given attention both intra- and extra-operatively. Since the distribution of cancer cells in nodes is non-uniform, it is preferable that multiple sections of the node be sampled. Every identified SLN should be submitted for pathological evaluation. SLN material is ordinarily be fixed in formalin and examined as formalin fixed, paraffin embedded tissue sample. Equally representative parts of SLN are processed for molecular analysis (fresh tissue) and histology (fixed tissue). General LN sampling procedures are described in Cochran et al. (2001); and Cochran et al. (2004). One method for accomplishing both a molecular based test and an examination of the same node sample by pathology is to bisect the node through the longest diameter. Each half is then divided into at least four full-faced sections with at least one outer and inner section for pathology as fixed material, and at least one outer and inner section for molecular testing. As the distribution of metastases and micrometastases is not uniform in nodes or other tissues, a sufficiently large sample should be obtained so that metastases will not be missed. One approach to this sampling issue in the present method is to homogenize a large tissue sample, and subsequently perform a dilution of the well-mixed homogenized sample to be used in subsequent molecular testing.

In the case of LN tissue samples, it is preferable to remove any adipose tissue prior to cellular disruption. Manual cell and tissue disruption can be by any means known in the art such as a disposable tissue grinder described in U.S. Pat. No. 4,715,545 or a commercial homogenizer such as Omni GLHl 15 with disposable probes (Omni International, Warrenton, Va.). Homogenization time is within 1 to 2 minutes and is more preferably 30-45 sec. The sample can then be processed to purify the RNA prior to assaying and measuring Marker expression levels. Suitable RNA purification methods include columns such as (e.g., RNeasy mini column, QIAshredder, QIAGEN Inc., Valencia, Calif., or a suitable substitute).

A variety of techniques are available for extracting nucleic acids from tissue samples. Typical commercially available nucleic acid extraction kits take at least 15 minutes to extract the nucleic acid. In the preferred intra-operative methods of the instant invention, nucleic acid is extracted in less than 8 minutes and preferably less than 6 minutes.

The successful isolation of intact RNA generally involves four steps: effective disruption of cells or tissue, denaturation of nucleoprotein complexes, inactivation of endogenous ribonuclease (RNase) and removal of contaminating DNA and protein. The disruptive and protective properties of guanidinium isothiocyanate (GITC) and β-mercaptoethanol (β-me) to inactivate the ribonucleases present in cell extracts make them preferred reagents for the first step. When used in conjunction with a surfactant such as sodium dodecylsulfate (SDS), disruption of nucleoprotein complexes is achieved allowing the RNA to be released into solution and isolated free of protein. Tissues are homogenized in the GITC-containing lysis buffer, addition of ethanol creates the appropriate conditions for RNA to bind to the silica membrane. Centrifugation can clear the lysate of precipitated proteins and cellular DNA and is preferably performed through a column. RNA purification is preferably conducted on a spin column containing silica or other material.

RNA is precipitated via the spin column as described above and centrifugation times are preferably no greater than 30 sec. Typically, the sample is diluted with an equal volume of 70% ethanol and thoroughly mixed prior to applying to the column. After washing, the column is dried by centrifugation, and RNA is eluted in RNase free water and collected by centrifugation. The total time of this rapid protocol is less than 8 minutes and preferably less than 6 min.

In summary the rapid RNA extraction method involves the following steps:
obtaining a tissue sample;
homogenizing the tissue to produce a homogenate;
contacting the homogenate with a substrate containing, or to which is affixed, an RNA-binding material;
allowing the RNA to bind to the RNA binding material;
washing the substrate under conditions sufficient to remove any contaminants, interferents and un-bound RNA; and
eluting bound RNA from the substrate.

The reagents involved in this rapid extraction process can be those provided by the manufacturer or can be, for instance:
Lysis/Binding buffer (preferably, 4.5M GITC, 10 OmM NaPO₄),
Wash buffer I (preferably, 37% ethanol in 5M GITC, 2 OmM Tris-HCl),
Wash buffer II (preferably, 80% ethanol in 2 OmM NaCl, 2 mM Tris-HCl), and
Nuclease-free sterile double distilled water for elution.

In one method, prior to the process for isolating nucleic acids described above, tissue samples are weighed and put into 8 or 14 ml polypropylene culture tubes and pre-cooled on dry ice. The frozen tissue samples are then divided into pieces of about 50 mg or less without being thawed. All buffers are those provided by QIAGEN in the RNeasy mini kit. A volume of homogenization (lysis) buffer is added to the tissue based on Table 3.

TABLE 3

| Tissue Weight (mg) | Homogenization buffer (ml) |
|---|---|
| ≦100 | 2 |
| 100-149 | 2 |
| 150-199 | 3 |
| 200-249 | 4 |
| 250-299 | 5 |
| 300-349 | 6 |
| 350-399 | 7 |
| 400-449 | 8 |
| 450-400 | 9 |
| 500-550 | 10 |
| >550 | * |

* Tissue above 550 mg is divided into equivalent parts and processed as individual samples. An alternative method to calculate lysis buffer volume for tissues over 100 mg is to add 1 ml per 50 mg tissue; using 2 ml for tissues less than 100 mg.

The tissue sample is then homogenized for instance by the Omni GLHl 15 at a power setting to grade 6, Adaptor AlOOO and disposable probes. The homogenate is then mixed with an equal volume of 70% ethanol and thoroughly mixed for instance by vortexing on a VWR Model G560 set at 10 speed (maximum) about 10 seconds or by pipetting 4-5 times. The homogenate/ethanol mixture is then applied to an RNeasy mini column mounted on a vacuum manifold in a volume in accordance with Table 4 so that a consistent amount of the original tissue (approximately 5 mg/column) is loaded thus producing comparable RNA yields for each tissue sample.

TABLE 4

| Tissue weight | Volume homogenate/ethanol |
|---|---|
| (mg) | mix (μl) (recommended) |
| 30-39 | 700 |
| 40-49 | 500 |
| 50-59 | 400 |
| 60-69 | 350 |
| 70-79 | 300 |
| 80-89 | 250 |
| 90-99 | 225 |
| >100 | 200 |

A vacuum is then applied to the column to remove the liquid. The vacuum is stopped and two washes of 700 ml are applied, first with RWI buffer and second with RPE buffer each removed by filtration. Vacuum is at 800-1200 mBar in each case. The column is then placed into a 1.5 ml collection tube and centrifuged in an Eppendorf 5415D centrifuge at 13,200 rpm for 30 seconds to dry. The column is transferred to a new 1.5 ml collection tube. Fifty μl RNase-free water is directly added to the membrane and the column is centrifuged in an Eppendorf 541 5D centrifuge for 30 seconds at 13,200 rpm to elute the RNA. The RNA quality is determined with an Agilent Bioanalyzer and the RNA is stored at −70° C.

Melanin can negatively impact the efficiency of reversed transcription and amplification reactions. Accordingly, a melanin removal process is undertaken when the sample is suspected of containing a significant amount of melanin (as in the case of samples of a primary melanoma or benign skin nevi) and is less of a concern when performing the assay on a SLN since melanocyte content is low. If necessary, melanin is removed to enhance reverse transcription and/or nucleic acid amplification.

Typically, melanin is removed during the filtration steps provided above. In the case of tissue with high melanin concentration, less tissue should be used, approximately 5 mg per Qiagen RNeasy mini column.

If another method is used that results in residual melanin in the sample, removal involves the use of a matrix employing a polymer bead system such as Bio-Gel P-60 (Bio-Rad Laboratories, Hercules, Calif.). Such a method is described by Satyamoorthy et al. (2002). Essentially, this method involves preparing a 50% (w/v) mixture of the Bio-Gel material in 10 mM sodium acetate (pH 4.2). About 300 μl of the mixture are placed in a micro-centrifuge tube and centrifuged at lOOOrpm for 1 min. The supernatant is discarded and the beads are placed in a mini-column or similar vessel. Homogenate is then passed through the vessel containing the beads (after first incubating them in the vessel). The supernatant is collected. Further washing of the beads with additional 100 μl aliquots of 10 mM sodium acetate can be used to capture additional volumes of melanin-free sample if necessary for adequate assay volume. The dark melanin will be clearly visible on the beads retained in the vessel. Other silica-based filters can also be used to remove the melanin pigment as described by Wang et al. (2001).

An important aspect of the intra-operative methods of the invention is rapid Marker detection. Provided that such methods can be conducted within a period acceptable for an intra-operative assay (i.e., no more than about 35 minutes), any reliable, sensitive, and specific method can be used.

In the case of measuring mRNA levels to determine gene expression, assays can be by any means known in the art and include methods such as PCR, Rolling Circle Amplification (RCA), Ligase Chain Reaction (LCR), Strand Displacement Amplification (SDA), Nucleic Acid Sequence Based Amplification (NASBA), and others. The rapid molecular diagnostics involved are most preferably quantitative PCR methods, including QRT-PCR. Detection can be by any method known in the art including microarrays, gene chips and fluorescence.

A typical PCR includes multiple amplification steps, or cycles that selectively amplify target nucleic acid species. A typical PCR includes three steps: a denaturing step in which a target nucleic acid is denatured; an annealing step in which a set of PCR primers (forward and backward primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR includes 20 or more cycles of denaturation, annealing and elongation. Often, the annealing and elongation steps can be performed concurrently, in which case the cycle contains only two steps.

In the preferred inventive method, employing RT-PCR, the RT-PCR amplification reaction is conducted in a time suitable for intra-operative diagnosis, the lengths of each of these steps can be in the seconds range, rather than minutes. Specifically, with certain new thermal cyclers being capable of generating a thermal ramp rate of at least about 5 C° per second, RT-PCR amplifications in 30 minutes or less are used. More preferably, amplifications are conducted in less than 25 minutes. With this in mind, the following times provided for each step of the PCR cycle do not include ramp times. The denaturation step may be conducted for times of 10 seconds or less. In fact, some thermal cyclers have settings for “0 seconds” which may be the optimal duration of the denaturation step. That is, it is enough that the thermal cycler reaches the denaturation temperature. The annealing and elongation steps are most preferably less than 10 seconds each, and when conducted at the same temperature, the combination annealing/elongation step may be less than 10 seconds. Some homogeneous probe detection methods, may require a separate step for elongation to maximize rapid assay performance. In order to minimize both the total amplification time and the formation of non-specific side reactions, annealing temperatures are typically above 50° C. More preferably annealing temperatures are above 55° C.

A single combined reaction for RT-PCR, with no experimenter intervention, is desirable for several reasons: (1) decreased risk of experimenter error; (2) decreased risk of target or product contamination; and (3) increased assay speed. The reaction can consist of either one or two polymerases. In the case of two polymerases, one of these enzymes is typically an RNA-based DNA polymerase (reverse transcriptase) and one is a thermostable DNA-based DNA polymerase. To maximize assay performance, it is preferable to employ a form of “hot start” technology for both of these enzymatic functions. U.S. Pat. Nos. 5,411,876 and 5,985,619 provide examples of different “hot start” approaches. Preferred methods include the use of one or more thermoactivation methods that sequester one or more of the components required for efficient DNA polymerization. U.S. Pat. Nos. 5,550,044 and 5,413,924 describe methods for preparing reagents for use in such methods. U.S. Pat. No. 6,403,341 describes a sequestering approach that involves chemical alteration of one of the PCR reagent components. In the most preferred embodiment, both RNA- and DNA-dependent polymerase activities reside in a single enzyme. Other components that are required for efficient amplification include nucleoside triphosphates, divalent salts and buffer components. In some instances, non-specific nucleic acid and enzyme stabilizers may be beneficial.

In the preferred RT-PCR, the amounts of certain reverse transcriptase and the PCR components are atypical in order to take advantage of the faster ramp times of some thermal cyclers. Specifically, the primer concentrations are very high.

Typical gene-specific primer concentrations for reverse transcriptase reactions are less than about 20 nM. To achieve a rapid reverse transcriptase reaction on the order of one to two minutes, the reverse transcriptase primer concentration is raised to greater than 20 nM, preferably at least about 50 nM, and typically about 100 nM. Standard PCR primer concentrations range from 100 nM to 300 nM. Higher concentrations may be used in standard PCR to compensate for Tm variations. However, for the purposes herein, the referenced primer concentrations are for circumstances where no Tm compensation is needed. Proportionately higher concentrations of primers may be empirically determined and used if Tm compensation is necessary or desired. To achieve rapid PCR, the PCR primer concentrations typically are greater than 250 nM, preferably greater than about 300 nM and typically about 500 nM.

Commercially used diagnostics also preferably employ one or more internal positive control that confirms the operation of a particular amplification reaction in case of a negative result. Potential causes of false negative results that must be controlled in an RT-PCR include: inadequate RNA quantity, degradation of RNA, inhibition of RT and/or PCR and experimenter error.

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

The invention provides specificity and sensitivity sufficient to identify a malignant melanocyte in a tissue sample. The methods determine expression of particularly Marker genes by measuring mRNA encoded by the Markers. The preferred Markers of the invention display at least a two-fold over-expression in tissue having malignant melanocytes relative to benign melanocyte or normal tissue. The results presented herein show that a primary Marker is insufficient to provide clinically relevant information but, when combined with one or more secondary Markers, the information obtained compares to the “gold standard” of H&E and IHC upon which clinicians currently rely. Tertiary Markers and control genes can augment the primary and secondary Markers to further increase specificity and/or sensitivity.

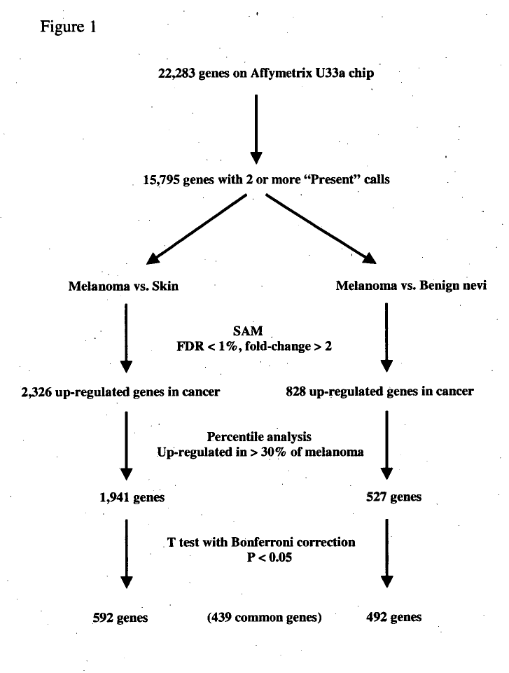

As described in the following Examples, the Markers were identified by the protocol depicted in FIG. 1. Thus, the invention provides a method for identifying melanoma-specific Markers by following the protocol in FIG. 1 and the Examples provided herein.

The primary Marker can be PLAB and is defined herein as the gene encoding any variant, allele etc. including SEQ ID NO: 1. PLAB is also described by Paralkar et al. (1998) and represented by Accession No. AF003934. PLAB is linked to the pathogenesis of prostate cancer (Liu et al (2003); Karan et al. (2003); and Nakamura et al. (2003); U.S. Pat. Nos. 5,994,102; 6,107,476; 6,465,181; 6,500,638; 6,521,227; US Patent Publication Nos. 2002/0048784; 2003/0013097; and 2003/0059431) and colorectal cancer (Brown et al. (2003); Buckhaults et al. (2001); and US Patent Publication No. 2002/0160382).

The secondary Marker is LlCAM and is defined herein as the gene encoding any variant, allele etc. including SEQ ID NO: 2. LlCAM is also described by Haspel et al (2003); and U.S. Pat. Nos. 5,872,225; and 5,969,124 and is represented by Accession No. NM_{—}000425.

The invention further provides tertiary markers that fall into several functional categories. Thus, additional Markers can be used that are found in these functional categories. As described in more detail in the Examples, melanoma-specific up-regulated genes fall into the functional categories of neural tissue development and cell cycle control and melanoma-specific down-regulated genes fall into the functional categories of tissue development and cell differentiation.

The tertiary Markers include SEQ ID NOs: 3, 29-978 and 999. A number of tertiary markers are described in Table 5 and all are summarized in Table 15.

NTRK3 is described by Strausberg et al. (2002); Marchetti et al. (2003); Hisaoka et al. (2002); McGregor et al. (1999); Ryden et al. (1996); U.S. Pat. Nos. 5,348,856; 5,844,092; 5,910,574; and US Patent Publication Nos. 2002/0155480; and 2003/014283 and is represented by Accession No. BC013693 or S76476.1. NTRK3 is also defined as the gene encoding mRNA recognized by the primer/probe sets SEQ ID NOs: 16-18.

Tyrosinase is described by Mandelcorn-Monson et al. (2003); and U.S. Pat. No. 6,153,388 and is represented by Accession No. NM_{—}000372. Tyrosinase is also defined as the gene encoding mRNA recognized by the primer/probe sets SEQ ID NOs: 19-21.

TABLE 5

| Gene | Reference | Accession # |
|---|---|---|
| PBGD | Raich et al. (1986) | NM_000190 |
| CITED1 | Fenner et al. (1998) | NM_004143 |
| PEX6 | Raas-Rothschild et al. (2002) | NM_000287 |
| CAPG | Van Impe et al. (2003) | NM_001747 |
| DUSP4 | Smith et al. (1997) | NM_001394 |
| GDF1 | Ducy et al. (2000) | NM_001492 |
| E2-EpF | Liu et al. (1992) | NM_014501 |
| me20m | Maresh et al. (1994) | U01874 |
| CDH3 | Patel et al. (2003) | NM_001793 |
| SMARCD3 | Ring et al. (1998) | NM_003078 |
| PKM2 | Luftner et al. (2003) | NM_002654 |
| GPI | Tsutsumi et al. (2003) | NM_000175 |
| Pig10 | Polyak et al. (1997) | AF010413 |
| CPEB1 | Welk et al. (2001) | NM_030594 |
| HOXHB9 | Catala et al. (2002) | AI738662 |
| Truncated calcium | The Washington University-Merck | N30649 |
| binding protein | EST Project Hillier et al. (1995) | |
| SAAS | Kikuchi et al. (2003) | NM_013271 |
| HS1-2 | Edgar et al. (2002) | NM_007011 |
| HCN2 | Stieber et al. (2003) | NM_001194 |
| MBP | Kamholz et al. (1996) | M13577 |
| AD3LPAD5 | Li et al. (1995) | U34349 |
| PLOD3 | Wang et al. (2002) | NM_001084 |
| MC1R | Salazar-Onfray et al. (2002) | |
| MIF | Shimizu et al. (1999) | NM_002415 |
| HOXB7 | Care et al. (1996) | NM_004502 |
| AIM1 | Ray et al. (1997) | XM_166300 |
| EpHB6 | Hafner et al. (2003) | NM_004445 |
| AKT1 | Majumder et al. (2004) | NM_005163 |
| AKT2 | Gosmanov et al. (2004) | NM_001626 |
| AKT3 | Xu et al. (2003) | NM_005465 |
| APH-1A | Xu et al. (2003) | NM_016022 |
| APP | Masters et al. (1985) | NM_201414 |
| BACE | Pastorino et al. (2004) | NM_138973 |
| BACE2 | Pastorino et al. (2004) | NM_012104 |
| CAPN1 | Altznauer et al. (2004) | NM_005186 |
| CAPN2 | Alexa et al. (2004) | NM_001748 |
| CDK5 | Qi et al. (2004) | NM_004935 |
| CDK5R1 | Kam et al. (2004) | NM_003885 |
| CSNK1A1 | Burzio et al. (2002) | NM_001892 |
| CSNK1D | Li et al. (2004) | NM_139062 |
| CSNK1E | Swiatek et al. (2004) | NM_152221 |
| CSNK2A1 | Hilgard et al. (2004) | NM_001895 |
| CSNK2A2 | Szebeni et al. (2003) | NM_001896 |
| CSN2K2B | Lee et al. (2004) | NM_001320 |
| GSK3B | Chen et al. (2003) | NM_182946 |
| MAPK1 | Nishihara et al. (2004) | NM_138957 |
| MAPK14 | Bendotti et al. (2004) | NM_139014 |
| MAPK3 | Nishihara et al. (2004) | NM_002746 |
| MAPT | Yu et al. (2004) | NM_016841 |
| NCSTN | Shirotani et al. (2004) | NM_015331 |
| PEN2 | Marlow et al. (2003) | NM_172341 |
| PRKACA | Sakwe et al. (2004) | NM_207518 |
| PRKACB | Dwivedi et al. (2004) | NM_002731 |
| PRKACG | Zhang et al. (2004) | NM_002732 |
| PRKAR1A | Gronholm et al. (2003) | NM_212472 |
| PRKAR2A | MacDougall et al. (2003) | NM_004157 |
| PRKAR2B | Dwivedi et al. (2004) | NM_002736 |
| PRKCE | Schechtman et al. (2004) | NM_005400 |
| PSEN1 | Pitsi et al. (2004) | NM_000021 |
| PSEN2 | Zatti et al. (2004) | NM_012486 |
| PSFL | Clark et al. (2003) | NM_031301 |
| ABL1 | Gustafson et al. (2004) | NM_007313 |
| ACK1 | Ahmed et al. (2004) | NM_005781 |
| ACTN4 | Menez et al. (2004) | NM_004924 |
| ARF1 | Kadaja et al. (2004) | NM_001658 |
| ARPC1B | Kaneda et al. (2002) | |
| BCAR3 | Clark et al. (2003) | NM_003567 |
| BRAF | Sasaki et al. (2004) | NM_004333 |
| CDC42 | Chen et al. (2004) | NM_044472 |
| CRK | Stoletov et al. (2004) | NM_016823 |
| CRKL | Zhang et al. (2003) | NM_005207 |
| DDEF1 | Oda et al. (2003) | NM_018482 |
| DOCK1 | Grimsley et al. (2004) | NM_001380 |
| FYN | Lee et al. (2004) | NM_153048 |
| GIT1 | Haendeler et al. (2003) | NM_014030 |
| GRB2 | Zhou et al. (2004) | NM_203506 |
| GRF2 | Arozarena et al. (2004) | NM_006909 |
| HRAS | Nomura et al. (2004) | NM_005343 |
| JUN | Schmuth et al. (2004) | NM_002228 |
| KRAS2 | Qi et al. (2004) | NM_033360 |
| MAP2K1 | Rhee et al. (2004) | NM_002755 |
| MAP2K2 | Chen et al. (2004) | NM_030662 |
| MAP2K4 | Woo et al. (2004) | NM_003010 |
| MAP3K11 | Zhang et al. (2004a) | NM_002419 |
| MAPK8 | Fujii et al. (2004) | NM_139049 |
| MYLK | Oury et al. (2004) | NM_053032 |
| NRAS | Reifenberger et al. (2004) | NM_002524 |
| PAK1 | Sells et al. (1997) | HSU24152 |
| PAK2 | Kirchhoff et al. (2004) | NM_002577 |
| PAK3 | Kitano et al. (2003) | NM_002578 |
| PAK4 | Barac et al. (2004) | NM_005884 |
| PAK6 | Ching et al. (2003) | NM_020168 |
| PAK7 | Jaffer et al. (2002) | NM_020341 |
| PTK2 | Golubovskaya et al. (2004) | NM_005607 |
| PXN | Saito et al. (2004) | NM_002859 |
| RAC1 | Pontow et al. (2004) | NM_198829 |
| RAF1 | Akula et al. (2004) | NM_002880 |
| RAP1A | Nomura et al. (2004) | NM_002884 |
| RAP2B | Evellin et al. (2002) | NM_002886 |
| SHC1 | Yannoni et al. (2004) | NM_183001 |
| SOS1 | Buchs et al. (2004) | NM_005633 |
| SRC | Encinas et al. (2004) | NM_198291 |
| TLN1 | Tremuth et al. (2004) | NM_006289 |
| VASP | Tokuo et al. (2004) | NM_003370 |
| VCL | Izard et al. (2004) | NM_003373 |
| WASPIP | Luthi et al. (2003) | NM_003387 |
| ZYX | Li et al. (2004) | NM_003461 |

Tertiary Markers are able to replace and/or supplement primary or secondary Markers provided that the resulting assays have adequate sensitivity and specificity.

The specificity of any given amplification-based molecular diagnostic relies heavily, but not exclusively, on the identity of the primer sets. The primer sets are pairs of forward and reverse oligonucleotide primers that anneal to a target DNA sequence to permit amplification of the target sequence, thereby producing a target sequence-specific amplicon. The primers must be capable of amplifying Markers of the disease state of interest. In the case of the instant invention, these Markers are directed to melanoma.

The reaction must also contain some means of detection of a specific signal. This is preferably accomplished through the use of a reagent that detects a region of DNA sequence derived from polymerization of the target sequence of interest. Preferred reagents for detection give a measurable signal differential when bound to a specific nucleic acid sequence of interest. Often, these methods involve nucleic acid probes that give increased fluorescence when bound to the sequence of interest. Typically, the progress of the reactions of the inventive methods are monitored by analyzing the relative rates of amplicon production for each PCR primer set.

The invention further includes primer/probe sets and their use in the claimed methods. The sequences are:

SEQ ID NO:4 (PLAB forward primer)
ggcagaatcttcgtccgca
SEQ ID NO:5 (PLAB reverse primer)
ggacagtggtccccgttg
SEQ ID NO.6 (PLAB probe)
cccagctggagttgcacttgcggcc
SEQ ID NO:7 (PLAB upper primer)
gaacaccgacctcgtccc
SEQ ID NO:8 (PLAB lower primer)
ggcggcccgagagata
SEQ ID NO:9 (PLAB probe)
cgccagaagtgcggctgggattt
SEQ ID NO:10 (L1CAM forward)
gctgggactgggaacagaact
SEQ ID NO:11 (L1CAM Reverse)
ggagcagagatggcaaagaaa
SEQ ID NO:12 (L1CAM probe)
ttccccaccatctgctgt
SEQ ID NO:13 (L1CAM upper)
ccacagatgacatcagcctcaa
SEQ ID NO:14 (L1CAM lower)
ggtcacacccagctcttcctt
SEQ ID NO:15 (L1CAM probe)
tggcaagcccgaagtgcagttcctt
SEQ ID NO:16 (NTRK3 primer)
gccccggcacccttta
SEQ ID NO:17 (NTRK3 primer)
aaccctgccagtggtggat
SEQ ID NO:18 (NTRK3 probe)
cagatgggtgttttc
SEQ ID NO:19 (Tyr upper)
actcagcccagcatcattcttc
SEQ ID NO:20 (Tyr lower)
atggctgttgtactcctccaatc
SEQ ID NO:21 (Tyr probe)
cttctcctcttggcagattgtctgtagctt
SEQ ID NO:22 (PBGD upper)
ccacacacagcctactttccaa
SEQ ID NO:23 (PBGD lower)
tacccacgcgaatcactctca
SEQ ID NO:24 (PBGD probe)
aacggcaatgcggctgcaacggcggaatt

Monitoring amplicon production may be achieved by a number of detection reagents and methods, including without limitation, fluorescent primers, and fluorogenic probes and fluorescent dyes that bind double-stranded DNA. Molecular beacons, Scorpions, and other detection schemes may also be used. A common method of monitoring a PCR employs a fluorescent hydrolysis probe assay. This method exploits the 5′ nuclease activity of certain thermostable DNA polymerases (such as Taq or TfI DNA polymerases) to cleave an oligomeric probe during the PCR process.

The invention further provides amplicons obtained by PCR methods utilized in the inventive methods. These amplicons include the following:

SEQ ID NO:25 (PLAB Amplicon)
gaacaccgacctcgtcccggcccctgcagtccggatactcacgccagaag
tgcggctgggatccggcggccacctgcacctgcgtatctctcgggccgcc
SEQ ID NO:26 (L1CAM Amplicon)
ccacagatgacatcagcctcaagtgtgaggccagtggcaagcccgaagtg
cagttccgctggacgagggatggtgtccacttcaaacccaaggaagagct
gggtgtgacc
SEQ ID NO:27 (tyrosinase Amplicon)
actcagcccagcatcattcttctcctcttggcagattgtctgtagccgat
tggaggagtacaacagccat
SEQ ID NO:28 (PBGD Amplicon)
ccacacacagcctactttccaagcggagccatgtctggtaacggcaatgc
ggctgcaacggcggaagaaaacagcccaaagatgagagtgattcgcgtgg
gta

The oligomer is selected to anneal to the amplified target sequence under elongation conditions. The probe typically has a fluorescent reporter on its 5′ end and a fluorescent quencher of the reporter at the 3′ end. So long as the oligomer is intact, the fluorescent signal from the reporter is quenched. However, when the oligomer is digested during the elongation process, the fluorescent reporter is no longer in proximity to the quencher. The relative accumulation of free fluorescent reporter for a given amplicon may be compared to the accumulation of the same amplicons for a control sample and/or to that of a control gene, such as, without limitation, β-Actin or PBGD to determine the relative abundance of a given cDNA product of a given RNA in a RNA population. Products and reagents for the fluorescent hydrolysis probe assay are readily available commercially, for instance from Applied Biosystems.

Suitable detection reagents are commonly referred to as “Scorpions” and are described in U.S. Pat. Nos. 6,326,145 and 5,525,494. These reagents include one or more molecules comprising a tailed primer and an integrated signaling system. The primer has a template binding region and a tail comprising a linker and a target binding region. The target binding region in the tail hybridizes to complementary sequence in an extension product of the primer. This target specific hybridization event is coupled to a signaling system wherein hybridization leads to a detectable change. In PCR the target binding region and the tail region are advantageously arranged such that the tail region remains single stranded, i.e. uncopied. Thus the tail region is non-amplifiable in the PCR amplification products. The linker comprises a blocking moiety that prevents polymerase mediated chain extension on the primer template.

The most preferred detection reagents are TaqMan® probes (Roche Diagnostics, Branchburg, N.J.) and they are described in U.S. Pat. Nos. 5,210,015; 5,487,972; and 5,804,375. Essentially, these probes involve nucleic acid detection by virtue of the separation of a fluor-quencher combination on a probe through the 5′-3′ exonuclease activity of the polymerase used in the PCR. Any suitable fluorophore can be used for any of the Markers or controls. Such fluorophores include, without limitation, Texas Red, CaI Red, Fam, Cy3 and Cy5. In one embodiment, the following fluorophores correspond to the noted Markers: PLAB: Fam; LlCAM: Texas Red or CaI Red, tyrosinase: Cl; PBGD: Cy5.

Equipment and software also are readily available for controlling and monitoring amplicon accumulation in PCR and QRT-PCR including the Smart Cycler thermocylcer commercially available from Cepheid of Sunnyvale, Calif., and the ABI Prism 7700 Sequence Detection System, commercially available from Applied Biosystems.

In the case of gene expression assays, it is preferable to use a gene constitutively expressed in the tissue of interest. PBGD is commonly used as an internal control due to several factors: it contains no known pseudogenes in humans, it is constitutively expressed in human tissues and it is expressed at a relatively low level and therefore is less likely to cause inhibition of the amplification of target sequences of interest. Use of PBGD as a control minimizes or eliminates reporting erroneous results arising from all potential sources of false negative results.

In the commercialization of the described methods for QRT-PCR certain kits for detection of specific nucleic acids are particularly useful. In one embodiment, the kit includes reagents for amplifying and detecting Markers. Optionally, the kit includes sample preparation reagents and or articles (e.g., tubes) to extract nucleic acids from lymph node tissue. The kits may also include articles to minimize the risk of sample contamination (e.g., disposable scalpel and surface for lymph node dissection and preparation).

In a preferred kit, reagents necessary for the one-tube QRT-PCR process described above are included such as reverse transcriptase, a reverse transcriptase primer, a corresponding PCR primer set (preferably for Markers and controls), a thermostable DNA polymerase, such as Taq polymerase, and a suitable detection reagent(s), such as, without limitation, a scorpion probe, a probe for a fluorescent hydrolysis probe assay, a molecular beacon probe, a single dye primer or a fluorescent dye specific to double-stranded DNA, such as ethidium bromide. The primers are preferably in quantities that yield the high concentrations described above. Thermostable DNA polymerases are commonly and commercially available from a variety of manufacturers. Additional materials in the kit may include: suitable reaction tubes or vials, a barrier composition, typically a wax bead, optionally including magnesium; reaction mixtures (typically 1 OX) for the reverse transcriptase and the PCR stages, including necessary buffers and reagents such as dNTPs; nuclease-or RNase-free water; RNase inhibitor; control nucleic acid(s) and/or any additional buffers, compounds, co-factors, ionic constituents, proteins and enzymes, polymers, and the like that may be used in reverse transcriptase and/or PCR stages of QRT-PCR. Optionally, the kits include nucleic acid extraction reagents and materials. Instructions are also preferably included in the kits.

The following examples are provided to illustrate but not limit the claimed invention. AU references cited herein are hereby incorporated herein by reference.

### EXAMPLE 1

### Tissue Preparation

Fresh frozen malignant melanoma, benign skin nevi, normal skin, melanoma lymph node metastasis and melanoma-free lymph node samples were obtained from Genomics Collaborative, Inc. (Cambridge, Mass.), Asterand (Detroit, Mich.), Clinomics (Pittsfield, Mass.) and Proteogenex (Los Angeles, Calif.), Ardais (Lexington, Mass.) and Impath (Westborough, Mass.). All tissue vendors declared that tissue specimens used in the study were collected according to an Institutional Review Board approved protocol of corresponding hospitals and principles of bioethics. Patients' demographic and pathology information was also collected. The histopathological features of each sample were reviewed to confirm diagnosis, and to estimate sample preservation and tumor content.

Melanoma and benign nevi primary tissues chosen for microarray analysis had melanocyte content greater than 50% with no mixed histology. Melanoma positive lymph nodes were collected from malignant melanoma patients; diagnosis of melanoma was confirmed by H&E in combination with IHC (SlOO and HMB45). Melanoma free lymph nodes derived from patients that did not have melanoma in their clinical history and absence of melanoma was confirmed by H&E and IHC using antibodies for SlOO and HMB45.

RNA from a total of 70 primary tissue samples was used for gene expression profiling and melanoma specific gene identification. Samples included 45 primary malignant melanoma, 18 benign skin nevi, and 7 normal skin tissues. The majority of primary melanomas included in the study represent early stage of disease and have thickness less than 4 mm, which is consistent with the standard melanoma patient population. Aitken et al. (2004). Patient demographic, clinical and pathology characteristics are presented in Table 6 and summarized in Table 7.

In addition, 77 malignant melanoma LN metastasis and 18 melanoma-free LN tissue samples were used for one-step quantitative PCR assay. Melanoma positive lymph nodes included axillary, cervical and inguinal lymph nodes with metastasis derived from epithelioid and spindle cell primary melanomas. Out of 18 melanoma free LN, 10 were collected from other cancer patients but no cancer cells were found in these nodes by pathologists and 8 LN were from non-malignant lesions.

TABLE 6

| Sample | | | | | | T | N&M | Clark |
|---|---|---|---|---|---|---|---|---|
| ID | Age | Gender | Race | Diagnosis | Location | Stage | Stage | level |
| 430MM | n/a | F | Cau | normal skin | trunk | | | |
| 431MM | n/a | F | Cau | normal skin | trunk | | | |
| 432MM | n/a | F | Cau | normal skin | trunk | | | |
| 433MM | n/a | F | Cau | normal skin | trunk | | | |
| 435MM | n/a | F | Cau | normal skin | trunk | | | |
| 437MM | n/a | F | Cau | normal skin | trunk | | | |
| 485MM | 37 | M | Cau | normal skin | skin, NOS | | | |
| 487MM | 35 | F | Cau | atypical nevus, mild | face | | | |
| 489MM | 56 | F | Cau | compound nevus | face | | | |
| 490MM | 16 | F | Cau | compound nevus | scalp & neck | | | |
| 491MM | 15 | M | Cau | compound nevus | trunk | | | |
| 493MM | 35 | F | Cau | compound nevus | trunk | | | |
| 495MM | 18 | F | Cau | benign nevus, NOS | trunk | | | |
| 496MM | 21 | F | Cau | intradermal nevus | lower limb & hip | | | |
| 497MM | 12 | M | Cau | intradermal nevus | lower limb & hip | | | |
| 498MM | 44 | F | Cau | benign nevus, NOS | trunk | | | |
| 499MM | 48 | F | Cau | benign nevus, NOS | face | | | |
| 500MM | 65 | M | Cau | intradermal nevus | trunk | | | |
| 501MM | 30 | M | Cau | compound nevus | lower limb & hip | | | |
| 502MM | 20 | F | Cau | compound nevus | trunk | | | |
| 503MM | 35 | M | Cau | intradermal nevus | lower limb & hip | | | |
| 504MM | 23 | M | Cau | compound nevus | trunk | | | |
| 507MM | 53 | M | Cau | atypical nevus, moderate | trunk | | | |
| 508MM | 28 | M | Cau | compound nevus | trunk | | | |
| 509MM | 43 | M | Cau | intradermal nevus | trunk | | | |
| 392MM | 58 | F | Cau | epithelioid melanoma | trunk | T3 | N0M0 | 4 |
| 397MM | 51 | F | Cau | epithelioid melanoma | lower limb & hip | T2 | N0M0 | 3 |
| 405MM | 46 | M | Cau | epithelioid melanoma | upper limb & | T2 | N0M0 | 3 |
| | | | | | shoulder | | | |
| 407MM | 64 | F | Cau | epithelioid melanoma | trunk | T1 | N0M0 | 2 |
| 409MM | 54 | F | Cau | epithelioid melanoma | scalp & neck | T2 | N0M0 | 3 |
| 440MM | 61 | M | Cau | malignant melanoma, | lower limb & hip | T1 | N0M0 | 2 |
| | | | | NOS | | | | |
| 441MM | 78 | M | Cau | spindle cell melanoma | face | T4 | N0M0 | 5 |
| 442MM | 52 | M | Cau | malignant melanoma, | upper limb & | T2 | N0M0 | 3 |
| | | | | NOS | shoulder | | | |
| 443MM | 51 | F | Cau | spindle cell melanoma | trunk | T2 | N0M0 | 3 |
| 444MM | 49 | F | Cau | spindle cell melanoma | lower limb & hip | T3 | N0M0 | 4 |
| 445MM | 76 | F | Cau | malignant melanoma, | upper limb & | T3 | N0M0 | 4 |
| | | | | NOS | shoulder | | | |
| 446MM | 86 | M | Cau | malignant melanoma, | scalp & neck | T1 | N0M0 | 2 |
| | | | | NOS | | | | |
| 447MM | 48 | M | Cau | epithelioid melanoma | skin, NOS | T3 | N0M0 | 4 |
| 448MM | 72 | F | Cau | epithelioid melanoma | upper limb & | T2 | N0M0 | 3 |
| | | | | | shoulder | | | |
| 449MM | 62 | M | Asian | epithelioid melanoma | lower limb & hip | T3 | N1M0 | n/a |
| 450MM | 90 | F | Cau | epithelioid melanoma | upper limb & | T4 | N1M1 | n/a |
| | | | | | shoulder | | | |
| 452MM | 43 | M | Cau | epithelioid melanoma | skin, NOS | T3 | N0M0 | n/a |
| 453MM | 48 | F | Cau | epithelioid melanoma | trunk | T3 | N0M0 | n/a |
| 454MM | 69 | M | Cau | epithelioid melanoma | upper limb & | T3 | N0M0 | n/a |
| | | | | | shoulder | | | |
| 455MM | 55 | M | Cau | malignant melanoma, | skin, NOS | T2 | N0M0 | n/a |
| | | | | NOS | | | | |
| 456MM | 63 | M | Cau | malignant melanoma, | lower limb & hip | T2 | N0M0 | 3 |
| | | | | NOS | | | | |
| 457MM | 69 | M | Cau | spindle cell melanoma | trunk | T1 | N0M0 | 2 |
| 459MM | 86 | F | Cau | malignant melanoma, | lower limb & hip | T2 | N0M0 | 3 |
| | | | | NOS | | | | |
| 460MM | 64 | M | Cau | malignant melanoma, | upper limb & | T3 | N0M0 | 4 |
| | | | | NOS | shoulder | | | |
| 461MM | 66 | M | Cau | epithelioid melanoma | trunk | T1 | N0M0 | 2 |
| 463MM | 58 | M | Cau | malignant melanoma, | trunk | T1 | N0M0 | 2 |
| | | | | NOS | | | | |
| 464MM | 53 | M | Cau | epithelioid melanoma | face | T2 | N0M0 | 3 |
| 465MM | 77 | F | Cau | epithelioid melanoma | upper limb & | T3 | N0M0 | 4 |
| | | | | | shoulder | | | |
| 466MM | 79 | F | Cau | malignant melanoma, | upper limb & | T1 | N0M0 | 2 |
| | | | | NOS | shoulder | | | |
| 468MM | 86 | F | Cau | spindle cell melanoma | upper limb & | T2 | N0M0 | 3 |
| | | | | | shoulder | | | |
| 469MM | 43 | F | Cau | malignant melanoma, | scalp & neck | T1 | N0M0 | 2 |
| | | | | NOS | | | | |
| 470MM | 81 | M | Cau | malignant melanoma, | upper limb & | T2 | N0M0 | 3 |
| | | | | NOS | shoulder | | | |
| 472MM | 38 | F | Cau | spindle cell melanoma | upper limb & | T1 | N0M0 | 2 |
| | | | | | shoulder | | | |
| 473MM | 69 | F | Cau | malignant melanoma, | upper limb & | T1 | N0M0 | 3 |
| | | | | NOS | shoulder | | | |
| 475MM | 77 | F | Cau | malignant melanoma, | face | T3 | N0M0 | 4 |
| | | | | NOS | | | | |
| 476MM | 87 | F | Cau | spindle cell melanoma | upper limb & | T3 | N0M0 | 4 |
| | | | | | shoulder | | | |
| 477MM | 82 | M | Cau | malignant melanoma, | scalp & neck | T2 | N0M0 | 3 |
| | | | | NOS | | | | |
| 478MM | 78 | F | Cau | epithelioid melanoma | face | T3 | N0M0 | 4 |
| 480MM | 59 | M | Cau | malignant melanoma, | upper limb & | T2 | N0M0 | 3 |
| | | | | NOS | shoulder | | | |
| 481MM | 85 | M | Cau | malignant melanoma, | upper limb & | T3 | N0M0 | 4 |
| | | | | NOS | shoulder | | | |
| 482MM | 66 | M | Cau | epithelioid melanoma | face | T3 | N0M0 | 4 |
| 483MM | 85 | F | Cau | epithelioid melanoma | trunk | T4 | N0M0 | 5 |
| 484MM | 70 | F | Cau | malignant melanoma, | upper limb & | T1 | N0M0 | 3 |
| | | | | NOS | shoulder | | | |
| 511MM | 69 | M | Cau | epithelioid melanoma | skin, NOS | T3 | N1M0 | 4 |
| 512MM | 45 | M | Cau | epithelioid melanoma | trunk | T4 | N0M0 | 3 |

TABLE 7

| | | | Normal |
|---|---|---|---|
| Characteristics | Melanoma (%) | Nevi (%) | skin (%) |
| Mean Age | 65.51 ± 14.55 | 33.17 ± 15.60 | n/a |
| Gender | | | |
| Female | 22 (48.9) | 9 (50) | 6 |
| Male | 23 (51.1) | 0 (50) | 1 |
| Anatomical location | | | |
| Face | 5 (11.1) | 3 (17) | |
| Scalp and neck | 4 (8.( ) | 1 (6) | |
| Trunk | 9 (20) | 10 (55) | 6 (86) |
| Upper limb and shoulder | 17 (37.8) | | |
| Lower limb and hip | 6 (13.3) | 4 (22) | |
| Skin, NOS | 4 (8.9) | | 1 (14) |
| Histological diagnosis | | | |
| Epitheloid cell | 20 (44.4) | | |
| Spindle cell | 7 (15.6) | | |
| Malignant melanoma NOS | 18 (40) | | |
| Compound nevus | | 8 (44) | |
| Intradermal nevus | | 5 (28) | |
| Atypical nevus | | 2 (11) | |
| Benign nevus, NOS | | 3 (17) | |
| Normal skin | | | 7 (100) |
| T stage (thickness) | | | |
| T1 | 11 (24.4) | | |
| T2 | 14 (31.1) | | |
| T3 | 16 (35.6) | | |
| T4 | 4 (8.9) | | |
| N stage | | | |
| N0 | 42 (93.3) | | |
| N1 | 3 (6.7) | | |
| M stage | | | |
| M0 | 44 (97.8) | | |
| M1 | 1 (2.2) | | |
| Clark level | | | |
| 2 | 9 (20) | | |
| 3 | 16 (35/6) | | |
| 4 | 12 (26.7) | | |
| 5 | 2 (4.4) | | |
| n/a | 6 (13.3) | | |

### EXAMPLE 2

### RNA Isolation from Malignant Melanoma and Benign Skin Nevi Samples

Qiagen RNeasy™ Mini Kit (QIAGEN Inc., Valencia, Calif.) was used, with a 5 modified protocol to minimize the residual melanin in the RNA sample. For melanocyte containing tissues, four replicate tissue samples derived from individual patient each weighed approximately 5 mg and were used and processed separately. Tissue samples were homogenized in 1.0 ml RLT buffer (QIAGEN) containing 10 μl β-mercaptoethanol (Sigma Chemical Co., St. Louis, Mo.) by a mechanical homogenizer (UltraTurrex T8, IKA-Werke, Staufen, Germany). After homogenization, samples were loaded onto QIAGEN RNeasy™ columns and followed by centrifugation. After discarding the flow-through, 700 ml of RWl buffer was added; the column was kept for 5 min at room temperature and then centrifuged. This step was repeated 3 times. Then the standard QIAGEN RNeasy™ Mini Kit protocol was followed. To remove RNA from the silica gel membrane, a two-step elution was performed. The total RNA derived from the same individual patient tissue was pooled and used for further analysis.

Standard Trizol protocol was used for RNA isolation from tissues that do not contain a significant proportion of melanocytes. Tissue was homogenized in Trizol reagent (Invitrogen, Carlsbad, Calif.). After centrifugation the top liquid phase was collected and total RNA was precipitated with isopropyl alcohol at −20° C. RNA pellets were washed with 75% ethanol, resolved in water and stored at −80° C. until use. RNA quality was examined with an Agilent 2100 Bioanalyzer RNA 6000 Nano Assay (Agilent Technologies, Palo Alto, Calif.).

Labeled cRNA was prepared and hybridized with the high-density oligonucleotide array HuI 33A Gene Chip (Affymetrix, Santa Clara, Calif.) containing a total of 22,000 probe sets according to the standard manufacturer protocol. Arrays were scanned using Affymetrix protocols and scanners. For subsequent analysis, each probe set was considered as a separate gene. Expression values for each gene were calculated by using Affymetrix Gene Chip analysis software MAS 5.0. All chips met three quality control standards: “present” call was greater than 35%, scale factor was smaller than 12 when scaled to a target intensity of 600, and background level was less than 150. Lower than usual percent of “present” calls cut-off was chosen because it is difficult to isolate RNA from skin cells (Hipfel et al. (1998)) resulting in lower overall gene expression levels.

### EXAMPLE 3

### Data Analysis

Gene expression data were filtered to include only genes called “present” in 2 or more samples. This filter was used to remove genes that did not change expression in the samples. Of the 22,000 genes presented on the array, 15,795 passed this filter and were used for hierarchical clustering. Prior to clustering, each gene expression signal was divided by the median expression in al samples in the data set. This standardization step minimized the effect of the magnitude of gene expression and group together genes with similar expression patterns in the clustering analysis. Average linkage hierarchical clustering using Pearson correlation was performed on both the genes and the samples using GeneSpring 6.1.

In order to identify differentially expressed genes, we compared the melanoma samples to the benign nevi and the normal skin samples separately. The first analysis consisted of the 45 melanoma and 7 normal skin samples; the second analysis consisted of 45 melanoma and 18 nevi samples. These two datasets were analyzed separately in following procedures as shown in FIG. 1. Significance analysis of microarray (SAM; Tusher et al. (2001)) and Student T-test were used in gene selection. Parameters for SAM were set as Δ=2.5 and fold change=2.0 with 1,000 permutations. FDR was 1%. There were no missing data and the default random number was used. Next percentile analysis was conducted. For up-regulated genes the 30% ile in melanoma samples was compared to the maximum of the normal samples, or that of nevi samples. Student T-test with Bonferroni correction was also performed with cut-off p<0.05 in order to ensure that the selected genes had significant differential expression between the two groups of the samples. As a final step, we identified common genes between the melanoma/benign and melanoma/normal gene lists resulting in the single list of genes upregulated in melanoma shown in FIG. 1 where the 439 common genes correspond to SEQ ID NOs: 29-467 as described in Table 15 with the results shown in Table 8.

TABLE 8

| | Median | | |
|---|---|---|---|
| | Expression in | Fold Change | Fold Change |
| PSID | Melanoma | (Cancer vs Benign) | (Cancer vs skin) |
| 200078_s_at | 3954 | 2 | 3 |
| 200601_at | 9254 | 2 | 7 |
| 200612_s_at | 2396 | 2 | 5 |
| 200644_at | 7240 | 3 | 6 |
| 200660_at | 14659 | 3 | 4 |
| 200707_at | 3153 | 2 | 51 |
| 200736_s_at | 7305 | 3 | 3 |
| 200737_at | 2423 | 2 | 2 |
| 200783_s_at | 1028 | 2 | 2 |
| 200825_s_at | 3746 | 3 | 3 |
| 200827_at | 1593 | 2 | 2 |
| 200837_at | 5817 | 2 | 5 |
| 200838_at | 19225 | 8 | 17 |
| 200839_s_at | 28353 | 5 | 7 |
| 200859_x_at | 9665 | 3 | 7 |
| 200910_at | 7780 | 2 | 4 |
| 200950_at | 8419 | 3 | 7 |
| 200954_at | 3132 | 2 | 6 |
| 200966_x_at | 27388 | 2 | 3 |
| 200967_at | 6154 | 2 | 4 |
| 200968_s_at | 5587 | 2 | 7 |
| 200972_at | 8943 | 3 | 3 |
| 201038_s_at | 1480 | 2 | 2 |
| 201051_at | 5439 | 2 | 5 |
| 201105_at | 33285 | 4 | 5 |
| 201106_at | 7546 | 2 | 4 |
| 201188_s_at | 1730 | 2 | 8 |
| 201189_s_at | 3870 | 3 | 3 |
| 201195_s_at | 6005 | 4 | 18 |
| 201202_at | 1860 | 3 | 2 |
| 201251_at | 23965 | 5 | 11 |
| 201252_at | 901 | 2 | 3 |
| 201271_s_at | 1648 | 2 | 5 |
| 201291_s_at | 601 | 3 | 42 |
| 201313_at | 2414 | 5 | 6 |
| 201346_at | 3359 | 2 | 2 |
| 201393_s_at | 2166 | 2 | 5 |
| 201416_at | 4845 | 4 | 4 |
| 201417_at | 2905 | 2 | 4 |
| 201470_at | 18525 | 4 | 4 |
| 201474_s_at | 2471 | 5 | 22 |
| 201485_s_at | 1398 | 2 | 10 |
| 201486_at | 1105 | 2 | 3 |
| 201536_at | 2441 | 2 | 4 |
| 201614_s_at | 651 | 2 | 4 |
| 201660_at | 4713 | 7 | 3 |
| 201661_s_at | 3336 | 6 | 5 |
| 201662_s_at | 2693 | 5 | 3 |
| 201670_s_at | 3047 | 2 | 12 |
| 201714_at | 993 | 2 | 3 |
| 201765_s_at | 5475 | 4 | 5 |
| 201792_at | 3897 | 3 | 4 |
| 201804_x_at | 5609 | 3 | 3 |
| 201819_at | 2575 | 5 | 3 |
| 201850_at | 11103 | 10 | 20 |
| 201880_at | 1833 | 2 | 3 |
| 201910_at | 3854 | 4 | 4 |
| 201911_s_at | 2154 | 3 | 4 |
| 201931_at | 2578 | 3 | 2 |
| 201954_at | 25901 | 9 | 15 |
| 201976_s_at | 7697 | 2 | 6 |
| 202069_s_at | 855 | 2 | 3 |
| 202070_s_at | 2490 | 4 | 5 |
| 202111_at | 1380 | 3 | 9 |
| 202154_x_at | 10260 | 3 | 4 |
| 202185_at | 8493 | 4 | 6 |
| 202188_at | 1269 | 3 | 9 |
| 202219_at | 5630 | 3 | 7 |
| 202224_at | 2650 | 3 | 4 |
| 202225_at | 1534 | 2 | 2 |
| 202260_s_at | 7877 | 6 | 6 |
| 202295_s_at | 15129 | 6 | 4 |
| 202329_at | 3438 | 4 | 4 |
| 202367_at | 898 | 2 | 2 |
| 202370_s_at | 4669 | 3 | 2 |
| 202478_at | 7922 | 3 | 11 |
| 202503_s_at | 2424 | 3 | 5 |
| 202589_at | 3494 | 2 | 6 |
| 202603_at | 3772 | 3 | 2 |
| 202705_at | 1058 | 4 | 4 |
| 202737_s_at | 2865 | 3 | 4 |
| 202779_s_at | 3400 | 9 | 55 |
| 202785_at | 1134 | 2 | 6 |
| 202862_at | 2540 | 6 | 4 |
| 202898_at | 4736 | 4 | 63 |
| 202954_at | 1357 | 2 | 2 |
| 202958_at | 2401 | 4 | 3 |
| 202961_s_at | 19648 | 4 | 5 |
| 202986_at | 2052 | 3 | 28 |
| 203011_at | 1346 | 2 | 2 |
| 203022_at | 1068 | 2 | 2 |
| 203069_at | 560 | 15 | 13 |
| 203071_at | 958 | 19 | 3 |
| 203094_at | 968 | 2 | 2 |
| 203145_at | 513 | 2 | 2 |
| 203167_at | 2523 | 3 | 5 |
| 203217_s_at | 6416 | 3 | 5 |
| 203234_at | 725 | 3 | 3 |
| 203256_at | 7799 | 5 | 31 |
| 203262_s_at | 2482 | 2 | 2 |
| 203300_x_at | 5278 | 3 | 16 |
| 203315_at | 3273 | 2 | 2 |
| 203366_at | 847 | 2 | 2 |
| 203396_at | 3946 | 2 | 3 |
| 203452_at | 579 | 4 | 12 |
| 203456_at | 1132 | 2 | 3 |
| 203502_at | 657 | 3 | 3 |
| 203518_at | 2943 | 3 | 7 |
| 203554_x_at | 5056 | 3 | 4 |
| 203557_s_at | 839 | 2 | 2 |
| 203570_at | 2553 | 4 | 15 |
| 203590_at | 3093 | 3 | 7 |
| 203643_at | 1380 | 3 | 8 |
| 203663_s_at | 9720 | 5 | 3 |
| 203668_at | 1963 | 2 | 3 |
| 203693_s_at | 588 | 3 | 3 |
| 203695_s_at | 1548 | 6 | 3 |
| 203723_at | 4087 | 5 | 9 |
| 203729_at | 9154 | 2 | 14 |
| 203730_s_at | 517 | 3 | 3 |
| 203731_s_at | 691 | 3 | 2 |
| 203775_at | 1251 | 4 | 3 |
| 203827_at | 3380 | 11 | 8 |
| 203878_s_at | 2036 | 8 | 5 |
| 204014_at | 7184 | 10 | 46 |
| 204015_s_at | 2207 | 5 | 15 |
| 204033_at | 2154 | 7 | 7 |
| 204092_s_at | 664 | 3 | 3 |
| 204099_at | 2496 | 6 | 6 |
| 204170_s_at | 1362 | 3 | 4 |
| 204197_s_at | 2889 | 3 | 4 |
| 204198_s_at | 4024 | 3 | 3 |
| 204202_at | 875 | 3 | 3 |
| 204228_at | 1299 | 2 | 3 |
| 204244_s_at | 538 | 4 | 4 |
| 204247_s_at | 689 | 3 | 27 |
| 204252_at | 5294 | 5 | 7 |
| 204262_s_at | 1003 | 4 | 5 |
| 204423_at | 689 | 4 | 3 |
| 204436_at | 3113 | 3 | 3 |
| 204458_at | 908 | 3 | 3 |
| 204467_s_at | 1910 | 3 | 6 |
| 204584_at | 9677 | 21 | 15 |
| 204585_s_at | 805 | 13 | 17 |
| 204647_at | 1693 | 3 | 3 |
| 204654_s_at | 3894 | 4 | 25 |
| 204709_s_at | 312 | 24 | 16 |
| 204778_x_at | 537 | 2 | 4 |
| 204779_s_at | 1641 | 4 | 4 |
| 204857_at | 2736 | 3 | 297 |
| 204932_at | 241 | 3 | 3 |
| 204973_at | 1478 | 3 | 7 |
| 204995_at | 496 | 4 | 6 |
| 205051_s_at | 3875 | 4 | 3 |
| 205142_x_at | 937 | 2 | 3 |
| 205169_at | 267 | 2 | 7 |
| 205373_at | 579 | 10 | 8 |
| 205376_at | 815 | 2 | 3 |
| 205405_at | 2302 | 4 | 11 |
| 205447_s_at | 567 | 13 | 5 |
| 205458_at | 3288 | 6 | 8 |
| 205566_at | 2684 | 7 | 8 |
| 205591_at | 1190 | 5 | 3 |
| 205681_at | 1316 | 8 | 12 |
| 205690_s_at | 9179 | 8 | 9 |
| 205691_at | 226 | 7 | 5 |
| 205717_x_at | 8127 | 3 | 3 |
| 205813_s_at | 430 | 11 | 9 |
| 205937_at | 301 | 7 | 5 |
| 205945_at | 772 | 2 | 4 |
| 205996_s_at | 909 | 2 | 3 |
| 206128_at | 364 | 3 | 3 |
| 206307_s_at | 534 | 8 | 4 |
| 206332_s_at | 4671 | 2 | 2 |
| 206397_x_at | 1436 | 6 | 43 |
| 206441_s_at | 3681 | 6 | 31 |
| 206462_s_at | 9953 | 53 | 24 |
| 206503_x_at | 419 | 8 | 9 |
| 206617_s_at | 898 | 4 | 19 |
| 206630_at | 23194 | 3 | 46 |
| 206688_s_at | 2989 | 3 | 2 |
| 206696_at | 6446 | 7 | 191 |
| 206777_s_at | 683 | 4 | 7 |
| 206864_s_at | 421 | 5 | 5 |
| 206976_s_at | 3375 | 4 | 3 |
| 207038_at | 1986 | 9 | 47 |
| 207060_at | 497 | 4 | 5 |
| 207144_s_at | 593 | 17 | 24 |
| 207163_s_at | 3217 | 3 | 10 |
| 207183_at | 230 | 6 | 6 |
| 207592_s_at | 350 | 5 | 14 |
| 207614_s_at | 2139 | 2 | 6 |
| 207622_s_at | 882 | 2 | 16 |
| 207828_s_at | 997 | 3 | 3 |
| 208002_s_at | 3142 | 3 | 7 |
| 208089_s_at | 1374 | 3 | 6 |
| 208308_s_at | 12282 | 4 | 9 |
| 208540_x_at | 5257 | 2 | 2 |
| 208644_at | 2242 | 2 | 3 |
| 208657_s_at | 1547 | 2 | 4 |
| 208677_s_at | 5414 | 3 | 14 |
| 208696_at | 7351 | 4 | 3 |
| 208710_s_at | 1112 | 3 | 44 |
| 208723_at | 4402 | 3 | 6 |
| 208744_x_at | 1673 | 4 | 49 |
| 208837_at | 3997 | 2 | 3 |
| 208916_at | 1630 | 3 | 5 |
| 208928_at | 1439 | 4 | 7 |
| 208956_x_at | 7772 | 3 | 2 |
| 208974_x_at | 6025 | 2 | 6 |
| 208975_s_at | 1085 | 2 | 3 |
| 209015_s_at | 1739 | 4 | 4 |
| 209036_s_at | 8944 | 3 | 3 |
| 209053_s_at | 269 | 7 | 10 |
| 209072_at | 6299 | 4 | 18 |
| 209079_x_at | 12870 | 3 | 3 |
| 209081_s_at | 3160 | 3 | 2 |
| 209123_at | 4686 | 4 | 3 |
| 209132_s_at | 4385 | 5 | 12 |
| 209172_s_at | 268 | 3 | 3 |
| 209197_at | 820 | 3 | 3 |
| 209198_s_at | 491 | 3 | 3 |
| 209247_s_at | 1486 | 2 | 2 |
| 209254_at | 1384 | 4 | 7 |
| 209255_at | 4283 | 6 | 8 |
| 209256_s_at | 4949 | 8 | 6 |
| 209283_at | 12529 | 5 | 3 |
| 209345_s_at | 1678 | 2 | 2 |
| 209407_s_at | 1461 | 2 | 6 |
| 209515_s_at | 5827 | 5 | 15 |
| 209773_s_at | 1243 | 3 | 5 |
| 209825_s_at | 765 | 2 | 3 |
| 209827_s_at | 4884 | 7 | 7 |
| 209828_s_at | 1146 | 4 | 5 |
| 209848_s_at | 32959 | 7 | 74 |
| 209875_s_at | 3038 | 21 | 12 |
| 209932_s_at | 7126 | 3 | 5 |
| 210052_s_at | 1085 | 3 | 6 |
| 210073_at | 337 | 4 | 6 |
| 210111_s_at | 7841 | 5 | 3 |
| 210127_at | 391 | 2 | 9 |
| 210854_x_at | 2100 | 2 | 6 |
| 210926_at | 574 | 2 | 3 |
| 210948_s_at | 396 | 2 | 4 |
| 210951_x_at | 2501 | 2 | 13 |
| 211013_x_at | 498 | 8 | 13 |
| 211052_s_at | 1399 | 3 | 2 |
| 211066_x_at | 12431 | 2 | 2 |
| 211373_s_at | 2063 | 5 | 6 |
| 211564_s_at | 1992 | 3 | 2 |
| 211752_s_at | 2183 | 2 | 2 |
| 211759_x_at | 5674 | 2 | 3 |
| 211833_s_at | 502 | 2 | 28 |
| 212000_at | 339 | 3 | 14 |
| 212070_at | 13437 | 2 | 4 |
| 212081_x_at | 1457 | 2 | 4 |
| 212119_at | 4415 | 2 | 5 |
| 212178_s_at | 2976 | 3 | 14 |
| 212193_s_at | 3646 | 3 | 10 |
| 212247_at | 1716 | 3 | 3 |
| 212285_s_at | 4252 | 2 | 4 |
| 212312_at | 1234 | 4 | 2 |
| 212338_at | 1598 | 3 | 4 |
| 212402_at | 3019 | 3 | 4 |
| 212472_at | 1987 | 5 | 5 |
| 212473_s_at | 3747 | 5 | 4 |
| 212512_s_at | 1441 | 3 | 2 |
| 212520_s_at | 2188 | 2 | 4 |
| 212552_at | 2611 | 2 | 2 |
| 212715_s_at | 1085 | 3 | 4 |
| 212739_s_at | 2736 | 2 | 3 |
| 212744_at | 959 | 3 | 5 |
| 212745_s_at | 376 | 2 | 15 |
| 212793_at | 3123 | 4 | 5 |
| 212796_s_at | 2511 | 2 | 2 |
| 213002_at | 1439 | 2 | 3 |
| 213007_at | 968 | 3 | 4 |
| 213008_at | 1086 | 6 | 10 |
| 213096_at | 924 | 3 | 3 |
| 213131_at | 1392 | 3 | 3 |
| 213169_at | 4028 | 5 | 4 |
| 213215_at | 1926 | 4 | 3 |
| 213217_at | 5848 | 10 | 6 |
| 213241_at | 9479 | 4 | 23 |
| 213274_s_at | 18263 | 12 | 26 |
| 213275_x_at | 17604 | 7 | 3 |
| 213330_s_at | 1233 | 2 | 7 |
| 213333_at | 1845 | 2 | 3 |
| 213338_at | 932 | 6 | 4 |
| 213392_at | 1022 | 2 | 2 |
| 213474_at | 723 | 2 | 3 |
| 213496_at | 2322 | 3 | 7 |
| 213573_at | 1643 | 2 | 3 |
| 213587_s_at | 10416 | 18 | 9 |
| 213638_at | 1827 | 26 | 102 |
| 213670_x_at | 1959 | 2 | 4 |
| 213720_s_at | 2248 | 2 | 3 |
| 213746_s_at | 4187 | 3 | 14 |
| 213836_s_at | 2605 | 8 | 5 |
| 213895_at | 1279 | 3 | 4 |
| 213960_at | 11768 | 80 | 29 |
| 214023_x_at | 1602 | 7 | 9 |
| 214068_at | 2148 | 9 | 10 |
| 214104_at | 814 | 2 | 3 |
| 214201_x_at | 746 | 2 | 3 |
| 214581_x_at | 510 | 5 | 6 |
| 214614_at | 542 | 10 | 9 |
| 214632_at | 358 | 2 | 2 |
| 214656_x_at | 2977 | 2 | 2 |
| 214687_x_at | 26310 | 2 | 3 |
| 214708_at | 249 | 2 | 3 |
| 214710_s_at | 575 | 2 | 3 |
| 214714_at | 2366 | 4 | 9 |
| 214717_at | 471 | 4 | 4 |
| 214752_x_at | 6462 | 3 | 4 |
| 214778_at | 311 | 3 | 27 |
| 214841_at | 913 | 9 | 11 |
| 214893_x_at | 214 | 10 | 9 |
| 214896_at | 3071 | 8 | 11 |
| 215025_at | 2365 | 149 | 93 |
| 215115_x_at | 12421 | 34 | 15 |
| 215126_at | 4940 | 8 | 19 |
| 215155_at | 505 | 6 | 4 |
| 215311_at | 10093 | 86 | 30 |
| 215812_s_at | 1176 | 3 | 13 |
| 215836_s_at | 9406 | 3 | 3 |
| 216194_s_at | 5011 | 3 | 3 |
| 216973_s_at | 1732 | 6 | 4 |
| 217033_x_at | 10961 | 21 | 19 |
| 217104_at | 317 | 6 | 3 |
| 217226_s_at | 2191 | 3 | 3 |
| 217297_s_at | 838 | 3 | 21 |
| 217377_x_at | 12402 | 27 | 18 |
| 217419_x_at | 2742 | 3 | 5 |
| 217624_at | 349 | 21 | 20 |
| 217799_x_at | 1724 | 2 | 11 |
| 217827_s_at | 4762 | 2 | 2 |
| 217867_x_at | 9024 | 3 | 9 |
| 217871_s_at | 19519 | 3 | 11 |
| 217891_at | 1271 | 2 | 3 |
| 218009_s_at | 1557 | 4 | 3 |
| 218030_at | 1316 | 2 | 3 |
| 218074_at | 3594 | 2 | 4 |
| 218143_s_at | 4007 | 3 | 5 |
| 218151_x_at | 1384 | 2 | 2 |
| 218152_at | 1440 | 2 | 3 |
| 218161_s_at | 941 | 5 | 5 |
| 218175_at | 3563 | 3 | 2 |
| 218330_s_at | 3853 | 7 | 4 |
| 218349_s_at | 588 | 3 | 14 |
| 218359_at | 796 | 3 | 5 |
| 218376_s_at | 1931 | 4 | 4 |
| 218447_at | 2209 | 2 | 3 |
| 218542_at | 409 | 3 | 6 |
| 218564_at | 433 | 2 | 4 |
| 218608_at | 627 | 3 | 4 |
| 218678_at | 11356 | 14 | 20 |
| 218774_at | 1061 | 2 | 5 |
| 218786_at | 732 | 3 | 2 |
| 218824_at | 1351 | 3 | 5 |
| 218839_at | 1996 | 38 | 7 |
| 218856_at | 3199 | 5 | 4 |
| 218888_s_at | 906 | 6 | 5 |
| 218931_at | 911 | 3 | 2 |
| 218952_at | 2661 | 7 | 7 |
| 218956_s_at | 1840 | 4 | 3 |
| 218980_at | 1627 | 3 | 8 |
| 218996_at | 1859 | 4 | 6 |
| 219011_at | 113 | 4 | 3 |
| 219039_at | 1852 | 2 | 5 |
| 219040_at | 480 | 3 | 10 |
| 219041_s_at | 3435 | 4 | 2 |
| 219051_x_at | 1127 | 3 | 8 |
| 219066_at | 621 | 4 | 2 |
| 219143_s_at | 3618 | 9 | 13 |
| 219148_at | 426 | 2 | 3 |
| 219152_at | 365 | 13 | 10 |
| 219219_at | 859 | 2 | 3 |
| 219361_s_at | 1033 | 3 | 7 |
| 219372_at | 376 | 2 | 2 |
| 219408_at | 421 | 3 | 36 |
| 219478_at | 5485 | 80 | 18 |
| 219491_at | 547 | 3 | 12 |
| 219522_at | 822 | 4 | 3 |
| 219537_x_at | 411 | 3 | 6 |
| 219555_s_at | 402 | 12 | 21 |
| 219578_s_at | 1419 | 17 | 26 |
| 219634_at | 686 | 3 | 15 |
| 219637_at | 355 | 3 | 3 |
| 219703_at | 378 | 3 | 3 |
| 219742_at | 409 | 3 | 9 |
| 219895_at | 528 | 6 | 3 |
| 219933_at | 1399 | 2 | 2 |
| 220116_at | 748 | 5 | 8 |
| 220155_s_at | 5010 | 5 | 6 |
| 220178_at | 5915 | 9 | 15 |
| 220454_s_at | 581 | 2 | 2 |
| 220864_s_at | 8416 | 3 | 3 |
| 220948_s_at | 11794 | 2 | 3 |
| 220973_s_at | 497 | 4 | 3 |
| 220974_x_at | 2540 | 2 | 2 |
| 220980_s_at | 3598 | 3 | 2 |
| 221059_s_at | 2438 | 5 | 3 |
| 221483_s_at | 9194 | 3 | 3 |
| 221484_at | 3834 | 3 | 3 |
| 221538_s_at | 3971 | 3 | 3 |
| 221558_s_at | 2356 | 3 | 5 |
| 221577_x_at | 4897 | 28 | 38 |
| 221641_s_at | 1199 | 2 | 4 |
| 221688_s_at | 3740 | 2 | 4 |
| 221710_x_at | 728 | 2 | 2 |
| 221732_at | 931 | 3 | 2 |
| 221759_at | 1261 | 4 | 21 |
| 221797_at | 430 | 2 | 7 |
| 221799_at | 1601 | 5 | 3 |
| 221815_at | 3293 | 17 | 144 |
| 221882_s_at | 1144 | 5 | 6 |
| 221902_at | 2491 | 4 | 4 |
| 221909_at | 243 | 22 | 17 |
| 221962_s_at | 1132 | 2 | 6 |
| 222116_s_at | 4208 | 2 | 4 |
| 222153_at | 445 | 3 | 8 |
| 222155_s_at | 1264 | 3 | 10 |
| 222175_s_at | 2415 | 3 | 6 |
| 222196_at | 224 | 3 | 6 |
| 222199_s_at | 2152 | 2 | 3 |
| 222206_s_at | 383 | 4 | 12 |
| 222212_s_at | 3715 | 3 | 4 |
| 222231_s_at | 2724 | 3 | 2 |
| 222234_s_at | 754 | 4 | 12 |
| 222240_s_at | 1331 | 3 | 3 |
| 222294_s_at | 2193 | 3 | 5 |
| 32811_at | 4194 | 2 | 3 |
| 40560_at | 1629 | 3 | 5 |
| 44783_s_at | 8503 | 14 | 6 |
| 46665_at | 7835 | 4 | 3 |
| 55093_at | 3032 | 4 | 4 |
| 63825_at | 10096 | 14 | 74 |
| 87100_at | 737 | 6 | 70 |

We selected a short list of genes with at least 10-fold over-expression in melanoma as compared to the benign specimens. The complete array dataset has been submitted to the NCBI/Genbank GEO database (series entry pending).

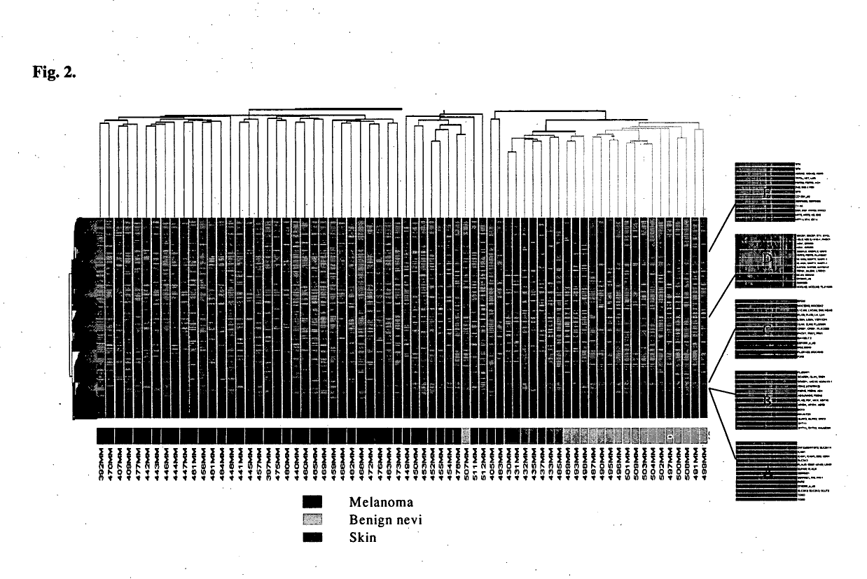

Hierarchical clustering revealed four distinct clusters (FIG. 2). Two clusters consisted of majority of the melanoma samples (43 out of 45); the third cluster included the majority of benign nevi samples (15 out of 18) and the fourth contained all 7 normal skin specimens. Melanoma samples themselves formed two clusters with 35 samples in one cluster and 10 samples in the other. Samples that formed the small cluster represented epithelioid melanoma only, visually contained less melanin and demonstrated higher expression of PRAME and MIA genes (p<0.05). The few stage III and IV tumors were all grouped in the small cluster. The large cluster showed higher expression of NTRK3 and nestin (NES) (p<0.05). All melanoma and benign nevi samples demonstrated equally high expression of known melanocyte markers such as tyrosinase and MART-I, confirming that there is comparable melanocyte content in these samples. Our data indicate that melanoma, benign nevi and normal skin samples have distinct gene expression profiles and can be separated on molecular basis. Selected genes that were highly expressed in melanoma and their associated functional categories are summarized in Table 9.

TABLE 9

| | | | | |
|---|---|---|---|---|
| | | Median | Median | |
| | | expression in | expression in | Fold |
| psid | Name | melanoma | benign/normal | change |

Neural system development and function

| | | | | |
|---|---|---|---|---|
| 215025_at | NTRK3 | 2365.1 | 19.9 | 118.8 |
| 204709_s_at | KNSL5 | 311.8 | 13.5 | 23.1 |
| 204585_s_at | L1CAM var 1 | 805.2 | 49 | 16.4 |
| 218678_at | NES | 11355.5 | 703.6 | 16.1 |
| 202260_s_at | STXBP1 | 7877.1 | 1312 | 6.0 |
| 204995_at | p35 | 496.3 | 89.2 | 5.6 |
| 208308_s_at | GP1 | 12281.7 | 2238.2 | 5.5 |
| 201340_s_at | ENC1 | 390.8 | 74.9 | 5.2 |
| 209072_at | MBP | 6299.1 | 1215.9 | 5.2 |

Cell movements

| | | | | |
|---|---|---|---|---|
| 214614_at | HOXB9 | 541.6 | 54.4 | 10.0 |
| 205447_s_at | MAP3K12 | 566.5 | 76.5 | 7.4 |

Tissue morphology

| | | | | |
|---|---|---|---|---|
| 206397_x_at | GDF1 | 1436.1 | 130.1 | 11.0 |
| 205458_at | MC1R | 3287.6 | 458.1 | 7.2 |

Cancer cell invasion

| | | | | |
|---|---|---|---|---|
| 213274_s_at | CSTTB | 18262.9 | 1261.4 | 14.5 |
| 208677_s_at | BSG | 5413.8 | 1088 | 5.0 |

Cell cycle control

| | | | | |
|---|---|---|---|---|
| 219578_s_at | CPEB1 | 1418.5 | 75.6 | 18.8 |
| 207144_s_at | CITED1 | 593.4 | 33.5 | 17.7 |
| 204252_at | CDK2 | 5293.7 | 869.3 | 6.1 |
| 211373_s_at | PSEN2 | 2063.3 | 403.2 | 5.1 |

Cell death

| | | | | |
|---|---|---|---|---|
| 221577_x_at | PLAB | 4896.9 | 173.9 | 28.2 |
| 205681_at | BCL2A1 | 1316.4 | 135.1 | 9.7 |

Unknown

| | | | | |
|---|---|---|---|---|
| 204545_at | PEX6 | 379.1 | 23.9 | 15.9 |
| 201850_at | CAPG | 11103.2 | 725.6 | 15.3 |
| 204014_at | DUSP4 | 7183.6 | 601.3 | 11.9 |
| 202779_s_at | E2-EPF | 3400 | 323.3 | 10.5 |
| 201954_at | ARPC1B | 25900.7 | 2470 | 10.5 |
| 209848_s_at | me20m | 32958.9 | 3778.4 | 8.7 |
| 213112_s_at | SQSTM1 | 260.4 | 33.9 | 7.7 |
| 218952_at | SAAS | 2660.7 | 368.7 | 7.2 |
| 204099_at | SMARCD3 | 2496.2 | 428.2 | 5.8 |
| 206999_at | IL12RB2 | 354 | 61.3 | 5.8 |
| 201251_at | PKM2 | 23964.7 | 4228.2 | 5.7 |
| 202185_at | PLOD3 | 8493.2 | 1541.7 | 5.5 |

### EXAMPLE 4

### Identification of Genes Differentially Expressed in Melanoma

A total of 70 gene expression profiles were used for analysis. The he median percentages of “present calls” for melanoma, benign and normal sample groups were 43.8%, 46.9% and 41.7%. Sixty microarrays (86%) had scaling factors within 3-fold range of the minimum value. Ten chips with the scaling factors more than 3 were equally distributed between the sample categories, melanoma, benign and normal.

Unsupervised hierarchical clustering result revealed a distinct separation of the melanoma, benign nevi and normal skin samples (FIG. 2). We observed four clusters, including two clusters consisting of majority of the melanoma samples (43 out of 45), the third cluster contained all 7 normal skin, 3 benign nevi and 2 melanoma specimens and the fourth cluster, that included 14 of the 18 benign nevi samples. Source of the samples did not affect clustering. Specimens originated from different sources were clustered together according the sample type (melanoma, benign or normal). To further test the stability of the clustering patterns, we used an alternative cut-off on gene filtering prior to the cluster analysis. Specifically, we retained genes that have at least 10% “present” calls in each of the melanoma, benign nevi and skin samples. With this cut-off, we obtained 15, 306 genes and repeated hierarchical clustering. The cluster pattern on the patient samples was the same as the one from the 15,795 from the 2 “present” calls, confirming clustering stability.

The single nevi sample that clustered with the melanoma samples is an atypical nevi (moderate degree) sample with no melanoma in-situ present. All three nevi samples that clustered with normal skin are compound nevi samples and one of them has melanocyte content lower than the other nevi specimens. The melanoma samples themselves formed two clusters with 34 samples in the large and 9 samples in the smaller cluster. Samples that formed the small cluster represented epithelioid melanoma only and visually contained less melanin. The few stage III and IV tumors, used in our study, were all grouped in the small cluster. The large cluster was composed from epithelioid, spindle cell and melanoma of mixed histology specimens with more significant presence of melanin. The large cluster included Stage I and Stage II specimens only.

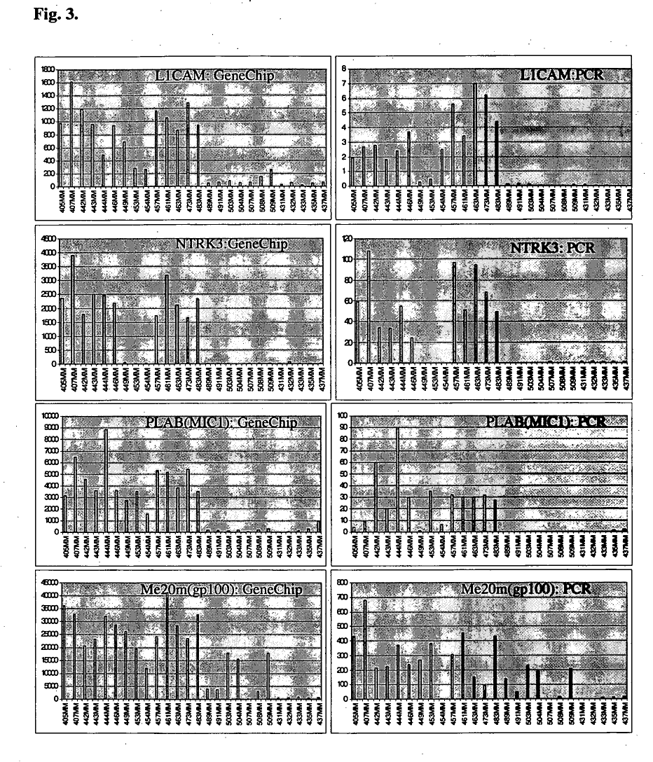

Distinct gene clusters were found in association to melanoma. This can be characterized by up-regulated (FIG. 2, A, B, C) and down-regulated (FIG. 2, E) genes in the melanoma samples. At the same time, melanoma and benign nevi samples demonstrated high expression of known melanocyte markers, such as MART-I (FIG. 3, D) confirming a comparable content of melanocyte in these samples and inability of melanocyte specific markers to differentiate them. Our data indicate that melanoma, benign nevi and normal skin samples have distinct gene expression profiles and can be separated on their molecular basis.

In order to identify genes upregulated in malignant melanoma, we applied SAM in combination with t-test with Bonferroni correction and percentile analysis (FIG. 1). Bonferroni-adjusted t-test and percentile analyses were used to address the multiple testing issue and the heterogeneity of the tumor samples, respectively. As the result of these analyses, 439 genes were selected and are summarized in Table 15 as SEQ ID NOs: 29-467. Out of 439 genes up-regulated in melanoma, we selected a short list of 33 genes that had more than 10-fold over-expression in the melanoma samples than that of the benign specimens. These include many genes with known association with malignant melanoma such as NTRK3 (Xu et al. (2003)), LlCAM (Fogel et al. (2003); and Thies et al. (2002)), me20m (Adema et al. (1994)), as well as novel genes. Genes with more than 10-fold overexpression in melanoma are presented in Table 10.

TABLE 10

| | | Median | Fold change | |
|---|---|---|---|---|
| | | Exp | (Can v | Fold change |
| Psid | Description | Melanoma | Benign) | (Can v skin) |
| 215025_at | NTRK3 | 2365 | 149 | 93 |
| 215311_at | EUROIMAGE | 10093 | 86 | 30 |
| | 21920 | | | |
| 213960_at | EUROIMAGE | 11768 | 80 | 29 |
| | 51358 | | | |
| 219478_at | WFDC1 | 5485 | 80 | 18 |
| 218839_at | HEY1 | 1996 | 38 | 7 |
| 215115_x_at | TEL oncogene | 12421 | 34 | 15 |
| 221577_x_at | PLAB | 4897 | 28 | 38 |
| 217377_x_at | ETV6-NTRK3 | 12402 | 27 | 18 |
| | fusion | | | |
| 213638_at | PHACTR1 | 1827 | 26 | 102 |
| 204709_s_at | KNSL5 | 312 | 24 | 16 |
| 221909_at | Hyp protein | 243 | 22 | 17 |
| | FLJ14627 | | | |
| 204584_at | L1CAM | 9677 | 21 | 15 |
| 209875_s_at | SPP1 | 3038 | 21 | 12 |
| 217624_at | PDAP1 | 349 | 21 | 20 |
| 203071_at | SEMA3B | 958 | 19 | 3 |
| 213587_s_at | C7ORF32 | 10416 | 18 | 9 |
| 221815_at | ABHD2 | 3293 | 17 | 144 |
| 219578_s_at | CPEB1 | 1419 | 17 | 26 |
| 207144_s_at | CITED1 | 593 | 17 | 24 |
| 203069_at | SV2A | 560 | 15 | 13 |
| 218678_at | NES | 11356 | 14 | 20 |
| 219152_at | PODXL2 | 365 | 13 | 10 |
| 205447_s_at | MAP3L12 | 567 | 13 | 5 |
| 213274_s_at | CTSB | 18263 | 12 | 26 |
| 219555_s-at | BMO39 | 402 | 12 | 21 |
| 203827_at | WIPI49 | 3380 | 11 | 8 |
| 205813_s_at | MAT1A | 430 | 11 | 9 |
| 201850_at | CAPG | 11103 | 10 | 20 |
| 205373_at | CTNNA2 | 579 | 10 | 8 |
| 214614_at | HLXB9 | 542 | 10 | 9 |
| 213217 | ADCY2 | 5848 | 10 | 6 |
| 204014_at | DUSP4 | 7184 | 10 | 46 |
| 214893 | HCN2 | 214 | 10 | 9 |

We further selected three genes over-expressed in melanoma, including NTRK3, PLAB, LlCAM, for quantitative real-time RT-PCR validation of the microarray results (FIG. 3). PLAB is a novel gene, whose differential expression in melanoma was not reported before at our best knowledge. For LlCAM and NTRK3, differential expression in melanoma was demonstrated at protein level only. Xu et al. (2003); Fogel et al. (2003); and Thies et al. (2002). Moreover, we identified PLAB and LlCAM as the best combination, on complementary basis, to separate melanoma from benign/normal tissues in our study. GPlOO is known as a melanoma specific marker and was selected as positive control. For the RT-PCR assay we used a panel of 14 primary melanoma, 7 benign nevi and 5 normal skin samples, isolated from the same tissues as used for the microarray study. The expression value of each gene was normalized to the housekeeping control gene PBGD. The correlation coefficients between the RT-PCR and the microarray results for LlCAM, NTRK3, PLAB and gplOO are 0.79, 0.86, 0.87 and 0.88, respectively. This result indicates that the RT-PCR results are highly consistent with the microarray data.

### EXAMPLE 5

### Pathway Analysis of Differentially Expressed Genes

Functional analysis of genes differentially expressed in melanoma was performed using Ingenuity™ Pathway Analysis Software Application (Ingenuity, Mountain View, Calif.). Functional categories or canonical pathways that have p-value of less than 0.05 were selected. Specificity of canonical pathways identification was tested using randomly selected genes.

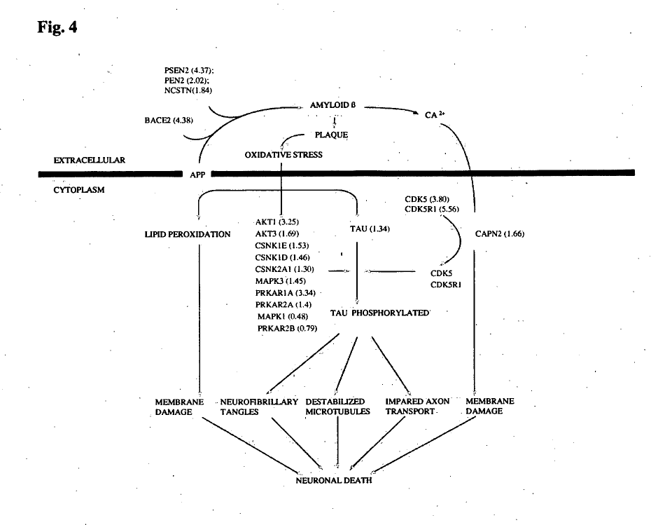

In order to gain further insight into a potential mechanism that differentiates melanoma from benign and normal tissue, we used Ingenuity pathway analysis software to identify canonical pathways associated with melanoma. The results analysis revealed that many of the genes in amyloid processing were up-regulated in the melanoma samples. To verify specificity of our observation, we selected three random lists of genes from Affymetrix HuI 33A microarray and subjected them to Ingenuity pathway analysis. None of these lists produced a significant association to amyloid processing or any other canonical pathways. To confirm the activation of this canonical pathway in melanoma, gene expression data for all the genes in the pathway were retrieved. Fold-change and p-value of differential expression between melanoma and benign/normal tissues were calculated. Out of the 34 genes included in the amyloid processing pathway (Esler et al. (2001); and Giancotti et al. (1999)), 25 demonstrated up-regulation trend and for 19 of them (56%), differential expression was statistically significant (p-value<0.05; FIG. 4). As the additional control, we randomly selected two metabolic pathways with a similar number of genes. Out of the 63 genes in alanine synthesis pathway, 8 of them (13%) showed significant up-regulation with p-value less than 0.05. Out of the 47 genes in histidine synthesis pathway, only 2 genes (4%) were found using the same criteria. For the first time, our data strongly indicated that activation of the amyloid processing pathway is involved in malignant melanoma.

### EXAMPLE 6

### RT-PCR Validation of Microarray Results

Ten microgram total RNA from each sample was treated with DNase I and reverse-transcribed with oligo (dT) primer using Superscript II reverse transcriptase according to the manufacturer's instructions (Invitrogen, Carlsbad, Calif.). A control gene PBGD was previously tested and reported as a housekeeping gene. Vandesompele et al. (2003). Primers and MGB-probes for me20m (gplOO), LlCAM, NTRK3, and the control gene PBGD were designed using Primer Express software (Applied Biosystems, Foster City, Calif.). The PLAB (MICl) gene probe was FAM-TAMRA based since sequences were inadequate to design MGB based probes. Primer/probe sequences were as follows:

TABLE 11

| | | |
|---|---|---|
| | | SEQ |
| | | ID |
| Description | Sequence | NO: |

| | | |
|---|---|---|
| me20m forward | TGTGTCTCTGGCTGATACCAACA | 983 |
| me20m reverse | TTCTTGACCAGGCATGATAAGCT | 984 |
| me20m probe | (6-FAM) CTGGCAGTGGTCAGC | 985 |
| L1CAM forward | GCTGGGACTGGGAACAGAACT | 10 |
| L1CAM reverse | GGAGCAGAGATGGCAAAGAAA | 11 |
| LICAM probe | (6-FAM) TCCCCACCATCTGCTGT | 12 |
| NTRK3 forward | GCCCCGGCACCCTTTA | 16 |
| NTRK3 reverse | AACCCTGCCAGTGGTGGAT | 17 |
| NTRK3 probe | (6-FAM) CAGATGGGTGTTTTC | 18 |
| PLAB forward | GGCAGAATCTTCGTCCGCA | 4 |
| PLAB reverse | GGACAGTGGTCCCCGTTG | 5 |
| PLAB probe | (6-FAM) CCCAGCTGGAGTTGCACTTGCGGCC(TAMRA) | 6 |
| PBGD forward | CTGCTTCGCTGCATCGCTGAAA | 986 |
| PBGD reverse | CAGACTCCTCCAGTCAGGTACA | 987 |
| PBGD probe | (6-FAM) CCTGAGGCACCTGGAAGGAGGCTGCAGTGT(TAMRA) | 988 |

All primers and probes were tested for optimal amplification efficiency above 90%. The standard curve was composed of six 10-fold dilutions of target gene PCR product with copy numbers ranging from 10 to 10⁶. RT-PCR amplification was carried out in a 20 μl reaction mix containing 50 ng template cDNA, 2× TaqMan® universal PCR master mix (12.5 μl) (Applied Biosystems, Foster City, Calif.), 50 OnM forward and reverse primers, and 25 OnM probe. Reactions were run on an ABI PRISM 7900HT Sequence Detection System (Applied Biosystems, Foster City, Calif.). The cycling conditions were: 2 min of AmpErase UNG activation at 50° C., 10 min of polymerase activation at 95° C. and 50 cycles at 95° C. for 15 sec and annealing temperature (60° C.) for 60 sec. In each assay, a standard curve and a no-template control along with template cDNA were included in duplicate for both the gene of interest and the control gene. The relative quantity of each target gene was represented as ΔCt, which is equal to Ct of the target gene subtracted by Ct of the control gene.

To confirm the melanoma specific genes identified by the microarray analysis, four genes (LlCAM, NTRK3, PLAB and gplOO) were selected for quantitative real-time RT-PCR validation (FIG. 4). The expression value of each gene was normalized to housekeeping control PBGD. The correlation coefficient between the RT-PCR and the microarray results for LlCAM, NTRK3, PLAB and gplOO are 0.79, 0.86, 0.87 and 0.88, respectively, indicating that the RT-PCR results are highly consistent with the microarray data.

### EXAMPLE 7

### One-Step qRTPCR Assays Using RNA-Specific Primers and Cutoff Establishment

Evaluation of expression of selected genes was carried out with one-step RT-PCR with RNA from primary melanoma, benign nevi, normal skin, melanoma IN metastasis and melanoma-free lymph nodes. Beta-actin was used as a housekeeping gene to control for the input quantity and quality of RNA in the reactions. DNase treatment was not used. Instead, primers or probes were designed to span an intron so they would not report on genomic DNA. Eight ng of total RNA was used for the RT-PCR. The Total RNA was reverse transcribed using 40× Multiscribe and RNase inhibitor mix contained in the TaqMan® One Step PCR Master Mix Reagents Kit (Applied Biosystems, Foster City, Calif.). The cDNA was then subjected to the 2× Master Mix without UNG and PCR amplification was carried out on the ABI 7900HT Sequence Detection System (Applied Biosystems, Foster City, Calif.) in the 384-well block format using a 10 μl reaction size. The primer and probe concentrations were 4 μM and 2.5 μM, respectively. The reaction mixture was incubated at 48° C. for 30 min for the reverse transcription, followed by a Amplitaq activation step of 95° C. for 10 min and finally 40 cycles of 95° C. for 15 sec denaturing and 60° C. for 1 min anneal and extension. On each plate a standard curve is generated from 8 pg to 80 ng and when the R2 value was greater than 0.99 the Cycle Threshold (Ct) values were accepted.

Sequences used in the reactions were as follows, each written in the 5′ to 3′ direction.

TABLE 12

| | | |
|---|---|---|
| | | SEQ |
| | | ID |
| Description | Sequence | NO: |

| | | |
|---|---|---|
| L1CAM Forward | CCACAGATGACATCAGCCTCAA | 13 |
| L1CAM Reverse | GGTCACACCCAGCTCTTCCTT | 14 |
| L1CAM probe | TGGCAAGCCCGAAGTGCAGTTCC | 15 |
| Tyrosinase | CTTTAGAAATACACTGGAAGGATTTGCTA | 1000 |
| Forward | | |
| Tyrosinase | CATTGTGCATGCTGCTTTGA | 1001 |
| Reverse | | |
| Tyrosinase probe | TCCACTTACTGGGATAGCGGATGCCTC | 1002 |
| MART1 Forward | ACTTCATCTATGGTTACCCCAAGAA | 1003 |
| MART1 Reverse | TCCCAGCGGCCTCTTCA | 1004 |
| MART1 Probe | CACGGCCACTCTTACACCACGGC | 1005 |
| HMB45 Forward | CTTAAGGCTGGTGAAGAGACAAGTC | 1006 |
| gp100 Reverse | CAGGATCTCGGCACTTTCAATAC | 1007 |
| gp100 Probe | TCGATATGGTTCCTTTTCCGTCACCCTG | 1008 |
| PLAB Forward | ATTCGAACACCGACCTCGTC | 1009 |
| PLAB Reverse | CGCAGGTGCAGGTGGC | 1010 |
| PLAB Probe | GATACTCACGCCAGAAGTGCGGCT | 1011 |

For each sample ΔCt=Ct (Target Gene)—Ct β-actin was calculated. ΔCt has been widely used in clinical RT-PCR assays and was chosen as a straightforward method. Cronin et al. (2004). T-test was performed on ΔCt between the melanoma and non-melanoma samples including both primary and LN samples. We then used ΔCt to construct two scores for each patient. One score was derived from a combination of 2 melanoma specific genes, PLAB and LlCAM; and the other score was derived from a combination of 3 conventional melanoma markers, tyrosinase, gplOO and MARTl. The score was defined as the weighted sum of ΔCt values of the tested genes with the corresponding t statistics as the weight. The two scores were normalized to have the same mean in order to compare them on the same scale.

We examined a combination of two highly overexpressed in melanoma genes, PLAB and LlCAM, in a variety of clinical tissue samples containing malignant melanocytes (primary melanoma and melanoma LN metastasis), benign melanocytes (benign skin nevi) and normal samples (normal skin and melanoma-free LN) by RT-PCR. The primary tissues were the same as those used for the microarray study while all the LN specimens were derived from independent patients. Conventional melanoma markers, such as tyrosinase, gplOO and MARTI, were also tested on the same samples as the controls because they are the most commonly used markers for the melanoma molecular assays in current clinical studies. Rimboldi et al. (2003); Abrahamsen et al. (2005); and Kammula et al. (2004). Calculated scores were presented on FIG. 4A for PLAB and LlCAM and on FIG. 4B for tyrosinase, gplOO and MARTl. The results demonstrated significant difference in expression of PLAB and LlCAM between malignant melanoma samples (primary and LN metastasis) and benign nevi and normal LN. In contrast, three conventional markers showed similar expression levels in benign and melanoma samples. To further demonstrate the ability of gene markers to separate benign and malignant tissues, we tested two cut-offs; first was set up as the highest score in primary normal samples and the second as the highest score in benign nevi samples. For each cut-off we estimated sensitivity and of the assay in the LN samples. With the cut-off determined on the normal samples, the new markers and the conventional markers gave sensitivity of 90% and 83%, respectively. Using the cut-off determined on the benign samples, the sensitivity for the new and conventional markers were 88% and 42%. The results indicated that the new markers potentially have better abilities to differentiate tissues containing benign and malignant melanocytes.

### EXAMPLE 8

### Multiplex Assay

Materials and Methods

Each reaction was set up in a final volume of 25 μl containing the following:

| | |
|---|---|
| forward primer | 40O nM |
| reverse primer | 50O nM |
| PLAB probe | 15O nM |
| Tyrosinase probe | 30O nM |
| L1CAM probe | 20O nM |
| PBGD probe | 20O nM |
| Tth | 5 U |
| Ab TP 6-25 | i μg |
| Glycerol | 10% |
| Tris-HCl | 3J mM |
| NaCl | 4 mM |
| EDTA | 0.004 mM |
| Tween-20 | 0.22% |
| NP-40 | 0.02% |
| DTT | 0.04 mM |
| Potassium Hydroxide | 20.5 mM |
| Bicine | 5O mM |
| Potassium Acetate | 115 mM |
| Albumin, bovine | 5 μg |
| Trehalose | 0.15 M |
| dNTP | 0.2 mM ea |
| MgCl₂ | 0.5 mM |
| MnSO₄ | 3.5 mM |
| Primers | 300 nM ea |
| Probes | 200 nM ea |

The primer and probe sequences are provided in Table 13.

TABLE 13

| | | |
|---|---|---|
| SEQ | | |
| ID NO | Sequence 5′-3′ | Function |

| | | |
|---|---|---|
| 43 | gaacaccgacctcgtccc | PLAB Upper Primer |
| 44 | ggcggcccgagagata | PLAB Lower Primer |
| 45 | Fam-cgccagaagtgcggctgggat-BHQ1-tt | PLAB Probe |
| 55 | actcagcccagcatcattcttc | Tyr Upper Primer |
| 56 | atggctgttgtactcctccaatc | Tyr Lower Primer |
| 57 | Q570-cttctcctcttggcagattgtctgtagc BHQ2-tt | Tyr Probe |
| 49 | ccacagatgacatcagcctcaa | L1CAM Upper Primer |
| 50 | ggtcacacccagctcttcctt | L1CAM Lower Primer |
| 51 | Ca1Red-tggcaagcccgaagtgcagttcc-BHQ2-tt | L1CAM Probe |
| 58 | ccacacacagcctactttccaa | PBGD Upper Primer |
| 59 | tacccacgcgaatcactctca | PBGD Lower Primer |
| 60 | Q670-aacggcaatgcggctgcaacggcggaa-BHQ2-tt | PBGD Probe |

The reactions are run with PLAB in Fam, Tyrosinase in Cy3, LlCAM in Texas Red, and PBGD in Cy5 channels. The cycling protocol used is described below and takes 30 min to complete.
95° C.×15 sec
65° C.×420 sec
40° cycles of:
95° C. for 5 sec
62° C. for 15 sec—fiuor read

The thresholds used are 30 in Fam, 20 in Cy3, 20 in Texas Red, and 20 in Cy5 channels. The thresholds employed in the Cy3 and Texas red channels can be lowered. The results obtained are summarized in Table 14.

TABLE 14
Best Marker Combinations

| | % Sensitivity | |
|---|---|---|
| Markers | (95% CI) | % Specificity (95% CI) |
| L1CAM + PLAB | 82 (73-89) | 96 (87-100) |
| Tyrosinase + ME20M (GP100) | 63 (52-72) | 100 (94-100) |
| L1CAM + PLAB + Tyrosinase | 87 (79-93) | 96 (87-100) |

Ct Cutoffs:

| | |
|---|---|
| L1CAM | 27 |
| PLAB | 29 |
| Tyrosinase | 23 |
| ME20M (GP1OO) | 23.5 |

Note: these data are benchmarked against H&E pathology only. The amplification efficiency in each of the 4 reactions is high and the reaction is also linear over 5 logs (as judged by the R2 value which is >0.99 in all cases). Therefore, these data demonstrate a working 4 plex, rapid assay. These data suggest that PLAB is the primary marker and complementation, achieved with LlCAM, further increases sensitivity. If required, addition of tyrosinase as a third marker further complements LlCAM and PLAB and increases sensitivity. Tyrosinase can be dropped from the assay, if needed, without affecting the performance of the remaining markers.

Discussion

We performed gene expression profiling analysis of primary melanoma, benign nevi and normal skin tissue specimens in order to find melanoma specific gene markers for potential use in the LN molecular staging assay. Novel genes that are highly and differentially expressed in malignant melanoma samples were identified. Inclusion of benign nevi in the experimental design was key to our study. In contrast to normal skin, melanocyte content in benign nevi is close to that in melanoma. This was confirmed, in addition to histological assessment, by equally high expression level of conventional melanoma markers such as tyrosinase and MARTl in both melanoma and nevi tissue specimens. Similar cellular composition allowed us to monitor gene expression changes specifically associated with melanocyte malignant transformation, not just with melanocyte lineage differentiation. As the result, we identified novel genes specifically overexpressed in melanoma. One of the novel highly overexpressed in melanoma genes, prostate differentiation factor (PLAB, MICl), is a member of transforming growth factor-beta superfamily and also known to be associated with other malignancies. Bae et al. (2003); and Welsh et al. (2003). PLAB reduces cell adhesion (Yamauchi et al. (2003)), implicating its potential role in melanoma progression. Pathway analysis of the overexpressed genes in melanoma indicated that many of these genes belong to neural tissue functioning and development, suggesting that dedifferentiation of melanocytes and activation of the processes related to a pluripotent progenitor cell might be important for melanoma development and progression. Moreover, the analysis of canonical pathways showed that neural tissue associated amyloid processing is significantly modulated in melanoma. Amyloid processing (APP) pathway itself has not been associated with melanoma development and progression before. Many genes in the APP pathway, such as members of the β- and Y-secretase family (BACE2, PSEN2) also participate in the Notch pathway and play a role of cleavage of integral membrane proteins in both Notch and APP. Esler et al. (2001). Notch suppresses differentiation and helps maintain neural crest stem cells in undifferentiated state (Gangemi et al. (2004)) and Notch's involvement in melanoma and, particularly, the role of Y-secretases is the focus of many studies. Hoek et al. (2004); Baldi et al. (2003); and Wilson et al. (2000).

We have compared our results to the recent study of Haqq et al (2005). In their work, cDNA microarray containing 20,862 probes was used to profile benign nevi, primary melanoma and metastatic melanoma specimens. The sample set included metastatic and primary melanoma and benign nevi. Similar clustering results that separated the benign nevi and the primary malignant melanoma tissues were found in their study. Common genes were reported in both studies that can discriminate melanoma from benign nevi including kinesin-like 5 (KNSL5), prostate differentiation factor (PLAB), CITEDl, osteopontin (SPPl), cathepsin B (CSTB), cadherin 3 (CDH3), presenilin 2 (PSEN2).

Our results of the one-step RT-PCR assay demonstrated that novel melanoma specific gene PLAB and LlCAM expressed not only in primary melanoma tissues but also in melanoma LN metastasis. Moreover, the ability to differentiate malignant melanoma from benign nevi made them better candidates than the conventional markers for the molecular test of melanoma diagnostics. With further validation in clinical studies, these genes could be developed as specific markers for a molecular staging assay to detect melanoma micrometastasis during sentinel lymph node (SLN) biopsy procedure. Another potential application of the genes is for diagnosis of melanocyte lesions with uncertain pathological features.

Although the foregoing invention has been described in some detail by way of illustration and example for purposes of clarity of understanding, the descriptions and examples should not be construed as limiting the scope of the invention.

TABLE 15
Sequence Descriptions, names and SEQ ID NOS:

| | | | | |
|---|---|---|---|---|
| 1 | | | PLAB | |
| 2 | | | L1CAM | |
| 3 | | | NTRK | |
| 4 | | | | PLAB forward primer |
| 5 | | | | PLAB reverse primer |
| 6 | | | | PLAB probe |
| 7 | | | | PLAB upper primer |
| 8 | | | | PLAB lower primer |
| 9 | | | | PLAB probe |
| 10 | | | | L1CAM forward primer |
| 11 | | | | L1CAM reverse primer |
| 12 | | | | L1CAM probe |
| 13 | | | | L1CAM upper primer |
| 14 | | | | L1CAM lower primer |
| 15 | | | | L1CAM probe |
| 16 | | | | NTRK primer |
| 17 | | | | NTRK primer |
| 18 | | | | NTRK probe |
| 19 | | | | Tyrosinase upper primer |
| 20 | | | | Tyrosinase lower primer |
| 21 | | | | Tyrosinase probe |
| 22 | | | | PBGD upper primer |
| 23 | | | | PBGD lower primer |
| 24 | | | | PBGD probe |
| 25 | | | | PLAB amplicon |
| 26 | | | | L1CAM amplicon |
| 27 | | | | Tyrosinase amplicon |
| 28 | | | | PBGD amplicon |
| 29 | 200078_s_at | BC005876 | | ATPase, H+ transporting, lysosomal |
| 30 | 200601_at | U48734 | | non-muscle alpha-actinin |
| 31 | 200612_s_at | NM_001282 | AP2B1 | adaptor-related protein complex 2, β 1 |
| 32 | 200644_at | NM_023009 | MACMARCKS | macrophage myristoylated alanine- |
| | | | | rich C kinase substrate |
| 33 | 200660_at | NM_005620 | S100A11 | S100 calcium-binding protein A11 |
| 34 | 200707_at | NM_002743 | PRKCSH | protein kinase C substrate 80K-H |
| 35 | 200736_s_at | NM_000581 | GPX1 | glutathione peroxidase 1 |
| 36 | 200737_at | NM_000291 | PGK1 | phosphoglycerate kinase 1 |
| 37 | 200783_s_at | NM_005563 | LAP 18 | leukemia-assoc phosphoprotein p18 |
| 38 | 200825_s_at | NM_006389 | ORP150 | oxygen regulated protein (15O kD) |
| 39 | 200827_at | NM_000302 | PLOD | procollagen-lysine, 2-oxoglutarate 5- |
| | | | | dioxygenase |
| 40 | 200837_at | NM_005745 | DXS1357E | accessory proteins BAP31BAP29 |
| 41 | 200838_at | NM_001908 | CTSB | cathepsin B |
| 42 | 200839_s_at | NM_001908 | CTSB | |
| 43 | 200859_x_at | NM_001456 | FLNA | filamin A, alpha |
| 44 | 200910_at | NM_005998 | CCT3 | chaperonin containing TCP1, sub 3(γ) |
| 45 | 200950_at | NM_006409 | ARPC1A | actin related protein 23 complex, sub |
| | | | | 1A |
| 46 | 200950_at | NM_006409 | ARPC1A | |
| 47 | 200966_x_at | NM_000034 | ALDOA | aldolase A, fructose-bisphosphate |
| 48 | 200967_at | NM_000942 | PPiB | peptidylprolyl isomerase B |
| 49 | 200968_s_at | NM_000942 | PPIB | |
| 50 | 200972_at | BC000704 | | tetraspan 3 |
| 51 | 201038_s_at | BE560202 | | putative HLA class II assoc protein I |
| 52 | 201051_at | BE560202 | | putative HLA class II assoc protein I |
| 53 | 201105_at | NM_002305 | LGALS1 | lectin, galactoside-binding, soluble, 1 |
| 54 | 201106_at | NM_002085 | GPX4 | glutathione peroxidase 4 |
| 55 | 201188_s_at | D26351 | ITPR3 | type 3 inositol 1,4,5-trisphosphate |
| | | | | receptor |
| 56 | 201189_s_at | NM_002224 | ITPR3 | |
| 57 | 201195_s_at | AB018009 | | L-type amino acid transporter 1 |
| 58 | 201202_at | NM_002592 | PCNA | proliferating cell nuclear antigen |
| 59 | 201251_at | NM_002654 | PKM2 | pyruvate kinase, muscle |
| 60 | 201252_at | NM_006503 | PSMC4 | proteasome 26S subunit, ATPase, 4 |
| 61 | 201271_s_at | NM_016732 | RALY | RNA-binding protein transcript var 1 |
| 62 | 201291_s_at | NM_001067 | | topoisomerase (DNA) II alpha |
| 63 | 201313_at | NM_001975 | ENO2 | enolase 2 |
| 64 | 201346_at | NM_024551 | FLJ21432 | hypothetical protein FLJ21432 |
| 65 | 201393_s_at | NM_000876 | IGF2R | insulin-like growth factor 2 receptor |
| 66 | 201416_at | NM_003107 | | SRY-box 4 |
| 67 | 201417_at | NM_003107 | | SRY-box 4 |
| 68 | 201470_at | NM_004832 | GSTTLp28 | glutathione-S-transferase like; |
| | | | | glutathione transferase omega |
| 69 | 201474_s_at | NM_002204 | ITGA3 | integrin, alpha 3 transcript variant a |
| 70 | 201485_s_at | BC004892 | RCN2 | reticulocalbin 2, EF-hand calcium |
| | | | | binding domain |
| 71 | 201486_at | NM_002902 | RCN2 | |
| 72 | 201536_at | AL048503 | | DKFZp586M1524 |
| 73 | 201614_s_at | NM_003707 | RUVBL1 | RuvB (E coli homolog)-like 1 |
| 74 | 201660_at | | FACL3 | fatty-acid-Coenzyme A ligase, long- |
| | | | | chain 3 |
| 75 | 201661_s_at | NM_004457 | FACL3 | |
| 76 | 201662_s_at | D89053 | | Acyl-CoA synthetase 3 |
| 77 | 201670_s_at | M68956 | MARCKS 80K-L | myristoylated alanine-rich C-kinase |
| | | | | substrate |
| 78 | 201714_at | NM_001070 | TUBG1 | tubulin, gamma 1 |
| 79 | 201765_s_at | AL523158 | | hexosaminidase A |
| 80 | 201792_at | NM_001129 | AEBP1 | AE-binding protein 1 |
| 81 | 201804_x_at | NM_001281 | CKAP1 | cytoskeleton-associated protein 1 |
| 82 | 201819_at | NM_005505 | CD36L1 | CD36 antigen-like 1 |
| 83 | 201850_at | NM_001747 | CAPG | capping protein gelsolin-like |
| 84 | 201880_at | NM_005744 | | ariadne (Drosophila) homolog, |
| | | | | ubiquitin-conjugating enzyme E2- |
| | | | | binding protein, 1 |
| 85 | 201910_at | BF213279 | FARP1 | RhoGEF & pleckstrin domain 1 |
| 86 | 201911_s_at | NM_005766 | FARP1 | |
| 87 | 201931_at | NM_000126 | ETFA | electron-transfer-flavoprotein, α |
| | | | | polypeptide |
| 88 | 201954_at | NM_005720 | ARPC1B | actin related protein 23 com, sub 1A |
| 89 | 201976_s_at | NM_012334 | MYO10 | myosin X |
| 90 | 202069_s_at | AI826060 | IDH3A | isocitrate dehydrogenase 3 alpha |
| 91 | 202070_s_at | NM_005530 | IDH3A | |
| 92 | 202111_at | NM_003040 | SLC4A2 | solute carrier fam 4 anion exchanger |
| | | | | mem 2 |
| 93 | 202154_x_at | NM_006086 | TUBB4 | tubulin, beta, 4 |
| 94 | 202185_at | NM_001084 | PLOD3 | procollagen-lysine, 2-oxoglutarate 5- |
| | | | | dioxygenase |
| 95 | 202188_at | NM_014669 | KIAA0095 | KIAA0095 gene product |
| 96 | 202219_at | NM_005629 | SLC6A8 | solute carrier family 6, member 8 |
| 97 | 202224_at | NM_016823 | | v-crk avian sarcoma virus CT10 |
| | | | | oncogene homolog |
| 98 | 202225_at | NM_016823 | | v-crk avian sarcoma virus CT10 |
| | | | | oncogene homolog |
| 99 | 202260_s_at | NM_003165 | STXBP1 | syntaxin binding protein 1 |
| 100 | 202295_s_at | NM_004390 | CTSH | cathepsin H |
| 101 | 202329_at | NM_004383 | CSK | c-src tyrosine kinase |
| 102 | 202367_at | NM_001913 | CUTL1 | cut (Drosophila)-like 1 |
| 103 | 202370_s_at | NM_001755 | CBFB | core-binding factor, β sub trans var 2 |
| 104 | 202478_at | NM_021643 | GS3955 | GS3955 protein |
| 105 | 202503_s_at | NM_014736 | KIAA0101 | KIAA0101 gene product |
| 106 | 202589_at | NM_001071 | TYMS | thymidylate synthetase |
| 107 | 202603_at | N51370 | | disintegrin and metalloproteinase |
| | | | | domain 10 |
| 108 | 202705_at | NM_004701 | CCNB2 | cyclin B2 |
| 109 | 202737_s_at | NM_012321 | LSM4 | U6 snRNA-associated Sm-like protein |
| 110 | 202779_s_at | NM_014501 | E2-EPF | ubiquitin carrier protein |
| 111 | 202785_at | NM_005001 | NDUFA7 | NADH dehydrogenase 1 α |
| | | | | subcomplex, 7 |
| 112 | 202862_at | NM_000137 | FAH | fumarylacetoacetate |
| 113 | 202898_at | NM_014654 | KIAA0468 | KIAA0468 gene product |
| 114 | 202954_at | NM_007019 | UBCH10 | ubiquitin carrier protein E2-C |
| 115 | 202958_at | NM_002833 | PTPN9 | protein tyrosine phosphatase, non- |
| | | | | receptor type 9 |
| 116 | 202961_s_at | NM_004889 | ATP5J2 | ATP synthase, H+ transporting, |
| | | | | mitochondrial F0 complex, subunit f, |
| | | | | isoform 2 |
| 117 | 202986_at | NM_014862 | KIAA0307 | KIAA0307 gene product |
| 118 | 203011_at | NM_005536 | IMPA1 | inositol(myo)-1(or 4)- |
| | | | | monophosphatase 1 |
| 119 | 203022_at | NM_006397 | RNASEHI | ribonuclease HI, large subunit |
| 120 | 203069_at | NM_014849 | KIAA0736 | KIAA0736 gene product |
| 121 | 203071_at | NM_004636 | SEMA3B | sema domain, Ig domain, short basic |
| | | | | domain, secreted, 3B |
| 122 | 203094_at | NM_014628 | KIAA0110 | gene predicted from cDNA |
| 123 | 203145_at | NM_006461 | DEEPEST | mitotic spindle coiled-coil related |
| 124 | 203167_at | NM_003255 | TIMP2 | tissue inhibitor of metalloproteinase 2 |
| 125 | 203217_s_at | NM_003896 | SIAT9 | sialyltransferase 9 |
| 126 | 203234_at | NM_003364 | UP | uridine phosphorylase |
| 127 | 203256_at | NM_001793 | CDH3 | cadherin 3, type 1, P-cadherin |
| | | | | (placental) |
| 128 | 203262_s_at | NM_004699 | DXS9928E | chromosome X 9928 expressed seq |
| 129 | 203300_x_at | NM_003916 | AP1S2 | adaptor-related protein complex 1, |
| | | | | sigma 2 subunit |
| 130 | 203315_at | BC000103 | | NCK adaptor protein 2, |
| 131 | 203366_at | NM_002693 | POLG | polymerase (DNA directed), gamma |
| 132 | 203396_at | NM_002789 | PSMA4 | proteasome subunit, α type, 4 |
| 133 | 203452_at | NM_012200 | B3GAT3 | beta-1,3-glucuronyltransferase 3 |
| 134 | 203456_at | NM_007213 | JM4 | JM4 protein |
| 135 | 203502_at | NM_001724 | BPGM | 2,3-bisphosphoglycerate mutase |
| 136 | 203518_at | NM_000081 | CHS1 | Chediak-Higashi syndrome 1 |
| 137 | 203554_x_at | NM_004219 | PTTG1 | pituitary tumor-transforming 1 |
| 138 | 203557_s_at | NM_000281 | PCBD | 6-pyruvoyl-tetrahydropterin |
| | | | | synthasedimerization cofactor of |
| | | | | hepatocyte nuclear factor 1 alpha |
| 139 | 203570_at | NM_005576 | LOXL1 | lysyl oxidase-like 1 |
| 140 | 203590_at | NM_006141 | DNCLI2 | dynein, cytoplasmic, light intermediate |
| | | | | polypeptide 2 |
| 141 | 203643_at | NM_006494 | ERF | Ets2 repressor factor |
| 142 | 203663_s_at | NM_004255 | COX5A | cytochrome c oxidase subunit Va |
| 143 | 203668_at | NM_006715 | MAN2C1 | mannosidase, α, class 2C, mem 1 |
| 144 | 203693_s_at | NM_001949 | E2F3 | E2F transcription factor 3 |
| 145 | 203695_s_at | NM_004403 | DFNA5 | deafness, autosomal dominant 5 |
| 146 | 203723_at | NM_002221 | ITPKB | inositol 1,4,5-trisphosphate 3-kinase B |
| 147 | 203729_at | NM_001425 | EMP3 | epithelial membrane protein 3 |
| 148 | 203730_s_at | BF196931 | ZFP95 | zinc finger protein homologous to |
| | | | | Zfp95 in mouse |
| 149 | 203731_s_at | NM_014569 | ZFP95 | |
| 150 | 203775_at | NM_014251 | SLC25A13 | solute carrier family 25, member 13 |
| 151 | 203827_at | NM_017983 | FLJ10055 | hypothetical protein FLJ10055 |
| 152 | 203878_s_at | NM_005940 | MMP11 | matrix metalloproteinase 11 |
| 153 | 204014_at | NM_001394 | DUSP4 | dual specificity phosphatase 4 |
| 154 | 204015_s_at | BC002671 | DUSP4 | |
| 155 | 204033_at | NM_004237 | TRIP13 | thyroid hormone receptor interactor 13 |
| 156 | 204092_s_at | NM_003600 | STK15 | serinethreonine kinase 15 |
| 157 | 204099_at | NM_003078 | SMARCD3 | SWISNF related, matrix associated, |
| | | | | actin dependent regulator of |
| | | | | chromatin, subfamily d, member 3 |
| 158 | 204170_s_at | NM_001827 | CKS2 | CDC28 protein kinase 2 |
| 159 | 204197_s_at | NM_004350 | RUNX3 | runt-related transcription factor 3 |
| 160 | 204198_s_at | AA541630 | RUNX3 | |
| 161 | 204202_at | NM_017604 | KIAA1023 | KIAA1023 protein |
| 162 | 204228_at | NM_006347 | USA-CYP | cyclophilin |
| 163 | 204244_s_at | NM_006716 | ASK | activator of S phase kinase |
| 164 | 204247_s_at | NM_004935 | CDK5 | cyclin-dependent kinase 5 |
| 165 | 204252_at | M68520 | | cdc2-related protein kinase |
| 166 | 204262_s_at | NM_000447 | PSEN2 | presenilin 2 transcript variant 1 |
| 167 | 204423_at | NM_013255 | MKLN1 | muskelin 1, intracellular mediator |
| | | | | containing kelch motifs |
| 168 | 204436_at | NM_025201 | PP1628 | hypothetical protein PP1628 |
| 169 | 204458_at | AL110209 | | DKFZp564A0122 |
| 170 | 204467_s_at | NM_000345 | SNCA | synuclein, α transcript var NACP140 |
| 171 | 204584_at | AI653981 | L1CAM | L1 cell adhesion molecule, MASA |
| | | | | transcript var 1 |
| 172 | 204585_s_at | NM_000425 | L1CAM | |
| 173 | 204647_at | NM_004838 | HOMER-3 | Homer, neuronal imm early gene, 3 |
| 174 | 204654_s_at | NM_003220 | TFAP2A | transcription factor AP-2 alpha |
| 175 | 204709_s_at | NM_004856 | KNSL5 | kinesin-like 5 |
| 176 | 204778_x_at | AW102783 | HOXB7 | homeo box B7 |
| 177 | 204779_s_at | NM_004502 | HOXB7 | |
| 178 | 204857_at | NM_003550 | MAD1L1 | MAD1-like 1 |
| 179 | 204932_at | BF433902 | | TNF receptor superfam, mem 11b |
| 180 | 204973_at | NM_000166 | GJB1 | gap junction protein, beta 1, 32 kD |
| 181 | 204995_at | AL567411 | | cyclin-dependent kinase 5, regulatory |
| | | | | sub 1 (p35) |
| 182 | 205051_s_at | NM_000222 | KIT | v-kit Hardy-Zuckerman 4 feline |
| | | | | sarcoma viral oncogene homolog |
| 183 | 205142_x_at | NM_000033 | ABCD1 | ATP-binding cassette, sub-family D |
| | | | | (ALD), mem 1 |
| 184 | 205169_at | NM_005057 | RBBP5 | retinoblastoma-binding protein 5 |
| 185 | 205373_at | NM_004389 | CTNNA2 | catenin alpha 2 |
| 186 | 205376_at | NM_003866 | INPP4B | inositol polyphosphate-4- |
| | | | | phosphatase, type II, 105 kD |
| 187 | 205405_at | NM_003966 | SEMA5A | sema domain, seven thrombospondin |
| | | | | repeats, transmembrane domain and |
| | | | | short cytoplasmic domain 5A |
| 188 | 205447_s_at | BE222201 | | mitogen-activated protein kinase |
| | | | | kinase kinase 12 |
| 189 | 205458_at | BG034972 | | melanocortin 1 receptor |
| 190 | 205566_at | NM_007011 | HS1-2 | putative transmembrane protein |
| 191 | 205591_at | NM_006334 | AMY | neuroblastoma (nerve tissue) protein |
| 192 | 205681_at | NM_004049 | BCL2A1 | BCL2-related protein A1 |
| 193 | 205690_s_at | NM_003910 | G10 | maternal G10 transcript |
| 194 | 205691_at | NM_004209 | SYNGR3 | synaptogyrin 3 |
| 195 | 205717_x_at | NM_002588 | PCDHGC3 | protocadherin gamma subfamily C, 3 |
| 196 | 205813_s_at | NM_000429 | MAT1A | methionine adenosyltransferase I, α |
| 197 | 205937_at | NM_006569 | CGR11 | cell growth regulatory with EF-hand |
| | | | | domain |
| 198 | 205945_at | NM_000565 | IL6R | interleukin 6 receptor |
| 199 | 205996_s_at | NM_013411 | AK2 B | adenylate kinase 2 |
| 200 | 206128_at | AI264306 | | adrenergic, alpha-2C-, receptor |
| 201 | 206307_s_at | NM_004472 | FOXD1 | forkhead box D1 |
| 202 | 206332_s_at | NM_005531 | IFI16 | interferon, gamma-inducible 16 |
| 203 | 206397_x_at | NM_001492 | GDF1 | growth differentiation factor 1 |
| 204 | 206441_s_at | NM_017828 | FLJ20452 | hypothetical protein FLJ20452 |
| 205 | 206462_s_at | NM_002530 | NTRK3 | neurotrophic tyrosine kinase, |
| | | | | receptor, type 3 |
| 206 | 206503_x_at | NM_002675 | PML | promyelocytic leukemia |
| 207 | 206617_s_at | NM_002910 | RENBP | renin-binding protein |
| 208 | 206630_at | NM_000372 | TYR | tyrosinase |
| 209 | 206688_s_at | NM_006693 | CPSF4 | cleavage and polyadenylation specific |
| | | | | factor 4, 30 kD subunit |
| 210 | 206696_at | NM_000273 | OA1 | ocular albinism 1 |
| 211 | 206777_s_at | NM_000496 | CRYBB2 | crystallin, beta B2 |
| 212 | 206864_s_at | NM_003806 | HRK | harakiri, BCL2-interacting protein |
| 213 | 206976_s_at | NM_006644 | HSP105B | heat shock 105 kD |
| 214 | 207038_at | NM_004694 | SLC16A6 | solute carrier family 16 member 6 |
| 215 | 207060_at | NM_001427 | EN2 | engrailed homolog 2 |
| 216 | 207144_s_at | NM_004143 | CITED1 | Cbpp300-interacting transactivator, |
| | | | | with GluAsp-rich carboxy-terminal |
| | | | | domain, 1 |
| 217 | 207163_s_at | NM_005163 | AKT1 | v-akt murine thymoma viral oncogene |
| | | | | homolog 1 |
| 218 | 207183_at | NM_006143 | GPR19 | G protein-coupled receptor 19 |
| 219 | 207592_s_at | NM_001194 | HCN2 | hyperpolarization activated cyclic |
| | | | | nucleotide-gated potassium channel 2 |
| 220 | 207614_s_at | NM_003592 | CUL1 | cullin 1 |
| 221 | 207622_s_at | NM_005692 | ABCF2 | ATP-binding cassette, sub-fam F |
| | | | | mem 2 |
| 222 | 207828_s_at | NM_005196 | CENPF | centromere protein F |
| 223 | 208002_s_at | NM_007274 | HBACH | cytosolic acyl coenzyme A thioester |
| | | | | hydrolase |
| 224 | 208089_s_at | NM_030794 | FLJ21007 | hypothetical protein FLJ21007 |
| 225 | 208308_s_at | NM_000175 | GPI | glucose phosphate isomerase |
| 226 | 208540_x_at | NM_021039 | S100A14 | S100 calcium-binding protein A14 |
| 227 | 208644_at | M32721 | | poly(ADP-ribose) polymerase |
| 228 | 208657_s_at | AF142408 | | cell division control protein septin D1 |
| 229 | 208677_s_at | AL550657 | | Basigin |
| 230 | 208696_at | AF275798 | | PNAS-102 |
| 231 | 208710_s_at | AI424923 | | adaptor-related protein complex 3, |
| | | | | delta 1 subunit |
| 232 | 208723_at | BC000350 | | ubiquitin specific protease 11 |
| 233 | 208744_x_at | BG403660 | | heat shock 105 kD |
| 234 | 208837_at | BC000027 | | integral type I protein |
| 235 | 208916_at | AF105230 | SLC1A5 | neutral amino acid transporter |
| 236 | 208928_at | AF258341 | | NADPH-cytochrome P450 reductase |
| 237 | 208956_x_at | U62891 | DUT | deoxyuridine triphosphatase |
| 238 | 208974_x_at | BC003572 | | karyopherin (importin) beta 1 |
| 239 | 208975_s_at | L38951 | | importin beta subunit |
| 240 | 209015_s_at | BC002446 | | MRJ gene for a member of DNAJ fam |
| 241 | 209036_s_at | BC001917 | | malate dehydrogenase 2, NAD |
| 242 | 209053_s_at | AF083389 | | Wolf-Hirschhorn syn candidate 1 |
| 243 | 209072_at | M13577 | MBP | myelin basic protein |
| 244 | 209079_x_at | AF152318 | PCDH-gamma- | protocadherin gamma A1 |
| | | | A1 | |
| 245 | 209081_s_at | NM_030582 | COL18A1 | collagen, type XVIII, alpha 1 |
| 246 | 209123_at | BC000576 | | quinoid dihydropteridine reductase |
| 247 | 209132_s_at | BE313890 | | hypothetical protein FLJ20452 |
| 248 | 209172_s_at | U30872 | | mitosin |
| 249 | 209197_at | AA626780 | | KIAA0080 protein |
| 250 | 209198_s_at | BC004291 | | Similar to synaptotagmin 11 |
| 251 | 209247_s_at | BC001661 | | ATP-binding cassette, sub-fam F |
| | | | | mem 2 |
| 252 | 209254_at | AF277177 | | KIAA0265 protein |
| 253 | 209255_at | AF277177 | | KIAA0265 protein |
| 254 | 209256_s_at | AF277177 | | PNAS-119 |
| 255 | 209283_at | AF007162 | | unknown mRNA |
| 256 | 209345_s_at | AL561930 | | phosphatidylinositol 4-kinase type II |
| 257 | 209407_s_at | AF068892 | | Dukes type B colon adenocarcinoma |
| | | | | truncated suppressin |
| 258 | 209515_s_at | U38654 | | Rab27a |
| 259 | 209773_s_at | BC001886 | | ribonucleotide reductase M2 polypep |
| 260 | 209825_s_at | BC002906 | | Sim to uridine monophosphate kinase |
| 261 | 209827_s_at | NM_004513 | IL16 | interleukin 16 |
| 262 | 209828_s_at | M90391 | | putative IL-16 protein precursor |
| 263 | 209848_s_at | U01874 | | me20m |
| 264 | 209875_s_at | M83248 | | nephropontin |
| 265 | 209932_s_at | U90223 | | deoxyuridine triphosphate |
| | | | | nucleotidohydrolase precursor |
| 266 | 210052_s_at | AF098158 | | restricted expressed proliferation |
| | | | | associated protein 100 |
| 267 | 210073_at | L32867 | | alpha 2,8-sialyltransferase |
| 268 | 210111_s_at | AF277175 | | PNAS-138 |
| 269 | 210127_at | BC002510 | | small GTPase RAB6B |
| 270 | 210854_x_at | U17986 | | GABAnoradrenaline transporter |
| 271 | 210926_at | AY014272 | FKSG30 | FKSG30 |
| 272 | 210948_s_at | AF294627 | LEF1 | lymphoid enhancer factor 1 isoform |
| 273 | 210951_x_at | AF125393 | | Rab27 isoform |
| 274 | 211013_x_at | AF230411 | | tripartite motif protein TRIM19 lambda |
| 275 | 211052_s_at | BC006364 | | clone MGC: 12705, |
| 276 | 211066_x_at | BC006439 | | Similar to protocadherin gamma |
| | | | | subfamily A, 5 |
| 277 | 211373_s_at | U34349 | AD3LPAD5 | seven trans-membrane domain |
| 278 | 211564_s_at | BC003096 | | Sim to LIM domain protein |
| 279 | 211752_s_at | BC005954 | | clone MGC: 14592 |
| 280 | 211759_x_at | BC005969 | | clone MGC: 14625 |
| 281 | 211833_s_at | U19599 | | BAX delta |
| 282 | 212000_at | AB002363 | | KIAA0365 gene product |
| 283 | 212070_at | AL554008 | | G protein-coupled receptor 56 |
| 284 | 212081_x_at | AF129756 | MSH55 | MSH55 |
| 285 | 212119_at | BF348067 | | phosphatidylinositol glycan, class F |
| 286 | 212178_s_at | AK022555 | | FLJ12493 fis |
| 287 | 212193_s_at | BE881529 | | KIAA0731 protein |
| 288 | 212247_at | AW008531 | | KIAA0225 protein |
| 289 | 212285_s_at | AF016903 | | IMAGE: 3506210 |
| 290 | 212312_at | AL117381 | | clone RP5-857M17 on chrom 20 |
| 291 | 212338_at | AA621962 | | KIAA0727 protein |
| 292 | 212402_at | BE895685 | | KIAA0853 protein |
| 293 | 212472_at | BE965029 | | FLJ22463 fis |
| 294 | 212473_s_at | BE965029 | | FLJ22463 fis |
| 295 | 212512_s_at | AA551784 | | coactivator-associated arginine |
| | | | | methyltransferase-1 |
| 296 | 212520_s_at | AI684141 | | SWISNF related |
| 297 | 212552_at | BE617588 | | hippocalcin-like |
| 298 | 212715_s_at | AB020626 | | KIAA0819 protein |
| 299 | 212739_s_at | AL523860 | | non-metastatic cells 4 |
| 300 | 212744_at | AI813772 | | clone HQ0692 |
| 301 | 212745_s_at | AI813772 | | clone HQ0692 |
| 302 | 212793_at | BF513244 | | KIAA0381 protein |
| 303 | 212796_s_at | BF195608 | | KIAA1055 protein |
| 304 | 213002_at | BF347326 | MARCKS, 80K-L | myristoylated alanine-rich protein |
| | | | | kinase C substrate |
| 305 | 213007_at | BG478677 | | polymerase (DNA directed), gamma |
| 306 | 213008_at | BG478677 | | polymerase (DNA directed), gamma |
| 307 | 213096_at | T51252 | | KIAA0481 gene product |
| 308 | 213131_at | R38389 | | olfactomedin related ER localized |
| 309 | 213169_at | BG109855 | | clone TUA8 Cri-du-chat region |
| 310 | 213215_at | AI910895 | | clone EUROIMAGE 42138 |
| 311 | 213217_at | AU149572 | | adenylate cyclase 2(brain) |
| 312 | 213241_at | AF035307 | | clone 23785 |
| 313 | 213274_s_at | BE875786 | | cathepsin B |
| 314 | 213275_x_at | BE875786 | | cathepsin B |
| 315 | 213330_s_at | BE886580 | | stress-induced-phosphoprotein 1 |
| 316 | 213333_at | AL520774 | | malate dehydrogenase 2, NAD |
| 317 | 213338_at | BF062629 | | DKFZP586E1621 protein |
| 318 | 213392_at | AW070229 | | G protein-coupled receptor, fam C, |
| | | | | group 5, mem B |
| 319 | 213474_at | AI890903 | | ESTs |
| 320 | 213496_at | AW592563 | | KIAA0455 gene product |
| 321 | 213573_at | AA861608 | | karyopherin (importin) beta 1 |
| 322 | 213587_s_at | AI884867 | | ribosomal protein L26 |
| 323 | 213638_at | AW054711 | | PAC 257A7 on chromosome 6p24 |
| 324 | 213670_x_at | AI768378 | | KIAA0618 gene product |
| 325 | 213720_s_at | AI831675 | | SWISNF related, matrix associated, |
| | | | | actin dependent regulator of |
| | | | | chromatin, subfam a, member 4 |
| 326 | 213746_s_at | AW051856 | | filamin A, alpha |
| 327 | 213836_s_at | AW052084 | | KIAA1001 protein |
| 328 | 213895_at | BF445047 | | epithelial membrane protein 1 |
| 329 | 213960_at | T87225 | | CLONE = IMAGE: 22392 |
| 330 | 214023_x_at | AL533838 | | tubulin, beta polypeptide |
| 331 | 214068_at | AF070610 | | clone 24505 |
| 332 | 214104_at | AI703188 | | G-protein coupled receptor |
| 333 | 214201_x_at | AA742237 | | HLA-B associated transcript-2 |
| 334 | 214581_x_at | BE568134 | | death receptor 6 |
| 335 | 214614_at | AI738662 | | homeo box HB9 |
| 336 | 214632_at | AA295257 | | neuropilin 2 |
| 337 | 214656_x_at | BE790157 | | myosin IB |
| 338 | 214687_x_at | AK026577 | | FLJ22924 fis |
| 339 | 214708_at | BG484314 | | syntrophin, beta 1 |
| 340 | 214710_s_at | BE407516 | | cyclin B1 |
| 341 | 214714_at | AK022360 | | FLJ12298 fis |
| 342 | 214717_at | AL137534 | | DKFZp434H1419 |
| 343 | 214752_x_at | AI625550 | | filamin A, alpha |
| 344 | 214778_at | AB011541 | | MEGF8 |
| 345 | 214841_at | AF070524 | | clone 24453 |
| 346 | 214893_x_at | AI421964 | | hyperpolarization activated cyclic |
| | | | | nucleotide-gated potassium channel 2 |
| 347 | 214896_at | AL109671 | | EUROIMAGE 29222 |
| 348 | 215025_at | S76476 | | trkC {alternatively spliced} |
| 349 | 215115_x_at | AI613045 | | ets variant gene 6 (TEL oncogene) |
| 350 | 215126_at | AL109716 | | EUROIMAGE 208948 |
| 351 | 215155_at | J04178 | HEXA | abnormal β-hexosaminidase α chain |
| 352 | 215311_at | AL109696 | | EUROIMAGE 21920 |
| 353 | 215812_s_at | U41163 | SLC6A10 | creatine transporter |
| 354 | 215836_s_at | AK026188 | | FLJ22535 fis |
| 355 | 216194_s_at | AD001527 | | DNA from chrom 19-cosmid f24590 |
| | | | | containing CAPNS and POL2RI |
| 356 | 216973_s_at | S49765 | | homeo box B7 |
| 357 | 217033_x_at | S76475 | trkC | neurotrophic tyrosine kinase, |
| | | | | receptor, type 3 |
| 358 | 217104_at | AL109714 | | EUROIMAGE 327506 |
| 359 | 217226_s_at | M95929 | PHOX1 | Paired mesoderm homeo box 1 |
| 360 | 217297_s_at | AF143684 | MYO9b | unconventional myosin IXb |
| 361 | 217377_x_at | AF041811 | ETV6-NTRK3 | ETS related protein-growth factor |
| | | | fusion | receptor tyrosine kinase fusion |
| | | | | proteins |
| 362 | 217419_x_at | AK021586 | | FLJ11524 fis |
| 363 | 217624_at | AA464753 | | ESTs |
| 364 | 217799_x_at | NM_003344 | UBE2H | ubiquitin-conjugating enzyme E2H |
| 365 | 217827_s_at | NM_016630 | ACP33 | acid cluster protein 33 |
| 366 | 217867_x_at | NM_012105 | BACE2 | beta-site APP-cleaving enzyme 2 |
| 367 | 217874_at | NM_003849 | SUCLG1 | succinate-CoA ligase, GDP-forming, |
| | | | | alpha subunit |
| 368 | 217891_at | NM_022744 | FLJ13868 | hypothetical protein FLJ13868 |
| 369 | 218009_s_at | NM_003981 | PRC1 | protein regulator of cytokinesis 1 |
| 370 | 218030_at | NM_014030 | GIT1 | G protein-coupled receptor kinase- |
| | | | | interactor 1 |
| 371 | 218074_at | NM_016062 | LOC51647 | CGI-128 protein |
| 372 | 218143_s_at | NM_005697 | SCAMP2 | secretory carrier membrane protein 2 |
| 373 | 218151_x_at | NM_024531 | FLJ11856 | hypothetical protein FLJ11856 |
| 374 | 218152_at | NM_018200 | HMG20A | high-mobility group 20A |
| 375 | 218161_s_at | NM_017882 | FLJ20561 | hypothetical protein FLJ20561 |
| 376 | 218175_at | NM_025140 | FLJ22471 | hypothetical protein FLJ22471 |
| 377 | 218330_s_at | NM_018162 | FLJ10633 | hypothetical protein FLJ10633 |
| 378 | 218349_s_at | NM_017975 | FLJ10036 | hypothetical protein FLJ10036 |
| 379 | 218359_at | NM_024958 | FLJ23329 | hypothetical protein FLJ23329 |
| 380 | 218376_s_at | NM_022765 | FLJ11937 | hypothetical protein FLJ11937 |
| 381 | 218447_at | NM_020188 | DC13 | DC13 protein |
| 382 | 218542_at | NM_018131 | FLJ10540 | hypothetical protein FLJ10540 |
| 383 | 218564_at | BC002574 | FLJ10520 | hypothetical protein FLJ10520 |
| 384 | 218618_s_at | NM_022763 | FLJ23399 | hypothetical protein FLJ23399 |
| 385 | 218678_at | NM_024609 | FLJ21841 | hypothetical protein FLJ21841 |
| 386 | 218774_at | NM_014026 | HSPC015 | HSPC015 protein |
| 387 | 218786_at | NM_016575 | TU12B1-TY | TU12B1-TY protein |
| 388 | 218824_at | NM_018215 | FLJ10781 | hypothetical protein FLJ10781 |
| 389 | 218839_at | NM_012258 | HEY1 | hairyenhancer-of-split related with |
| | | | | YRPW motif 1 |
| 390 | 218856_at | NM_016629 | LOC51323 | hypothetical protein LOC51323 |
| 391 | 218888_s_at | NM_018092 | FLJ10430 | hypothetical protein FLJ10430 |
| 392 | 218931_at | NM_022449 | FLJ12538 | hypothetical protein FLJ12538 |
| 393 | 218952_at | NM_013271 | SAAS | granin-like neuroendocrine peptide |
| | | | | precursor |
| 394 | 218956_s_at | NM_015545 | KIAA0632 | KIAA0632 protein |
| 395 | 218980_at | NM_025135 | KIAA1695 | hypothetical protein FLJ22297 |
| 396 | 218996_at | NM_013342 | TFPT | TCF3 (E2A) fusion partner |
| 397 | 219011_at | NM_020904 | PLEKHA4 | pleckstrin homology domain- |
| | | | | containing, family A member 4 |
| 398 | 219039_at | NM_017789 | FLJ20369 | hypothetical protein FLJ20369 |
| 399 | 219040_at | NM_024535 | FLJ22021 | hypothetical protein FLJ22021 |
| 400 | 219041_s_at | NM_014374 | AP4 | zinc finger protein |
| 401 | 219051_x_at | NM_024042 | MGC2601 | hypothetical protein MGC2601 |
| 402 | 219066_at | NM_021823 | MDS018 | hypothetical protein MDS018 |
| 403 | 219066_at | NM_021823 | MDS018 | hypothetical protein MDS018 |
| 404 | 219148_at | NM_018492 | TOPK | PDZ-binding kinase; T-cell originated |
| | | | | protein kinase |
| 405 | 219152_at | NM_015720 | PODLX2 | endoglycan |
| 406 | 219219_at | NM_017854 | FLJ20512 | hypothetical protein FLJ20512 |
| 407 | 219361_s_at | NM_022767 | FLJ12484 | hypothetical protein FLJ12484 |
| 408 | 219372_at | NM_014055 | CDV-1 | CDV-1 protein |
| 409 | 219408_at | NM_019023 | FLJ10640 | hypothetical protein FLJ10640 |
| 410 | 219478_at | NM_021197 | WFDC1 | WAP four-disulfide core domain 1 |
| 411 | 219491_at | NM_024036 | MGC3103 | hypothetical protein MGC3103 |
| 412 | 219522_at | NM_014344 | FJX1 | putative secreted ligand homologous |
| | | | | to fjx1 |
| 413 | 219537_x_at | NM_016941 | DLL3 | Delta (Drosophila)-like 3 |
| 414 | 219555_s_at | NM_018455 | BM039 | uncharacterized bone marrow protein |
| | | | | BM039 |
| 415 | 219578_s_at | NM_030594 | FLJ13203 | hypothetical protein FLJ13203 |
| 416 | 219634_at | NM_018413 | C4ST | chondroitin 4-sulfotransferase |
| 417 | 219637_at | NM_025139 | FLJ12584 | hypothetical protein FLJ12584 |
| 418 | 219703_at | NM_018365 | FLJ11222 | hypothetical protein FLJ11222 |
| 419 | 219742_at | NM_030567 | MGC10772 | hypothetical protein MGC10772 |
| 420 | 219895_at | NM_017938 | FLJ20716 | hypothetical protein FLJ20716 |
| 421 | 219933_at | NM_016066 | LOC51022 | CGI-133 protein |
| 422 | 220116_at | NM_021614 | KCNN2 | potassium intermediatesmall |
| | | | | conductance calcium-activated |
| | | | | channel, subfamily N, member 2 |
| 423 | 220155_s_at | NM_023924 | FLJ13441 | hypothetical protein FLJ13441 |
| 424 | 220178_at | NM_021731 | PP3501 | hypothetical protein PP3501 |
| 425 | 220454_s_at | NM_020796 | SEMA6A | sema domain, transmembrane |
| | | | | domain and cytoplasmic domain, 6A |
| 426 | 220864_s_at | NM_015965 | LOC51079 | CGI-39 protein; cell death-regulatory |
| | | | | protein GRIM19 |
| 427 | 220948_s_at | NM_000701 | ATP1A1 | ATPase, Na+K+ transporting, alpha 1 |
| | | | | polypeptide |
| 428 | 220973_s_at | NM_030974 | | hypothetical protein DKFZp434N1923 |
| 429 | 220974_x_at | NM_030971 | BA108L7.2 | similar to rat tricarboxylate carrier-like |
| 430 | 220980_s_at | NM_031284 | | hypothetical protein DKFZp434B195 |
| 431 | 221059_s_at | NM_021615 | CHST6 | carbohydrate sulfotransferase 6 |
| 432 | 221483_s_at | AF084555 | ARPP-19 | okadaic acid-inducible and cAMP- |
| | | | | regulated phosphoprotein 19 |
| 433 | 221484_at | NM_004776 | | UDP-Gal:betaGlcNAc beta 1,4- |
| | | | | galactosyltransferase, polypeptide 5 |
| 434 | 221538_s_at | AL136663 | | DKFZp564A176 |
| 435 | 221558_s_at | AF288571 | LEF1 | lymphoid enhancer factor-1 |
| 436 | 221577_x_at | AF003934 | | prostate differentiation factor |
| 437 | 221641_s_at | AF241787 | | CGI16-iso |
| 438 | 221688_s_at | AL136913 | | DKFZp586L0118 |
| 439 | 221710_x_at | BC006241 | | hypothetical protein FLJ10647 |
| 440 | 221732_at | AK026161 | | FLJ22508 fis |
| 441 | 221759_at | AL583123 | | CLONE = CS0DL009YN09 |
| 442 | 221797_at | AY007126 | | clone CDABP0028 |
| 443 | 221799_at | AB037823 | | KIAA1402 protein, |
| 444 | 221815_at | BE671816 | | ESTs |
| 445 | 221882_s_at | AI636233 | | five-span transmembrane protein M83 |
| 446 | 221902_at | AL567940 | | CLONE = CS0DF036YK19 |
| 447 | 221909_at | BF984207 | | ESTs |
| 448 | 221962_s_at | AI829920 | | ubiquitin-conjugating enzyme E2H |
| 449 | 222116_s_at | AL157485 | | DKFZp762O207 |
| 450 | 222153_at | AK023133 | | FLJ13071 fis |
| 451 | 222155_s_at | AK021918 | | FLJ11856 fis |
| 452 | 222175_s_at | AK000003 | | FLJ00003 protein |
| 453 | 222196_at | AK000470 | | FLJ20463 fis |
| 454 | 222199_s_at | AK001289 | | FLJ10427 fis |
| 455 | 222206_s_at | AA781143 | | EUROIMAGE 2021883 |
| 456 | 222212_s_at | AK001105 | | FLJ10243 fis |
| 457 | 222231_s_at | AK025328 | | FLJ21675 fis |
| 458 | 222234_s_at | AK022644 | | FLJ12582 fis |
| 459 | 222240_s_at | AL137749 | | DKFZp434A0612 |
| 460 | 222294_s_at | AW971415 | | ESTs |
| 461 | 32811_at | X98507 | | myosin-I beta |
| 462 | 40560_at | U28049 | TXB2 | TBX2 |
| 463 | 44783_s_at | R61374 | | IMAGE-37665 |
| 464 | 46665_at | AI949392 | | IMAGE-2470926 |
| 465 | 55093_at | AA534198 | | IMAGE-993116 |
| 466 | 63825_at | AI557319 | | |
| 467 | 87100_at | AI832249 | | |
| 468 | 200017_at | NM_002954 | RPS27A | ribosomal protein S27a |
| 469 | 200606_at | NM_004415 | DSP | desmoplakin (DPI, DPII) |
| 470 | 200632_s_at | NM_006096 | NDRG1 | N-myc downstream regulated |
| 471 | 200636_s_at | NM_002840 | PTPRF | protein tyrosine phosphatase, |
| | | | | receptor type, F |
| 472 | 200795_at | NM_004684 | SPARCL1 | SPARC-like 1 |
| 473 | 200810_s_at | NM_001280 | CIRBP | cold inducible RNA-binding protein |
| 474 | 200897_s_at | NM_016081 | KIAA0992 | Palladin |
| 475 | 200953_s_at | NM_001759 | CCND2 | cyclin D2 |
| 476 | 200965_s_at | NM_006720 | ABLIM-s | actin binding LIM protein 1 transcript |
| | | | | variant |
| 477 | 201012_at | NM_000700 | ANXA1 | annexin A1 |
| 478 | 201041_s_at | NM_004417 | DUSP1 | dual specificity phosphatase 1 |
| 479 | 201125_s_at | NM_002213 | ITGB5 | integrin, beta 5 |
| 480 | 201200_at | NM_003851 | CREG | cellular repressor of E1A-stimulated |
| | | | | genes |
| 481 | 201286_at | Z48199 | syndecan 1 | syndecan-1 gene (exons 2-5) |
| 482 | 201328_at | AL575509 | | v-ets avian erythroblastosis virus E26 |
| | | | | oncogene homolog 2 |
| 483 | 201425_at | NM_000690 | ALDH2 | aldehyde dehydrogenase 2, |
| | | | | mitochondrial |
| 484 | 201427_s_at | NM_005410 | SEPP1 | selenoprotein P, plasma, 1 |
| 485 | 201432_at | NM_001752 | CAT | Catalase |
| 486 | 201540_at | NM_001449 | FHL1 | four and a half LIM domains 1 |
| 487 | 201667_at | NM_000165 | GJA1 | gap junction protein, alpha 1, 43 kD |
| 488 | 201681_s_at | AB011155 | KIAA0583 | KIAA0583 |
| 489 | 201798_s_at | NM_013451 | FER1L3 | fer-1 (C. elegans)-like 3 (myoferlin) |
| 490 | 201820_at | NM_000424 | KRT5 | keratin 5 |
| 491 | 201829_at | AW263232 | NET1 | neuroepithelial cell transf gene 1 |
| 492 | 201830_s_at | NM_005863 | NET1 | neuroepithelial cell transf gene 1 |
| 493 | 201839_s_at | NM_002354 | TACSTD1 | tumor-associated calcium signal |
| | | | | transducer 1 |
| 494 | 201842_s_at | AI826799 | | EGF-containing fibulin-like |
| | | | | extracellular matrix protein 1 |
| 495 | 201843_s_at | NM_004105 | EFEMP1 | EGF-containing fibulin-like |
| | | | | extracellular matrix protein 1 |
| | | | | transcript variant 1 |
| 496 | 201983_s_at | AW157070 | | epidermal growth factor receptor |
| 497 | 201984_s_at | NM_005228 | EGFR | epidermal growth factor receptor |
| 498 | 202054_s_at | NM_000382 | ALDH3A2 | aldehyde dehydrogenase 3 family, |
| | | | | member A2 |
| 499 | 202085_at | NM_004817 | TJP2 | tight junction protein 2 |
| 500 | 202193_s_at | NM_001144 | AMFR | autocrine motility factor receptor |
| 501 | 202196_s_at | NM_013253 | DKK3 | dickkopf (Xenopus laevis) homolog 3 |
| 502 | 202242_at | NM_004615 | TM4SF2 | transmembrane 4 superfamily mem 2 |
| 503 | 202267_at | NM_005562 | LAMC2 | laminin, gamma 2, transcript variant 1 |
| 504 | 202286_s_at | J04152 | GA733-1 | gastrointestinal tumor-assoc antigen |
| 505 | 202289_s_at | NM_006997 | TACC2 | transforming, acidic coiled-coil |
| | | | | containing protein 2 |
| 506 | 202350_s_at | NM_002380 | MATN2 | matrilin 2 precursor, transcript var 1 |
| 507 | 202387_at | NM_004323 | BAG1 | BCL2-associated athanogene |
| 508 | 202489_s_at | BC005238 | | FXYD domain-containing ion |
| | | | | transport regulator 3 |
| 509 | 202525_at | NM_002773 | PRSS8 | protease, serine, 8 (prostasin) |
| 510 | 202552_s_at | NM_016441 | CRIM1 | cysteine-rich motor neuron 1 |
| 511 | 202565_s_at | NM_003174 | SVIL | supervillin transcript variant 1 |
| 512 | 202575_at | NM_001878 | CRABP2 | cellular retinoic acid-binding protein 2 |
| 513 | 202597_at | AU144284 | | interferon regulatory factor 6 |
| 514 | 202668_at | BF001670 | | ephrin-B2 |
| 515 | 202712_s_at | NM_020990 | CKMT1 | creatine kinase, mitochondrial 1 |
| | | | | nuclear gene mitochondrial protein |
| 516 | 202746_at | AL021786 | | PAC 696H22 on chrom Xq21.1-21.2 |
| 517 | 202826_at | NM_003710 | SPINT1 | serine protease inhibitor, Kunitz t 1 |
| 518 | 202890_at | T62571 | | microtubule-associated protein 7 |
| 519 | 202936_s_at | NM_000346 | SOX9 | SRY-box 9 |
| 520 | 202994_s_at | Z95331 | | clone CTA-941F9 on chrom 22q13 |
| 521 | 203037_s_at | NM_014751 | KIAA0429 | KIAA0429 gene product |
| 522 | 203074_at | NM_001630 | ANXA8 | annexin A8 |
| 523 | 203081_at | NM_020248 | LOC56998 | beta-catenin-interacting protein ICAT |
| 524 | 203126_at | NM_014214 | IMPA2 | inositol(myo)-1(or 4)- |
| | | | | monophosphatase 2 |
| 525 | 203178_at | NM_001482 | GATM | glycine amidinotransferase |
| 526 | 203240_at | NM_003890 | FC(γ)BP | IgG Fc binding protein |
| 527 | 203327_at | N22903 | | insulin-degrading enzyme |
| 528 | 203355_s_at | NM_015310 | KIAA0942 | KIAA0942 protein |
| 529 | 203407_at | NM_002705 | PPL | Periplakin |
| 530 | 203408_s_at | NM_002971 | SATB1 | special AT-rich sequence binding |
| | | | | protein 1 |
| 531 | 203430_at | NM_014320 | SOUL | putative heme-binding protein |
| 532 | 203453_at | NM_001038 | SCNN1A | Na channel, nonvoltage-gated 1 α |
| 533 | 203485_at | NM_021136 | RTN1 | reticulon 1 |
| 534 | 203549_s_at | NM_000237 | LPL | lipoprotein lipase |
| 535 | 203571_s_at | NM_006829 | APM2 | adipose specific 2 |
| 536 | 203585_at | NM_007150 | ZNF185 | zinc finger protein 185 (LIM domain) |
| 537 | 203636_at | BE967532 | MID1 | midline 1 (OpitzBBB syndrome) |
| 538 | 203637_s_at | NM_000381 | MID1 | |
| 539 | 203638_s_at | NM_022969 | FGFR2 | FGF receptor 2 transcript var 2 |
| 540 | 203678_at | NM_014967 | KIAA1018 | KIAA1018 protein |
| 541 | 203687_at | NM_002996 | SCYD1 | small inducible cytokine subfam D |
| | | | | (Cys-X3-Cys) mem 1 |
| 542 | 203726_s_at | NM_000227 | LAMA3 | laminin, alpha 3 |
| 543 | 203786_s_at | NM_003287 | TPD52L1 | tumor protein D52-like 1 |
| 544 | 203797_at | AF039555 | VSNL1 | visinin-like protein 1 |
| 545 | 203799_at | NM_014880 | KIAA0022 | KIAA0022 gene product |
| 546 | 203812_at | AB011538 | | MEGF5 |
| 547 | 203881_s_at | NM_004010 | | dystrophin transcript variant Dp427p2 |
| 548 | 203910_at | NM_004815 | PARG1 | PTPL1-associated RhoGAP 1 |
| 549 | 203917_at | NM_001338 | CXADR | coxsackie virus and adenovirus |
| | | | | receptor |
| 550 | 203961_at | AL157398 | NEBL | nebulette protein |
| 551 | 203962_s_at | NM_006393 | NEBL | |
| 552 | 203963_at | NM_001218 | CA12 | carbonic anhydrase XII |
| 553 | 203992_s_at | AF000992 | UTX | ubiquitous TPR motif, X isoform |
| | | | | alternative transcript 1 |
| 554 | 203997_at | NM_002829 | PTPN3 | protein tyrosine phosphatase, non- |
| | | | | receptor type 3 |
| 555 | 204005_s_at | NM_002583 | PAWR | PRKC, apoptosis, WT1, regulator |
| 556 | 204019_s_at | NM_015677 | | hypothetical protein DKFZP586F1318 |
| 557 | 204036_at | AW269335 | | endothelial differentiation, |
| | | | | lysophosphatidic acid G-protein- |
| | | | | coupled receptor, 2 |
| 558 | 204037_at | AW269335 | | endothelial differentiation, |
| | | | | lysophosphatidic acid G-protein- |
| | | | | coupled receptor, 2 |
| 559 | 204042_at | AB020707 | | KIAA0900 protein |
| 560 | 204058_at | AL049699 | | clone 747H23 on chrom 6q13-15 |
| 561 | 204059_s_at | NM_002395 | ME1 | malic enzyme 1, NADP(+)-dependent, |
| | | | | cytosolic |
| 562 | 204072_s_at | NM_023037 | 13CDNA73 | putative gene product |
| 563 | 204112_s_at | NM_006895 | HNMT | histamine N-methyltransferase |
| 564 | 204135_at | NM_014890 | DOC1 | downregulated in ovarian cancer 1 |
| 565 | 204136_at | NM_000094 | COL7A1 | collagen, type VII, alpha 1 |
| 566 | 204151_x_at | NM_001353 | AKR1C1 | aldo-keto reductase family 1, mem C1 |
| 567 | 204154_at | NM_001801 | CDO1 | cysteine dioxygenase, type I |
| 568 | 204168_at | NM_002413 | MGST2 | microsomal glutathione S-transferase 2 |
| 569 | 204201_s_at | NM_006264 | PTPN13 | protein tyrosine phosphatase, non- |
| | | | | receptor type 13 |
| 570 | 204204_at | NM_001860 | SLC31A2 | solute carrier family 31 member 2 |
| 571 | 204224_s_at | NM_000161 | GCH1 | GTP cyclohydrolase 1 |
| 572 | 204254_s_at | NM_000376 | VDR | vitamin D receptor |
| 573 | 204345_at | NM_001856 | COL16A1 | collagen, type XVI, alpha 1 |
| 574 | 204351_at | NM_005980 | S100P | S100 calcium-binding protein P |
| 575 | 204359_at | NM_013231 | FLRT2 | fibronectin leucine rich |
| | | | | transmembrane protein 2 |
| 576 | 204363_at | NM_001993 | F3 | coagulation factor III |
| 577 | 204379_s_at | NM_000142 | FGFR3 | fibroblast growth factor receptor 3 |
| 578 | 204388_s_at | NM_000240 | MAOA | monoamine oxidase A |
| 579 | 204389_at | NM_000240 | MAOA | monoamine oxidase A |
| 580 | 204400_at | NM_005864 | EFS2 | signal transduction protein |
| 581 | 204421_s_at | M27968 | FGF | basic fibroblast growth factor |
| 582 | 204422_s_at | NM_002006 | FGF2 | fibroblast growth factor 2 (basic) |
| 583 | 204424_s_at | AL050152 | | neuronal specific transcription factor |
| | | | | DAT1 |
| 584 | 204455_at | NM_001723 | BPAG1 | bullous pemphigold antigen 1 |
| 585 | 204503_at | NM_001988 | EVPL | envoplakin |
| 586 | 204517_at | BE962749 | cyclophilin C | peptidylprolyl isomerase C |
| 587 | 204519_s_at | NM_015993 | LOC51090 | plasmolipin |
| 588 | 204537_s_at | NM_004961 | GABRE | gamma-aminobutyric acid A receptor, |
| | | | | epsilon transcript variant 1 |
| 589 | 204591_at | NM_006614 | CHL1 | cell adhesion molecule with homology |
| | | | | to L1CAM |
| 590 | 204600_at | NM_004443 | EPHB3 | EphB3 |
| 591 | 204671_s_at | BE677131 | | KIAA0957 protein |
| 592 | 204675_at | NM_001047 | SRD5A1 | steroid-5-α-reductase, α polypeptide 1 |
| 593 | 204718_at | NM_004445 | EPHB6 | EphB6 |
| 594 | 204719_at | NM_007168 | ABCA8 | ATP-binding cassette, sub-fam A |
| | | | | mem 8 |
| 595 | 204734_at | NM_002275 | KRT15 | keratin 15 |
| 596 | 204749_at | NM_004538 | NAP1L3 | nucleosome assembly protein 1-like 3 |
| 597 | 204753_s_at | AI810712 | | hepatic leukemia factor |
| 598 | 204754_at | AI810712 | | hepatic leukemia factor |
| 599 | 204755_x_at | M95585 | HLF | leukemia factor |
| 600 | 204765_at | NM_005435 | ARHGEF5 | Rho guanine nucleotide exchange |
| | | | | factor 5 |
| 601 | 204773_at | NM_004512 | IL11RA | interleukin 11 receptor, alpha |
| 602 | 204773_at | NM_004512 | IL11RA | |
| 603 | 204855_at | NM_002639 | SERPINB5 | serine (or cysteine) proteinase |
| | | | | inhibitor, clade B, member 5 |
| 604 | 204872_at | NM_007005 | BCE-1 | BCE-1 protein |
| 605 | 204937_s_at | NM_016325 | ZNF274 | zinc finger protein 274 |
| 606 | 204942_s_at | NM_000695 | ALDH3B2 | aldehyde dehydrogenase 3 fam mem |
| | | | | B2 |
| 607 | 204952_at | NM_014400 | C4.4A | GPI-anchored metastasis-associated |
| | | | | protein homolog |
| 608 | 204971_at | NM_005213 | CSTA | cystatin A (stefin A) |
| 609 | 204975_at | NM_001424 | EMP2 | epithelial membrane protein 2 |
| 610 | 204990_s_at | NM_000213 | ITGB4 | integrin, beta 4 |
| 611 | 205014_at | NM_005130 | HBP17 | heparin-binding growth factor binding |
| 612 | 205019_s_at | NM_004624 | VIPR1 | vasoactive intestinal pep receptor 1 |
| 613 | 205081_at | NM_001311 | CRIP1 | cysteine-rich protein 1 (intestinal) |
| 614 | 205109_s_at | NM_015320 | ARHGEF4 | Rho guanine nucleotide exchange |
| | | | | factor (GEF) 4 |
| 615 | 205128_x_at | NM_000962 | PTGS1 | prostaglandin-endoperoxide synthase 1 |
| 616 | 205185_at | NM_006846 | SPINK5 | serine protease inhibitor, Kazal t, 5 |
| 617 | 205200_at | NM_003278 | TNA | tetranectin |
| 618 | 205206_at | NM_000216 | KAL1 | Kallmann syndrome 1 sequence |
| 619 | 205236_x_at | NM_003102 | SOD3 | superoxide dismutase 3, extracellular |
| 620 | 205251_at | NM_022817 | PER2 | period homolog 2 transcript variant 1 |
| 621 | 205259_at | NM_000901 | NR3C2 | nuclear receptor subfamily 3, group |
| | | | | C, member 2 |
| 622 | 205286_at | U85658 | | transcription factor ERF-1 |
| 623 | 205349_at | NM_002068 | GNA15 | guanine nucleotide binding protein, α 15 |
| 624 | 205363_at | NM_003986 | BBOX1 | butyrobetaine (γ), 2-oxoglutarate |
| | | | | dioxygenase 1 |
| 625 | 205382_s_at | NM_001928 | DF | D component of complement (adipsin) |
| 626 | 205384_at | NM_005031 | FXYD1 | FXYD domain-containing ion |
| | | | | transport regulator 1 variant a |
| 627 | 205403_at | NM_004633 | IL1R2 | interleukin 1 receptor, type II |
| 628 | 205404_at | NM_005525 | HSD11B1 | hydroxysteroid dehydrogenase 1 |
| 629 | 205407_at | NM_021111 | RECK | reversion-inducing-cysteine-rich |
| | | | | protein with kazal motifs |
| 630 | 205440_s_at | NM_000909 | NPY1R | neuropeptide Y receptor Y1 |
| 631 | 205455_at | NM_002447 | MST1R | macrophage stimulating 1 receptor |
| 632 | 205464_at | NM_000336 | SCNN1B | Na channel, nonvoltage-gated 1, β |
| 633 | 205470_s_at | NM_006853 | KLK11 | kallikrein 11 |
| 634 | 205490_x_at | BF060667 | connexin 31 | gap junction protein, beta 3, 31 kD |
| 635 | 205498_at | NM_000163 | GHR | growth hormone receptor |
| 636 | 205559_s_at | NM_006200 | PCSK5 | proprotein convertase subtilisinkexin |
| | | | | type 5 |
| 637 | 205560_at | NM_006200 | PCSK5 | |
| 638 | 205569_at | NM_014398 | TSC403 | similar to lysosome-associated |
| | | | | membrane glycoprotein |
| 639 | 205613_at | NM_016524 | LOC51760 | BK protein |
| 640 | 205668_at | NM_002349 | LY75 | lymphocyte antigen 75 |
| 641 | 205672_at | NM_000380 | XPA | xeroderma pigmentosum, |
| | | | | complementation group A |
| 642 | 205709_s_at | NM_001263 | CDS1 | CDP-diacylglycerol synthase 1 |
| 643 | 205730_s_at | NM_014945 | KIAA0843 | KIAA0843 protein |
| 644 | 205765_at | NM_000777 | CYP3A5 | cyt P450, subfam IIIA, polypep 5 |
| 645 | 205807_s_at | NM_020127 | TUFT1 | tuftelin 1 |
| 646 | 205857_at | AI269290 | | solute carrier family 18, member 2 |
| 647 | 205883_at | NM_006006 | ZNF145 | zinc finger protein 145 |
| 648 | 205900_at | NM_006121 | KRT1 | keratin 1 |
| 649 | 205933_at | NM_015559 | KIAA0437 | KIAA0437 protein |
| 650 | 205977_s_at | NM_005232 | EPHA1 | EphA1 |
| 651 | 206032_at | AI797281 | | est:we86g02.x1 |
| 652 | 206033_s_at | NM_001941 | DSC3 | desmocollin 3 transcript variant Dsc3a |
| 653 | 206068_s_at | AI367275 | | acyl-Coenzyme A dehydrogenase, long chain |
| 654 | 206093_x_at | NM_007116 | TNXA | tenascin XA |
| 655 | 206122_at | NM_006942 | SOX20 | SRY-box 20 |
| 656 | 206149_at | NM_022097 | LOC63928 | hepatocellular carcinoma antigen |
| | | | | gene 520 |
| 657 | 206170_at | NM_000024 | ADRB2 | adrenergic, beta-2-, receptor, surface |
| 658 | 206192_at | L20815 | | S protein |
| 659 | 206201_s_at | NM_005924 | MEOX2 | mesenchyme homeo box 2 |
| 660 | 206276_at | NM_003695 | E48 | lymphocyte antigen 6 camp locus D |
| 661 | 206315_at | NM_004750 | CRLF1 | cytokine receptor-like factor 1 |
| 662 | 206363_at | NM_005360 | MAF | v-maf musculoaponeurotic |
| | | | | fibrosarcoma oncogene homolog |
| 663 | 206385_s_at | NM_020987 | ANK3 | ankyrin 3, node of Ranvier, tran var 1 |
| 664 | 206400_at | NM_002307 | LGALS7 | lectin, galactoside-binding, soluble, 7 |
| 665 | 206453_s_at | NM_016250 | NDRG2 | N-myc downstream-regulated gene 2 |
| 666 | 206481_s_at | NM_001290 | LDB2 | LIM domain binding 2 |
| 667 | 206482_at | NM_005975 | PTK6 | PTK6 protein tyrosine kinase 6 |
| 668 | 206515_at | NM_000896 | CYP4F3 | cyt P450, subfam IVF, polypeptide 3 |
| 669 | 206539_s_at | NM_023944 | CYP4F12 | cytochrome P450 isoform 4F12 |
| 670 | 206581_at | NM_001717 | BNC | basonuclin |
| 671 | 206637_at | NM_014879 | KIAA0001 | KIAA0001 gene product |
| 672 | 206655_s_at | NM_000407 | GP1BB | glycoprotein lb (platelet), β polypep |
| 673 | 206693_at | NM_000880 | IL7 | interleukin 7 |
| 674 | 206884_s_at | NM_003843 | SCEL | sciellin |
| 675 | 207002_s_at | NM_002656 | PLAGL1 | pleiomorphic adenoma gene-like 1 |
| 676 | 207023_x_at | NM_000421 | KRT10 | keratin 10 |
| 677 | 207076_s_at | NM_000050 | ASS | argininosuccinate synthetase |
| 678 | 207121_s_at | NM_002748 | MAPK6 | mitogen-activated protein kinase 6 |
| 679 | 207655_s_at | NM_013314 | SLP65 | B cell linker protein |
| 680 | 207720_at | NM_000427 | LOR | loricrin |
| 681 | 207761_s_at | NM_014033 | | DKFZP586A0522 protein |
| 682 | 207843_x_at | NM_001914 | CYB5 | cytochrome b-5 |
| 683 | 207908_at | NM_000423 | KRT2A | keratin 2A |
| 684 | 207943_x_at | NM_006718 | PLAGL1 | pleiomorphic adenoma gene-like |
| | | | | transcript variant 2 |
| 685 | 207955_at | NM_006664 | SCYA27 | small inducible cytokine subfamily A |
| | | | | (Cys—Cys), member 27 |
| 686 | 207996_s_at | NM_004338 | C18ORF1 | chrom 18 open reading frame 1 |
| 687 | 208096_s_at | NM_030520 | | hypothetical protein DKFZp564B052 |
| 688 | 208146_s_at | NM_031311 | LOC54504 | serine carboxypeptidase vitellogenic- |
| | | | | like |
| 689 | 208161_s_at | NM_020037 | ABCC3 | ATP-binding cassette sub-fam C mem 3 |
| 690 | 208190_s_at | NM_015925 | LISCH7 | liver-specific bHLH-Zip transcription |
| | | | | factor |
| 691 | 208228_s_at | M87771 | K-sam-III | secreted FGF receptor |
| 692 | 208609_s_at | NM_019105 | TNXB | tenascin XB |
| 693 | 208614_s_at | M62994 | | thyrold autoantigen |
| 694 | 208651_x_at | M58664 | | CD24 signal transducer |
| 695 | 208690_s_at | BC000915 | | Similar to LIM protein, |
| 696 | 208798_x_at | AF204231 | GM88 | 88-kDa Golgi protein |
| 697 | 209047_at | AL518391 | | aquaporin 1 |
| 698 | 209159_s_at | AV724216 | | NDRG family, member 4 |
| 699 | 209160_at | AB018580 | hluPGFS | aldo-keto reductase family 1, mem C3 |
| 700 | 209211_at | AF132818 | CKLF | colon Kruppel-like factor |
| 701 | 209212_s_at | AB030824 | | transcription factor BTEB2 |
| 702 | 209289_at | AI700518 | | nuclear factor IB |
| 703 | 209290_s_at | BC001283 | | Similar to nuclear factor IB, |
| 704 | 209309_at | D90427 | | zinc-alpha2-glycoprotein |
| 705 | 209318_x_at | BG547855 | | pleiomorphic adenoma gene-like 1 |
| 706 | 209335_at | AI281593 | | decorin |
| 707 | 209348_s_at | AF055376 | c-maf | short form transcription factor C-MAF |
| 708 | 209351_at | BC002690 | | keratin 14 |
| 709 | 209357_at | AF109161 | MRG1 | p35srj |
| 710 | 209366_x_at | M22865 | | cytochrome b5 |
| 711 | 209368_at | AF233336 | EPHX2 | soluble epoxide hydrolase |
| 712 | 209386_at | AI346835 | | transmembrane 4 superfam mem 1 |
| 713 | 209392_at | L35594 | autotaxin | ectonucleotide |
| | | | | pyrophosphatasephosphodiesterase 2 |
| 714 | 209465_x_at | AL565812 | | pleiotrophin |
| 715 | 209493_at | AF338650 | AIPC | PDZ domain-containing protein AIPC |
| 716 | 209540_at | NM_000618 | somatomedin C | insulin-like growth factor 1 |
| 717 | 209550_at | U35139 | | NECDIN related protein |
| 718 | 209558_s_at | AB013384 | HIP1R | huntingtin interacting protein-1-related |
| 719 | 209590_at | AL157414 | | clone RP11-560A15 on chrom 20 |
| 720 | 209602_s_at | AI796169 | | GATA-binding protein 3 |
| 721 | 209603_at | AI796169 | | GATA-binding protein 3 |
| 722 | 209604_s_at | BC003070 | | GATA-binding protein 3, clone |
| | | | | MGC: 2346 |
| 723 | 209605_at | D87292 | rhodanese | thiosulfate sulfurtransferase |
| 724 | 209656_s_at | AL136550 | | DKFZp761J17121 |
| 725 | 209679_s_at | BC003379 | | hyp protein from clone 643, clone |
| | | | | MGC: 5115 |
| 726 | 209684_at | AL136924 | | DKFZp586G2120 |
| 727 | 209687_at | U19495 | hIRH | intercrine-alpha |
| 728 | 209691_s_at | BC003541 | FLJ10488 | hypothetical protein FLJ10488 |
| 729 | 209699_x_at | U05598 | | dihydrodlol dehydrogenase |
| 730 | 209732_at | NM_005127 | | Sim to C-type lectin, superfam mem 2 |
| 731 | 209763_at | AL049176 | | clone 141H5 on chrom Xq22.1-23 |
| 732 | 209771_x_at | AA761181 | | CD24 antigen |
| 733 | 209863_s_at | AF091627 | | CUSP |
| 734 | 209866_s_at | R50822 | | KIAA0768 protein |
| 735 | 209975_at | AF182276 | CYP2E1 | cytochrome P450-2E1 |
| 736 | 210059_s_at | BC000433 | | mitogen-activated protein kinase 13 |
| 737 | 210096_at | J02871 | | lung cytochrome P450 BI |
| 738 | 210128_s_at | U41070 | | P2 purinergic receptor |
| 739 | 210298_x_at | AF098518 | FHL1 | 4 and ½ LIM domains 1 protein isoform B |
| 740 | 210347_s_at | AF080216 | | C2H2-type zinc-finger protein |
| 741 | 210372_s_at | AF208012 | TPD52L2 | tumor protein D52-like 2 |
| 742 | 210397_at | U73945 | | beta-defensin-1 |
| 743 | 210619_s_at | AF173154 | HYAL1 | hyaluronoglucosaminidase 1 isof 2 |
| 744 | 210633_x_at | M19156 | KRT10 | acidic keratin-10 |
| 745 | 210715_s_at | AF027205 | kop | Kunitz-type protease inhibitor |
| 746 | 210880_s_at | AB001467 | Efs2 | |
| 747 | 210958_s_at | BC003646 | | clone MGC: 4693 |
| 748 | 211043_s_at | BC006332 | Lcb | clathrin, light polypeptide |
| 749 | 211105_s_at | U80918 | NF-ATcC | transcrition factor |
| 750 | 211382_s_at | AF220152 | TACC2 | transforming, acidic coiled-coil |
| | | | | containing protein 2 |
| 751 | 211458_s_at | AF180519 | | GABA-A receptor-associated |
| 752 | 211596_s_at | AB050468 | | membrane glycoprotein LIG-1 |
| 753 | 211597_s_at | AB059408 | SMAP31-12 | |
| 754 | 211653_x_at | M33376 | | pseudo-chlordecone reductase |
| 755 | 211712_s_at | BC005830 | | clone MGC: 1925 |
| 756 | 211734_s_at | BC005912 | | Fc fragment IgE, high affinity I, rec for α |
| | | | | polypep |
| 757 | 211841_s_at | U94510 | | lymphocyte associated receptor of |
| | | | | death 9, alternatively spliced |
| 758 | 211986_at | BG287862 | desmoyokin | AHNAK nucleoprotein |
| 759 | 212148_at | BF967998 | | FLJ12900 fis, |
| 760 | 212204_at | AF132733 | | DKFZP564G2022 protein |
| 761 | 212242_at | AL565074 | | tubulin, alpha 1 (testis specific) |
| 762 | 212327_at | AK027231 | | FLJ23578 fis, KIAA1102 protein |
| 763 | 212328_at | AK027231 | | FLJ23578 fis, KIAA1102 protein |
| 764 | 212390_at | AB007923 | | KIAA0477 gene product |
| 765 | 212538_at | AL576253 | | KIAA1058 protein |
| 766 | 212543_at | U83115.1 | | non-lens β gamma-crystallin like |
| 767 | 212589_at | BG168858 | | oncogene TC21 |
| 768 | 212593_s_at | N92498 | | FLJ22071 fis, clone HEP11691 |
| 769 | 212724_at | BG054844 | | ras homolog gene family, member E |
| 770 | 212741_at | AA923354 | | monoamine oxidase A |
| 771 | 212823_s_at | AU147160 | | KIAA0599 protein |
| 772 | 212841_s_at | AI692180 | | PTPRF interacting protein, binding |
| | | | | protein 2 |
| 773 | 212850_s_at | AA584297 | | low density lipoprotein receptor-related |
| | | | | protein 4 |
| 774 | 212875_s_at | AP001745 | | chrom 21 open reading frame 25 |
| 775 | 212992_at | AI935123 | | ESTs |
| 776 | 213029_at | AL110126 | | DKFZp564H1916 |
| 777 | 213032_at | AL110126 | | DKFZp564H1916 |
| 778 | 213050_at | AA594937 | | KIAA0633 protein |
| 779 | 213068_at | AI146848 | | dermatopontin |
| 780 | 213071_at | AI146848 | | dermatopontin |
| 781 | 213106_at | AI769688 | | 23664 and 23905 mRNA sequence |
| 782 | 213110_s_at | AW052179 | | collagen, type IV, alpha 5 |
| 783 | 213122_at | AI096375 | | KIAA1750 protein, partial cds |
| 784 | 213135_at | U90902 | | clone 23612 |
| 785 | 213194_at | BF059159 | | Hs.301198 roundabout homolog |
| 786 | 213227_at | BE879873 | | progesterone membrane binding |
| 787 | 213280_at | AK000478 | | FLJ20471 fis |
| 788 | 213285_at | AV691491 | | DKFZp564D1462 |
| 789 | 213287_s_at | X14487 | | acidic (type I) cytokeratin 10 |
| 790 | 213353_at | BF693921 | | ATP-binding cassette, sub-family A, |
| | | | | member 5 |
| 791 | 213359_at | W74620 | | heterogenous nuclear |
| | | | | ribonucleoprotein D |
| 792 | 213369_at | AI825832 | | DKFZp434A132 |
| 793 | 213375_s_at | N80918 | | Novel gene mapping to chomo 13 |
| 794 | 213397_x_at | AI761728 | | DnaJ homolog, subfam C, mem 8 |
| 795 | 213451_x_at | BE044614 | | tenascin XB |
| 796 | 213456_at | AI927000 | | DKFZP564D206 |
| 797 | 213506_at | BE965369 | | proteinase activated receptor-2 |
| 798 | 213556_at | BE673445 | | chromosome 19, cosmid R28379 |
| 799 | 213618_at | AB011152 | | KIAA0580 |
| 800 | 213695_at | L48516 | PON3 | paraoxonase 3 |
| 801 | 213707_s_at | NM_005221 | DLX5 | distal-less homeo box 5 |
| 802 | 213725_x_at | AI693140 | | DKFZp586F071 |
| 803 | 213737_x_at | AI620911 | | |
| 804 | 213800_at | X04697 | | complement factor H 38-kDa N-term |
| 805 | 213817_at | AL049435 | | DKFZp586B0220 |
| 806 | 213820_s_at | T54159 | | hypothetical protein MGC10327 |
| 807 | 213844_at | NM_019102 | HOXA5 | homeo box A5 |
| 808 | 213848_at | AI655015 | | DKFZp586F2224 |
| 809 | 213891_s_at | AI927067 | | FLJ11918 fis |
| 810 | 213924_at | BF476502 | | hypothetical protein FLJ11585 |
| 811 | 213929_at | AL050204 | | DKFZp586F1223 |
| 812 | 213933_at | AW242315 | | DKFZp586M0723 |
| 813 | 213935_at | AF007132 | | clone 23551 |
| 814 | 213942_at | AL134303 | | DKFZp547K034_r1 |
| 815 | 213992_at | AI889941 | | collagen, type IV, alpha 6 |
| 816 | 213994_s_at | AI885290 | | spondin 1, extracellular matrix |
| 817 | 214058_at | M19720 | | L-myc protein |
| 818 | 214132_at | BG232034 | | ATP synthase, H+ transporting, |
| | | | | mitochondrial F1 complex, gamma |
| | | | | polypeptide 1 |
| 819 | 214164_x_at | BF752277 | | adaptor-related protein complex 1, |
| | | | | gamma 1 subunit |
| 820 | 214234_s_at | X90579 | | cyp related pseudogene |
| 821 | 214235_at | X90579 | | cyp related pseudogene |
| 822 | 214247_s_at | AU148057 | | regulated in glioma |
| 823 | 214598_at | AL049977 | | DKFZp564C122 |
| 824 | 214696_at | AF070569 | | clone 24659 |
| 825 | 214753_at | AW084068 | | BRCA2 region |
| 826 | 214823_at | AF033199 | | C2H2 zinc finger protein pseudogene |
| 827 | 215034_s_at | AI189753 | | FLJ13302 fis |
| 828 | 215062_at | AL390143 | | DKFZp547N074 |
| 829 | 215129_at | AJ000008 | | C2 domain containing PI3-kinase |
| 830 | 215239_x_at | AU132789 | | zinc finger protein 273 |
| 831 | 215243_s_at | AF099730 | GJB3 | connexin 31 |
| 832 | 215388_s_at | X56210 | FHR-1 | complement Factor H-related 1 |
| 833 | 215513_at | AF241534 | HYMAI | hydatidiform mole assoc & imprinted |
| 834 | 215516_at | AC005048 | | BAC clone CTB-15P3 fr 7q22-q31.2 |
| 835 | 215536_at | | | DMA, DMB, HLA-Z1, IPP2, LMP2, |
| | | | | TAP1, LMP7, TAP2, DOB, DQB2 AND |
| | | | | RING8, 9, 13 and 14 genes |
| 836 | 215659_at | AK025174 | | FLJ21521 |
| 837 | 215704_at | AL356504 | | clone RP1-14N1 chrom 1q21.1-21.3 |
| 838 | 215726_s_at | M22976 | | cytochrome b5 |
| 839 | 215867_x_at | AL050025 | | DKFZp564D066 |
| 840 | 216199_s_at | AL109942 | | clone RP3-473J16 chrom 6q25.3-26 |
| 841 | 216268_s_at | U77914 | | soluble protein Jagged |
| 842 | 216333_x_at | M25813 | | unidentified gene complementary to |
| | | | | P450c21 |
| 843 | 216379_x_at | AK000168 | | CD24 signal transducer |
| 844 | 216594_x_at | S68290 | | chlordecone reductase homolog |
| 845 | 216699_s_at | L10038 | | pre-pro-protein for kallikrein |
| 846 | 217087_at | AF005081 | xp32 | skin-specific protein |
| 847 | 217528_at | BF003134 | | ESTs |
| 848 | 217707_x_at | AI535683 | | ESTs |
| 849 | 217901_at | BF031829 | | desmoglein 2 |
| 850 | 217961_at | NM_017875 | FLJ20551 | hypothetical protein FLJ20551 |
| 851 | 218002_s_at | NM_004887 | SCYB14 | small inducible cytokine subfamily B |
| | | | | (Cys-X-Cys), member 14 |
| 852 | 218170_at | NM_016048 | LOC51015 | CGI-111 protein |
| 853 | 218180_s_at | NM_022772 | FLJ21935 | hypothetical protein FLJ21935 |
| 854 | 218186_at | NM_020387 | CATX-8 | CATX-8 protein |
| 855 | 218237_s_at | NM_030674 | ATA1 | amino acid transporter system A1 |
| 856 | 218326_s_at | NM_018490 | GPR48 | G protein-coupled receptor 48 |
| 857 | 218434_s_at | NM_023928 | | hypothetical protein FLJ12389 |
| 858 | 218451_at | NM_022842 | | hypothetical protein FLJ22969 |
| 859 | 218499_at | NM_016542 | LOC51765 | serinethreonine protein kinase MASK |
| 860 | 218546_at | NM_024709 | | hypothetical protein FLJ14146 |
| 861 | 218552_at | NM_018281 | | hypothetical protein FLJ10948 |
| 862 | 218603_at | NM_016217 | LOC51696 | hHDC for homolog of Dros headcase |
| 863 | 218644_at | NM_016445 | PLEK2 | pleckstrin 2 (mouse) homolog |
| 864 | 218651_s_at | NM_018357 | | hypothetical protein FLJ11196 |
| 865 | 218657_at | NM_016339 | LOC51195 | Link guanine nucleotide exchange |
| | | | | factor II |
| 866 | 218675_at | NM_020372 | LOC57100 | organic cation transporter |
| 867 | 218677_at | NM_020672 | LOC57402 | S100-type calcium binding protein A14 |
| 868 | 218692_at | NM_017786 | | hypothetical protein FLJ20366 |
| 869 | 218704_at | NM_017763 | | hypothetical protein FLJ20315 |
| 870 | 218718_at | NM_016205 | PDGFC | platelet derived growth factor C |
| 871 | 218736_s_at | NM_017734 | | hypothetical protein FLJ20271 |
| 872 | 218751_s_at | NM_018315 | | hypothetical protein FLJ11071 |
| 873 | 218764_at | NM_024064 | | hypothetical protein MGC5363 |
| 874 | 218792_s_at | NM_017688 | | hypothetical protein FLJ20150 |
| 875 | 218796_at | NM_017671 | | hypothetical protein FLJ20116 |
| 876 | 218804_at | NM_018043 | | hypothetical protein FLJ10261 |
| 877 | 218806_s_at | AF118887 | VAV-3 | VAV-3 protein |
| 878 | 218807_at | NM_006113 | VAV3 | vav 3 oncogene |
| 879 | 218816_at | NM_018214 | | hypothetical protein FLJ10775 |
| 880 | 218820_at | NM_020215 | | hypothetical protein DKFZp761F2014 |
| 881 | 218849_s_at | NM_006663 | RAI | RelA-associated inhibitor |
| 882 | 218854_at | NM_013352 | SART-2 | squamous cell carcinoma antigen recog |
| | | | | by T cell |
| 883 | 218901_at | NM_020353 | LOC57088 | phospholipid scramblase 4 |
| 884 | 218919_at | NM_024699 | | hypothetical protein FLJ14007 |
| 885 | 218963_s_at | NM_015515 | | DKFZP434G032 protein |
| 886 | 219010_at | NM_018265 | | hypothetical protein FLJ10901 |
| 887 | 219054_at | NM_024563 | | hypothetical protein FLJ14054 |
| 888 | 219064_at | NM_030569 | | hypothetical protein MGC10848 |
| 889 | 219073_s_at | NM_017784 | | hypothetical protein FLJ20363 |
| 890 | 219090_at | NM_020689 | NCKX3 | sodium calcium exchanger |
| 891 | 219093_at | NM_017933 | | hypothetical protein FLJ20701 |
| 892 | 219095_at | NM_005090 | PLA2G4B | phospholipase A2, group IVB |
| 893 | 219109_at | NM_024532 | | hypothetical protein FLJ22724 |
| 894 | 219115_s_at | NM_014432 | IL20RA | interleukin 20 receptor, alpha |
| 895 | 219229_at | NM_013272 | SLC21A11 | solute carrier family 21, member 11 |
| 896 | 219232_s_at | NM_022073 | | hypothetical protein FLJ21620 |
| 897 | 219263_at | NM_024539 | | hypothetical protein FLJ23516 |
| 898 | 219298_at | NM_024693 | | hypothetical protein FLJ20909 |
| 899 | 219313_at | NM_017577 | | hypothetical protein DKFZp434C0328 |
| 900 | 219368_at | NM_021963 | NAP1L2 | nucleosome assembly protein 1-like 2 |
| 901 | 219388_at | NM_024915 | | hypothetical protein FLJ13782 |
| 902 | 219395_at | NM_024939 | | hypothetical protein FLJ21918 |
| 903 | 219410_at | NM_018004 | | hypothetical protein FLJ10134 |
| 904 | 219411_at | NM_024712 | | hypothetical protein FLJ13824 |
| 905 | 219423_x_at | NM_003790 | TNFRSF12 | TNF receptor superfamily, member 12 |
| 906 | 219436_s_at | NM_016242 | LOC51705 | endomucin-2 |
| 907 | 219461_at | AJ236915 | | pak5 protein |
| 908 | 219476_at | NM_024115 | | hypothetical protein MGC4309 |
| 909 | 219489_s_at | NM_017821 | | hypothetical protein FLJ20435 |
| 910 | 219497_s_at | NM_022893 | BCL11A | B-cell CLLlymphoma 11A |
| 911 | 219518_s_at | NM_025165 | | hypothetical protein FLJ22637 |
| 912 | 219528_s_at | NM_022898 | BCL11B | B-cell lymphomaleukaemia 11B |
| 913 | 219532_at | NM_022726 | ELOVL4 | Stargardt disease 3 |
| 914 | 219597_s_at | NM_017434 | DUOX1 | dual oxidase 1 |
| 915 | 219689_at | NM_020163 | LOC56920 | semaphorin sem2 |
| 916 | 219729_at | NM_016307 | PRX2 | paired related homeobox protein |
| 917 | 219764_at | NM_007197 | FZD10 | frizzled (Drosophila) homolog 10 |
| 918 | 219806_s_at | NM_020179 | FN5 | FN5 protein |
| 919 | 219825_at | NM_019885 | P450RAI-2 | cyt P450 retinoid metabolizing |
| 920 | 219908_at | NM_014421 | DKK2 | dickkopf homolog 2 |
| 921 | 219936_s_at | NM_023915 | GPR87 | G protein-coupled receptor 87 |
| 922 | 219938_s_at | NM_024430 | PSTPIP2 | proline-serine-threonine phosphatase |
| | | | | interacting protein 2 |
| 923 | 219970_at | NM_017655 | | hypothetical protein FLJ20075 |
| 924 | 219976_at | NM_015888 | HOOK1 | hook1 protein |
| 925 | 219995_s_at | NM_024702 | | hypothetical protein FLJ13841 |
| 926 | 219998_at | NM_014181 | | HSPC159 protein |
| 927 | 220016_at | NM_024060 | | hypothetical protein MGC5395 |
| 928 | 220056_at | NM_021258 | IL22R | interleukin 22 receptor |
| 929 | 220066_at | NM_022162 | NOD2 | NOD2 protein |
| 930 | 220076_at | NM_019847 | ANKH | ankylosis, progressive homolog |
| 931 | 220161_s_at | NM_019114 | EHM2 | EHM2 gene |
| 932 | 220225_at | NM_016358 | IRX4 | iroquois homeobox protein 4 |
| 933 | 220230_s_at | NM_016229 | LOC51700 | cytochrome b5 reductase b5R.2 |
| 934 | 220262_s_at | NM_023932 | | hypothetical protein MGC2487 |
| 935 | 220266_s_at | NM_004235 | KLF4 | Kruppel-like factor 4 (gut) |
| 936 | 220289_s_at | NM_017977 | | hypothetical protein FLJ10040 |
| 937 | 220318_at | NM_017957 | FLJ20778 | epsin 3 |
| 938 | 220413_at | NM_014579 | ZIP2 | zinc transporter |
| 939 | 220414_at | NM_017422 | CLSP | calmodulin-like skin protein |
| 940 | 220428_at | NM_015717 | LANGERIN | Langerhans cell specific c-type lectin |
| 941 | 220432_s_at | NM_016593 | CYP39A1 | oxysterol 7alpha-hydroxylase |
| 942 | 220518_at | NM_024801 | | hypothetical protein FLJ21551 |
| 943 | 220625_s_at | AF115403 | | Ets transcription factor ESE-2b |
| 944 | 220723_s_at | NM_025087 | | hypothetical protein FLJ21511 |
| 945 | 220724_at | NM_025087 | | hypothetical protein FLJ21511 |
| 946 | 220911_s_at | NM_025081 | | KIAA1305 protein |
| 947 | 220945_x_at | NM_018050 | | hypothetical protein FLJ10298 |
| 948 | 221127_s_at | NM_006394 | RIG | regulated in glioma |
| 949 | 221215_s_at | NM_020639 | ANKRD3 | ankyrin repeat domain 3 |
| 950 | 221541_at | AL136861 | | DKFZp434B044 |
| 951 | 221667_s_at | AF133207 | | protein kinase H11 |
| 952 | 221747_at | AL046979 | | DKFZp586K0617 |
| 953 | 221748_s_at | AL046979 | | DKFZp586K0617 |
| 954 | 221760_at | BG287153 | | mannosidase, α, class 1A, member 1 |
| 955 | 221796_at | AA707199 | | Similar to hyp protein FLJ20093 |
| 956 | 221841_s_at | BF514079 | | Kruppel-like factor 4 (gut) |
| 957 | 221854_at | AI378979 | | ESTs |
| 958 | 221922_at | AW195581 | | KIAA0761 |
| 959 | 221950_at | AI478455 | | empty spiracles homolog 2 |
| 960 | 222043_at | AI982754 | | clusterin |
| 961 | 222102_at | NM_000847 | GSTA3 | glutathione S-transferase A3 |
| 962 | 222236_s_at | AK000253 | | FLJ20246 fis |
| 963 | 222256_s_at | AK000550 | | FLJ20543 fis |
| 964 | 222288_at | AI004009 | | ESTs |
| 965 | 222290_at | AA731709 | | ESTs |
| 966 | 222303_at | AV700891 | | ESTs |
| 967 | 266_s_at | L33930 | | CD24 signal transducer |
| 968 | 33322_i_at | X57348 | | clone 9112 |
| 969 | 33323_r_at | X57348 | | clone 9112 |
| 970 | 35666_at | U38276 | | semaphorin III family homolog |
| 971 | 38340_at | AB014555 | | KIAA0655 protein |
| 972 | 39248_at | N74607 | | za55a01.s1 |
| 973 | 40016_g_at | AB002301 | | KIAA0303 gene |
| 974 | 40093_at | X83425 | | LU gene Lutheran blood group |
| | | | | glycoprotein |
| 975 | 40472_at | AF007155 | | clone 23763 unknown mRNA |
| 976 | 57588_at | R62432 | | yg52e11.s1 |
| 977 | 60474_at | AA469071 | | ne17f11.s1 |
| 978 | 91826_at | AI219073 | | qg16e08.x1 |
| 979 | | | PBGD | |
| 980 | | | MART1 | |
| 981 | | | Me20m | |
| 982 | | | MAGE-3 | |
| 983 | | | | Me20m forward primer |
| 984 | | | | Me20m reverse primer |
| 985 | | | | Me20m probe |
| 986 | | | | PBGD forward primer |
| 987 | | | | PBGD reverse primer |
| 988 | | | | PBGD probe |
| 999 | | | | Tyrosinase |
| 1000 | | | | Tyrosinase Forward |
| 1001 | | | | Tyrosinase Reverse |
| 1002 | | | | Tyrosinase probe |
| 1003 | | | | MART1 Forward |
| 1004 | | | | MART1 Reverse |
| 1005 | | | | MART1 Probe |
| 1006 | | | | HMB45 Forward |
| 1007 | | | | gp100 Reverse |
| 1008 | | | | gp100 Probe |
| 1009 | | | | PLAB Forward |
| 1010 | | | | PLAB Reverse |
| 1011 | | | | PLAB Probe |

### REFERENCES CITED

W O 96/29430

2003/0232356

2002/0160382

2002/0155480

2002/0110820

2002/0098535

2003/0059431

2003/0049701

2002/0048784

2003/014283

2003/0013097

U.S. Pat. No. 6,500,919

U.S. Pat. No. 4,715,545

U.S. Pat. No. 5,210,015

U.S. Pat. No. 5,348,856

U.S. Pat. No. 5,411,876

U.S. Pat. No. 5,413,924

U.S. Pat. No. 5,487,972

U.S. Pat. No. 5,512,437

U.S. Pat. No. 5,512,444

U.S. Pat. No. 5,525,494

U.S. Pat. No. 5,550,044

U.S. Pat. No. 5,612,201

U.S. Pat. No. 5,759,783

U.S. Pat. No. 5,804,375

U.S. Pat. No. 5,844,075

U.S. Pat. No. 5,844,092

U.S. Pat. No. 5,872,225

U.S. Pat. No. 5,910,574

U.S. Pat. No. 5,969,124

U.S. Pat. No. 5,985,619

U.S. Pat. No. 5,994,102

U.S. Pat. No. 6,025,474

U.S. Pat. No. 6,057,105

U.S. Pat. No. 6,107,476

U.S. Pat. No. 6,153,388

U.S. Pat. No. 6,235,525

U.S. Pat. No. 6,291,430

U.S. Pat. No. 6,326,145

U.S. Pat. No. 6,338,947

U.S. Pat. No. 6,369,211

U.S. Pat. No. 6,403,341

U.S. Pat. No. 6,426,217

U.S. Pat. No. 6,465,181

U.S. Pat. No. 6,475,727

U.S. Pat. No. 6,500,638

U.S. Pat. No. 6,521,227

U.S. Pat. No. 6,527,560

U.S. Pat. No. 6,599,699

Abrahamsen et al. (2004) Quantification of melanoma mRNA markers in sentinel nodes: pre-clinical evaluation of a single-step real-time reverse transcriptase-polymerase chain reaction assay J Molec Diag 6:253-259

Abrahamsen et al. (2005) Pathologic assessment of melanoma sentinel nodes: a role for molecular analysis using quantitative real-time reverse transcription-PCR for MARTl and tyrosinase messenger RNA Clin Cancer Res 11:1425-1433

Adema et al. (1994) Molecular characterization of the melanocyte lineage specific antigen gplOO J Biol Chem 69:20126-20133

Ahmed et al. (2004) Cdc42-dependent nuclear translocation of non-receptor tyrosine kinase, ACK Biochem. Biophys. Res. Commun. 314:571-579

Aitken et al. (2004) Population screening for melanoma: current evidence and a community based randomized trial. In “Textbook of Melanoma” Ed. by J F Thompson, D L Morton and B B R Kroon. Martin and Dunitz

Akula et al. (2004) Raf promotes human herpesvirus-8 (HHV-8/KSHV) infection Oncogene 23:5227-5241

Alexa et al. (2004) Contribution of distinct structural elements to activation of calpain by Ca2+ ions J. Biol. Chem. 279:201 18-20126

Altznauer et al. (2004) Calpain-1 regulates Bax and subsequent Smac-dependent caspase-3 activation in neutrophil apoptosis J. Biol. Chem. 279:5947-5957

Antonescu et al. (2002) Molecular diagnosis of clear cell sarcoma: detection of EWS-ATFl and MITF-M transcripts and histopathological and ultrastructural analysis of 12 cases J. Mol. Diag. 4:44-52

Arozarena et al. (2004) Activation of H-Ras in the endoplasmic reticulum by the RasGRF family guanine nucleotide exchange factors MoI. Cell. Biol. 24:1516-1530

Bae et al. (2003) Gene expression patterns as potential molecular biomarkers for malignant transformation in human keratinocytes treated with MNNG, arsenic or a metal mixture Toxicol Sci 74:32-42

Balch et al. (2001) Final version of the American Joint Committee on cancer staging system for cutaneous melanoma J Clin Oncol 19:3635-3648

Baldi et al. (2003) Identification of genes down-regulated during melanoma progression: a cDNA array study Exp Dermatol 12:213-218

Barac et al. (2004) Direct interaction of p21 -activated kinase 4 with PDZ-RhoGEF, a G protein-linked Rho guanine exchange factor J. Biol. Chem. 279:6182-6189

Bendotti et al. (2004) Activated p38MAPK is a novel component of the intracellular-inclusions found in human amyotrophic lateral sclerosis and mutant SODl transgenic mice J. Neuropathol. Exp. Neurol. 63:113-119

Bittner et al. (2000) Molecular classification of cutaneous malignant melanoma by gene expression profiling Nature 406:536-540

Bostick et al. (1999) Prognostic significance of occult metastases detected by sentinel lymphadenectomy and reverse transcriptase-polymerase chain reaction in early-stage melanoma patients J. Clin. Oncol. 17:3238-3244

Brown et al. (2003) MIC-I Serum Level and Genotype: Associations with Progress and Prognosis of Colorectal Carcinoma Clin. Cancer Res. 9:2642-2650

Buckhaults et al. (2001) Secreted and cell surface genes expressed in benign and malignant colorectal tumors Cancer Res. 61:6996-7001

Burzio et al. (2002) Biochemical and cellular characteristics of the four splice variants of protein kinase CKl alpha from zebrafish (Danio rerio) J. Cell. Biochem. 86:805-814

Buchs et al. (2004) Normal p21 Ras/MAP kinase pathway expression and function in PBMC from patients with polycystic ovary disease Int. J. Mol. Med. 13:595-599

Cancer Facts and Figures 2003. American Cancer Society, 2003.

Care et al. (1996) HOXB7 constitutively activates basic fibroblast growth factor in melanomas MoI. Cell. Biol. 16:4842-4851

Carlson et al. (2003) Biomarkers in melanoma: staging, prognosis and detection of early metastasis Expert Rev MoI Diagn 3:89-116

Catala et al. (2002) Genetic control of caudal development Clin. Genet. 61:89-96

Chen et al. (2003) Molecular characterization of human ninein protein: two distinct subdomains required for centrosomal targeting and regulating signals in cell cycle Biochem. Biophys. Res. Commun. 308:975-983

Karan et al. (2003) Dysregulated expression of MIC-1/PDF in human prostate tumor cells Biochim. Biophys. Res. Commun. 305:598-604

Chen et al. (2004) MEKl,2 response element mediates angiotensin Il-stimulated plasminogen activator inhibitor-1 promoter activation Blood 103:2636-2644

Chen et al. (2004) Cdc42 and the actin-related protein/neural Wiskott-Aldrich syndrome protein network mediate cellular invasion by Cryptosporidium parvum Infect. Immun. 72:3011-3021

Cheung et al. (2001) Detection of microscopic disease: comparing histology, immunocytology, and RT-PCR of tyrosine hydroxylase, GAGE, and MAGE Med. Ped. Oncol. 36:210-212

Ching et al. (2003) Identification of an autoinhibitory domain of p21-activated protein kinase 5 J. Biol. Chem. 278:33621-33624

Clark et al. (2003) The secreted protein discovery initiative (SPDI), a large-scale effort to identify novel human secreted and transmembrane proteins: a bioinformatics assessment Genome Res. 13:2265-2270

Cochran et al. (2001) Current practice and future directions in pathology and laboratory evaluation of the sentinel node Ann Surg Oncol. 8(9 Suppl):13S-17S

Cochran et al. (2004) Optimized assessment of sentinel lymph nodes for metastatic melanoma: implications for regional surgery and overall treatment planning Ann. Surg. Oncol. 11:156S-161S

Cook et al (2003) The development of optimal pathological assessment of sentinel lymph nodes for melanoma J. Pathol. 200:314-319

Cronin et al. (2004) Measurement of gene expression in archival paraffin-embedded tissues: development and performance of a 92-gene reverse transcriptase-polymerase chain reaction assay Am J Pathol 164:35-42

Davids et al. (2003) Accurate molecular detection of melanoma nodal metastases: an assessment of multimarker assay specificity, sensitivity, and detection rate MoI. Pathol. 56:43-51

de Braud et al. (2003) Malignant melanoma Critical Review In Oncology/Hematology 47:35-63

Diego et al. (2003) Prognostic Significance of Reverse Transcriptase-Polymerase Chain Reaction-Negative Sentinel Nodes in Malignant Melanoma Ann. Surg. Oncol. 10:396-402

Ducy et al. (2000) The family of bone morphogenetic proteins Kidney int. 57:2207-2214

Dwivedi et al. (2004) Protein kinase A in postmortem brain of depressed suicide victims: altered expression of specific regulatory and catalytic subunits Biol. Psychiatry 55:234-243

Edgar et al. (2002) Cloning and tissue distribution of three murine alpha/beta hydrolase fold protein cDNAs Biophys. Biochim. Res. Comm. 292:617-625

Encinas et al. (2004) Tyrosine 981, a novel ret autophosphorylation site, binds c-Src to mediate neuronal survival J. Biol. Chem. 279:18262-18269

Esler et al. (2001) A portrait of Alzheimer secretases—new features and familiar faces Science 293:1449-1454

Evellin et al. (2002) Stimulation of phospholipase C-epsilon by the M3 muscarinic acetylcholine receptor mediated by cyclic AMP and the GTPase Rap2B J. Biol. Chem. 277:16805-16813

Fenner et al. (1998) MSGl (melanocyte-specific gene 1): mapping to chromosome XqI 3.1, genomic organization, and promoter analysis Genomics 51:401-407

Fogel et al. (2003) Ll adhesion molecule (CD171) in development and progression of human malignant melanoma Cancer Letters 189:237-247

Fujii et al. (2004) Am. J. Physiol. Cell Physiol. 287:C200-C208

Gangemi et al. (2004) Regulatory genes controlling cell fate choice in embryonic and adult neural stem cells J Neurochem 89:286-306

Giancotti et al. (1999) Integrin signaling Science 285:1028-1032

Golubovskaya et al. (2004) Cloning and characterization of the promoter region of human focal adhesion kinase gene: nuclear factor kappa B andp53 binding sites Biochim. Biophys. Acta 1678:111-125

Gosmanov et al. (2004) Impaired expression and insulin-stimulated phosphorylation of Akt-2 in muscle of obese patients with atypical diabetes Am. J. Physiol. Endocrinol. Metab. 287:E8-E15

Goydos et al. (2003) Patterns of recurrence in patients with melanoma and histologically negative but RT-PCR-positive sentinel lymph nodes J. Am. Coll. Surg. 196:196-204

Grimsley et al. (2004) Dockl80 and ELMOl proteins cooperate to promote evolutionarily conserved Rac-dependent cell migration J. Biol. Chem. 279:6087-6097

Gronholm et al. (2003) Merlin links to the cAMP neuronal signaling pathway by anchoring the RIbeta subunit of protein kinase A J. Biol. Chem. 278:41167-41172

Guan et al. (2003) ASC/TMS1, a caspase-1 activating adaptor, is down regulated by aberrant methylation in human melanoma Int J Cancer 107:202-208

Gustafson et al. (2004) Bcr-Abl Regulates Protein Kinase Ci (PKCt) Transcription via an Elkl Site in the PKCi Promoter J. Biol. Chem. 279:9400-9408

Gutzmer et al. (2002) Specificity of tyrosinase and HMB45 PCR in the detection of melanoma metastases in sentinel lymph node biopsies Histopathol 41:510-518

Haendeler et al. (2003) GITI mediates Src-dependent activation of phospholipase Cgamma by angiotensin II and epidermal growth factor J. Biol. Chem. 278:49936-49944

Hafher et al. (2003) Loss of EphB6 expression in metastatic melanoma Int. J. Oncol. 23:1553-1559

Haqq et al. (2005) The gene expression signature of melanoma progression Proc Natl Acad Sci USA 102:6092-6097

Haspel et al (2003) Front. Biosci. 8:S1210-S1225

Hendrix et al. (2002) Cancer Res 62:665-668

Henshall et al. (2003) Cancer Res. 63:4196-4203

Hilgard et al. (2004) Am. J. Physiol. Gastrointest. Liver Physiol. 287:G192-G201

Hipfel et al. (1998) RNA isolation from human skin tissues for colorimetric differential display J Biochem Biophys Method 37:131-135

Hipfel et al. (2000) Br. J. Cancer 82:1149-1157

Hiraga et al. (1997) Virchows Archiv. 431:45-51

Hisaoka et al. (2002) J. Pathol. 197:661-667

Hoek et al. (2004) Expression profiling reveals novel pathways in the transformation of melanocytes to melanomas Cancer Res 64:5270-5282

Hoon et al. (2001) J. Invest. Dermatol. 117:375-378

Hoon et al. (2004) Profiling epigenetic inactivation of tumor suppressor genes in tumor and plasma from cutaneous melanoma patients Oncogene 23:4014-4022

Izard et al. (2004) Nature 427:171-175

Jaffer et al. (2002) Int. J. Biochem. Cell Biol. 34:713-717

Kadaja et al. (2004) Oncogene 23:2523-2530

Kam et al. (2004) Neuropathol. Appl. Neurobiol. 30:225-232

Kamholz et al. (1996) Proc. Natl. Acad. Sci. USA 83:4962-4966

Kammula et al. (2004) Serial follow-up and prognostic significance of reverse transcriptase-polymerase chain reaction-staged sentinel lymph nodes from melanoma patients J Clin Oncol 22:3989-3996

Kaneda et al. (2002) Int. J. Cancer 100:57-62

Kikuchi et al. (2003) An N-terminal fragment of ProSAAS (a granin-like neuroendocrine peptide precursor) is associated with tau inclusions in Pick's disease Biochem. Biophys. Res. Commun. 308:646-654

Kirchhoff et al. (2004) J. Virol. 78:6864-6874

Kitano et al. (2003) Mol. Biol. Evol. 20:1281-1289

Kuo et al. (2003) Prediction of disease outcome in melanoma patients by molecular analysis of paraffin-embedded sentinel lymph nodes J Clin Oncol 21:3566-3572

Lee et al. (2004) J. Biol. Chem. 279:6834-6839

Lee et al. (2004) J. Neurosci. 24:2304-2312

Li et al. (1995) Proc. Natl. Acad. Sci. USA 92:121180-12184

Li et al. (2004) J. Biol. Chem. 279:15938-15945

Li et al. (2004) J. Biol. Chem. 279:20401-20410

Liu et al (2003) Cancer Res. 63:5034-5040

Liu et al. (1992) J. Biol. Chem. 267:15829-15835

Liu et al. (2003) Macrophage inhibitory cytokine 1 reduces cell adhesion and induces apoptosis in prostate cancer cells Cancer Res 63:5034-40

Luftner et al. (2003) Plasma levels of HER-2/neu, tumor type M2 pyruvate kinase and its tyrosine-phosphorylated metabolite in advanced breast cancer Anticancer Res. 23(2A):991-997

Luo et al. (2001) Human prostate cancer and benign prostatic hyperplasia: molecular dissection by gene expression profiling Cancer Res 61:4683-4688

Luthi et al. (2003) Exp. Hematol. 31:150-158

MacDougall et al. (2003) J. Clin. Endocrinol. Metab. 88:2194-2205

Majumder et al. (2004) Nat. Med. 10:594-601

Mandelcorn-Monson et al. (2003) J. Invest. Dermatol. 121:550-556

Marchetti et al. (2003) J. Cell. Biochem. 88:865-872

Maresh et al. (1994) DNA Cell. Biol. 13:87-95

Marlow et al. (2003) Biochem. Biophys. Res. Commun. 305:502-509

McGregor et al. (1999) Proc. Natl. Acad. Sci. USA 96:4540-4545

McMasters (2003) Molecular staging of melanoma: sensitivity, specificity and the search of clinical significance Ann Surg Oncol 10:336-337

Menez et al. (2004) Oncogene 23:2630-2639

Messina et al. (1999) Pathologic examination of the sentinel lymph node in malignant melanoma Am J Surg Pathol 23:686-690

Monaco (2004) Evidence regarding a role fro CDK5 dysregulation in Alzheimer's disease Curr Alzheimer Res 1:33-38

Morton et al. (1992) Technical details of intraoperative lymphatic mapping for early stage melanoma Arch Surg 127:392-399

Nakamura et al. (2003) Quantitative analysis of macrophage inhibitory cytokine-1 (MIC-I) gene expression in human prostatic tissues Br. J. Cancer 88:1101-1104

Nishihara et al. (2004) Cyclic AMP promotes cAMP-responsive element-binding protein-dependent induction of cellular inhibitor of apoptosis protein-2 and suppresses apoptosis of colon cancer cells through ERK1/2 and p38 MAPK J. Biol. Chem. 279:26176-26183

Nomura et al. (2004) Identification of a novel domain of Ras and Rapl that directs their differential subcellular localizations!. Biol. Chem. 279:22664-22673

Oda et al. (2003) CrkL directs ASAPl to peripheral focal adhesions J. Biol. Chem. 278:6456-6460

Okada et al. (2001) Genetic detection of lymph node micrometastases inpatients with gastric carcinoma by multiple-marker reverse transcriptase-polymerase chain reaction assay Cancer 92:2056-2064

Okami et al. (2001) Genetic detection for micrometastasis in lymph node of biliary tract carcinoma Clin. Cancer Res. 6:2326-2332

Ooka et al. (2000) Selection of mRNA markers for detection of lymph node micrometastases in breast cancer patients Oncol. Rep. 7:561-566

Oury et al. (2004) ATP augments von Willebrand factor-dependent shear-induced platelet aggregation through Ca2+-calmodulin and myosin light chain kinase activation J. Biol. Chem. 279:26266-26273

Palmieri et al. (2001) Detection of occult melanoma cells in paraffin-embedded histologically negative sentinel lymph nodes using a reverse transcriptase polymerase chain reaction assay J. Clin. Oncol. 19:1437-1443

Paralkar et al. (1998) Cloning and characterization of a novel member of the transforming growth factor-beta/bone morphogenetic protein family J. Biol. Chem. 273:13760-13767

Pastorino et al. (2004) BACE (beta-secretase) modulates the processing of APLP2 in vivo MoI. Cell. Neurosci. 25:642-649

Patel et al. (2003) Cadherin switching in ovarian cancer progression Int. J. Cancer 106:172-177

Pitsi et al. (2004) Presenilin 1 stabilizes the C-terminal fragment of the amyloid precursor protein independently of gamma-secretase activity J. Biol. Chem. 279:25333-25338

Masters et al. (1985) Amyloid plaque core protein in Alzheimer disease and Down syndrome Proc. Natl. Acad. Sci. U.S.A. 82:4245-4249

Polyak et al. (1997) A model for p53-induced apoptosis Nature 389:300-305

Pontow et al. (2004) Actin cytoskeletal reorganizations and coreceptor-mediated activation of rac during human immunodeficiency virus-induced cell fusion J. Virol. 78:7138-7147

Prichard et al. (2003) The role of molecular staging in malignant melanoma Eur. J. Surg. Oncol. 29:306-314

Qi et al. (2004) α-Chimaerin exists in a functional complex with the Cdk5 kinase in brain FEBS Lett. 561:177-180

Qi et al. (2004) Essential Role of p38 {gamma} in K-Ras Transformation Independent of Phosphorylation J. Biol. Chem. 279:22138-22144

Qin et al. (2004) p53-independent NOXA induction overcomes apoptotic resistance of malignant melanomas MoI Cancer Therap 3:895-902

Raas-Rothschild et al. (2002) A PEX6-defective peroxisomal biogenesis disorder with severe phenotype in an infant, versus mild phenotype resembling Usher syndrome in the affected parents Am. J. Hum. Genet. 70:1062-1068

Raich et al. (1986) Molecular cloning and complete primary sequence of human erythrocyte porphobilinogen deaminase Nucl. Acids Res. 14:5955-5968

Ray et al. (1997) AIMl, a novel non-lens member of the betagamma-crystallin superfamily, is associated with the control of tumorigenicity in human malignant melanoma Proc. Natl. Acad. Sci. USA 94:3229-3234

Reifenberger et al. (2004) Frequent alterations of Ras signaling pathway genes in sporadic malignant melanomas hit. J. Cancer 109:377-384

Reintgen et al. (2004) The staging of malignant melanoma and the Florida Melanoma Trial Ann. Surg. Oncol. 11(3 Suppl):186S-191S

Rliee et al. (2004) MEK is a key modulator for TLR5-induced interleukin-8 and MIP3alpha gene expression in non-transformed human colonic epithelial cells J. Biol. Chem. 279:25179-25188

Riccioni et al. (2002) The sentinel lymph node in melanoma: utilization of molecular biology (RT-PCR) to detect occult metastases Pathologica 94:190-195

Rimboldi et al. (2003) Detection of micrometastasis in sentinel lymph nodes from melanoma patients: direct comparison of multimarker molecular and immunopathological methods Melanoma Res 13:511-520

Ring et al. (1998) Five SWI/SNF-related, matrix-associated, actin-dependent regulator of chromatin (SMARC) genes are dispersed in the human genome Genomics 51:140-143

Ryden et al. (1996) Expression of mRNA for the neurotrophin receptor trkC in neuroblastomas with favourable tumour stage and good prognosis Br. J. Cancer 74:773-779

Saito et al. (2004) Immunohistochemical diagnosis of a rare case of epithelioid malignant peripheral nerve sheath tumor with multiple metastases Jpn. J. Ophthalmol. 48:199-207

Sakwe et al. (2004) Involvement of protein kinase C-alpha and -epsilon in extracellular Ca(2+) signalling mediated by the calcium sensing receptor Exp. Cell Res. 297:560-573

Salazar-Onfray et al. (2002) Tissue distribution and differential expression of melanocortin 1 receptor, a malignant melanoma marker Br. J. Cancer 87:414-422

Salazar-Onfray et al. (2002) Tissue distribution and differential expression of melanocortin 1 receptor, a malignant melanoma marker Br J Cancer 87:414-422

Sasaki et al. (2004) BRAF point mutations in primary melanoma show different prevalences by subtype J. Invest. Dermatol. 123:177-183

Satyamoorthy et al. (2002) A versatile method for the removal of melanin from ribonucleic acids in melanocytic cells Mel. Res. 12:449-452

Schechtman et al. (2004) A critical intramolecular interaction for protein kinase Cepsilon translocation J. Biol. Chem. 279:15831-15840

Schmuth et al. (2004) The effect of LXR activators on AP-I proteins in keratinocytes J. Invest. Dermatol. 123:41-48

Segal et al. (2003) Classification of clear-cell sarcoma as a subtype of melanoma by genomic profiling J. Clin. Oncol. 21:1775-1781

Sells et al. (1997) Human p21-activated kinase (Pakl) regulates actin organization in mammalian cells Curr. Biol. 7:202-210

Seykora et al. (2003) Gene expression profiling of melanocytic lesions Am J Dermatopathol 25:6-11

Shimizu et al. (1999) High expression pf macrophage migration inhibitory factor in human melanoma cells and is role in tumor cell growth and angiogenesis Biochem Biophys Res Commun 264:751-758

Shirotani et al. (2004) Immature nicastrin stabilizes APH-I independent of PEN-2 and presenilin: identification of nicastrin mutants that selectively interact with APH-I J. Neurochem. 89:1520-1527

Shivers et al. (1998) Molecular staging of malignant melanoma: correlation with clinical outcome JAMA 280:1410-1415

Smith et al. (1997) Chromosomal localization of three human dual specificity phosphatase genes (DUSP4, DUSP6, and DUSP7) Genomics 42:524-7

Starz et al. (2003) Tyrosinase RT-PCR as a supplement to histology for detecting melanoma and nevus cells in paraffin sections of sentinel lymph nodes Mod Pathol 16:920-929

Stieber et al. (2003) Molecular basis for the different activation kinetics of the pacemaker channels HCN2 and HCN4 J. Biol. Chem. 278:33672-33680

Stoletov et al. (2004) Nek and Crk mediate distinct VEGF-induced signaling pathways that serve overlapping functions in focal adhesion turnover and integrin activation Exp. Cell Res. 295:258-268

Strausberg et al. (2002) Generation and initial analysis of more than 15,000 full-length human and mouse cDNA sequences Proc. Natl. Acad. Sci. USA 99:16899-16903

Su et al. (2001) Evaluation of acute hepatic failure treated at the Department of Medicine III, Kyushu University Hospital: indications for living-donor liver transplantation Cancer Res. 61:7388-7993

Suzuki et al. (2000) Power spectral analysis of R-R interval variability before and during the sinusoidal heart rate pattern in fetal lambs Int. J. Oncol. 15:1227-1232

Swiatek et al. (2004) Regulation of casein kinase I epsilon activity by Wnt signaling J. Biol. Chem. 279:13011-13017

Szebeni et al. (2003) Role of protein kinase CK2 phosphorylation in the molecular chaperone activity of nucleolar protein b23 J. Biol. Chem. 278:9107-9115

Taback et al. (2000) The clinical utility of multimarker RT-PCR in the detection of occult metastasis in patients with melanoma Recent Results Cancer Res 158:78-92

Takeuchi et al. (2004) Prognostic significance of molecular upstaging of paraffin-embedded sentinel lymph nodes in melanoma patients J Clin Oncol 22:2671-2680

The Washington University-Merck EST Project Hillier et al. (1995)

Thies et al. (2002) Overexpression of the cell adhesion molecule Ll is associated with metastasis in cutaneous malignant melanoma Eur J Cancer 38:1708-1716

Tokuo et al. (2004) Myosin X transports Mena/VASP to the tip of filopodia Biochem. Biophys. Res. Commun. 319:214-220

Tremuth et al. (2004) A fluorescence cell biology approach to map the second integrin-binding site of talin to a 130-amino acid sequence within the rod domain J. Biol. Chem. 279:22258-22266

Tsutsumi et al. (2003) Regulation of cell proliferation by autocrine motility factor/phosphoglucose isomerase signaling J. Biol. Chem. 278:32165-32172

Tusher et al. (2001) Significance analysis of microarrays applied to the ionizing radiation response Proc Natl Acad Sci USA 98:5116-5121

Van Impe et al. (2003) The Nucleo-cytoplasmic actin-binding protein CapG lacks a nuclear export sequence present in structurally related proteins J. Biol. Chem. 278:17945-17952

Vandesompele et al. (2002) Accurate normalization of real-time quantitative RT-PCR data by geometric averaging of multiple internal control genes Genome Biol 3:1-11

Wang et al. (2001) Optimal procedure for extracting RNA from human ocular tissues and expression profiling of the congenital glaucoma gene FOXCl using quantitative RT-PCR MoL Vision 7:89-94

Wang et al. (2002) The third activity for lysyl hydroxylase 3: galactosylation of hydroxylysyl residues in collagens in vitro Matrix Biol. 21:559-566

Wang et al. (2004) Gene expression profiles and molecular markers to predict recurrence of Dukes' B colon cancer J. Clin Oncol 22:1564-1571

Weeraratna (2004) Generation and analysis of melanoma SAGE libraries: SAGE advice on the melanoma transcriptome Oncogene 23:2264-2274

Weiser et al. (2001) Induction of MAGE-3 expression in lung and esophageal cancer cells Ann. Thorac. Surg. 71:295-301

Welk et al. (2001) Identification and characterization of the gene encoding human cytoplasmic polyadenylation element binding protein Gene 263:113-20.

Welsh et al. (2003) Large-scale delineation of secreted protein biomarkers overexpressed in cancer tissue and serum Proc Natl Acad Sci USA 100:3410-3415

Wilson et al. (2000) An early requirement for FGF signaling in the acquisition of neural cell fate in the chick embryo Curr Bio 10:421-429

Woo et al. (2004) Resveratrol inhibits phorbol myristate acetate-induced matrix metalloproteinase-9 expression by inhibiting JNK and PKC delta signal transduction Oncogene 23:1845-1853

Wrightson et al. (2001) Reverse transcriptase-polymerase chain reaction (RT-PCR) analysis of nonsentinel nodes following completion lymphadenectomy for melanoma J. Surg. Res. 98:47-51

Xu et al. (2003) Transforming growth factor-betal stimulated protein kinase B serine-473 and focal adhesion kinase tyrosine phosphorylation dependent on cell adhesion in human hepatocellular carcinoma SMMC-7721 cells Biochem. Biophys. Res. Commun. 312:388-396

Xu et al. (2003) Expression of neurotrophin receptor Trk-C in nevi and melanomas J Cutan Pathol 30:318-322

Yamanaka et al. (1999) Expression of MAGE genes in renal cell carcinoma Int. J. MoI. Med. 2:57-60

Yamauchi et al. (2003) Neurotrophin 3 activation of TrkC induces Schwann cell migration through the c-Jun N-terminal kinase pathway Proc Natl Acad Sci USA 100:14421-14426

Yang et al. (2003) Macrophage inhibitory cytokine-1: a novel biomarker for p53 pathway activation MoI Cancer Therapy 2:1023-1029

Yannoni et al. (2004) P66^{ShcA} interacts with MAPKAP kinase 2 and regulates its activity FEBS Lett. 564:205-211

Yoshimatsu et al. (1998) Expression of the melanoma antigen-encoding gene in human lung cancer J. Surg. Oncol. 67:126-129

Yu et al. (1999) Detection of microscopic melanoma metastases in sentinel lymph nodes Cancer 86:617-627

Yu et al. (2004) A minimal length between tau exon 10 and 11 is required for correct splicing of exon 10 J. Neurochem. 90: 164-172

Zatti et al. (2004) The presenilin 2 M239I mutation associated with familial Alzheimer's disease reduces Ca2+ release from intracellular stores Neurobiol. Dis. 15:269-278

Zhang et al. (2003) Negative regulation of T cell antigen receptor-mediated Crk-L-C3G signaling and cell adhesion by Cbl-b J. Biol. Chem. 278:23978-23983

Zhang et al. (2004) Characterization of the cAMP-dependent protein kinase catalytic subunit Cgamma expressed and purified from sf9 cells Protein Expr. Purif. 35:156-169

Zhang et al. (2004a) Hsp90/p50cdc37 is required for mixed-lineage kinase (MLK) 3 signaling J. Biol. Chem. 279:19457-19463

Zhou et al. (2004) A novel crosstalk mechanism between nuclear receptor-mediated and growth factor/Ras-mediated pathways through PNRC-Grb2 interaction Oncogene 23:5394-5404

1. A method of identifying a melanoma comprising the steps of
a. obtaining a tissue sample; and
b. measuring the expression levels in the sample of genes encoding mRNA corresponding to
PLAB (SEQ ID NO: 1) and LlCAM (SEQ ID NO: 2); or
PLAB, LlCAM and NTRK3 (SEQ ID NO: 3)
wherein the gene expression levels above pre-determined cut-off levels are indicative of the presence of a melanoma in the sample.

2. The method of claim 1 further comprising measuring the expression level of a gene encoding tyrosinase (SEQ ID NO: 999).

3. The method of claim 1 further comprising measuring the expression level of a gene constitutively expressed in the sample.

4. The method of claim 3 wherein the gene encodes PBGD (SEQ ID NOs: 979).

5. The method of claim 1 further comprising measuring the expression levels of at least one gene encoding an mRNA corresponding to a psid selected from the group consisting of SEQ ID NOs: 29-978.

6. The method of claim 1, 2, 3, 4 or 5 wherein the sample is obtained from a lymph node.

7. The method of claim 6 wherein the lymph node is a sentinel lymph node.

8. The method of claim 1, 2, 3, 4 or 5 wherein the sample is obtained from a biopsy.

9. The method of claim 1, 2, 3, 4 or 5 wherein the method is performed intra-operatively.

10. The method of claim 1, 2, 3, 4 or 5 wherein the melanoma is a micrometastasis.

11. The method of claim 1, 2, 3, 4 or 5 wherein the specificity and sensitivity are sufficient to detect metastasis of melanoma.

12. The method of claim 11, wherein the specificity is at least 95% based on a comparison of hematoxylin and eosin (H&E) and immunohistochemical (IHC) negative nodes.

13. The method of claim 11 wherein the specificity is at least 97% based on a comparison of H&E and IHC negative nodes.

14. The method of claim 11 wherein the specificity is at least 99% based on a comparison of H&E and IHC negative nodes.

15. The method of claim 11 wherein the sensitivity is at least at least 80% based on a comparison of hematoxylin and eosin (H&E) and immunohistochemical (IHC) positive nodes.

16. The method of claim 11 wherein the sensitivity is at least 85% based on a comparison of H&E and IHC positive nodes.

17. The method of claim 11 wherein the sensitivity is at least 90% based on a comparison of H&E and IHC positive nodes.

18. The method of claim 11 wherein the specificity and sensitivity are at least 97% based on a comparison of H&E and IHC negative nodes and at least 85% based on a comparison of H&E and IHC positive nodes, respectively.

19. The method of claim 1, 2, 3, 4 or 5 wherein the pre-determined cut-off levels are at least two-fold over-expression in tissue having metastatic melanoma relative to benign melanocyte or normal tissue.

20. The method of claim 1, 2, 3, 4 or 5 wherein gene expression is measured on a microarray or gene chip.

21. The method of claim 1, 2, 3, 4 or 5 wherein gene expression is determined by nucleic acid amplification conducted by polymerase chain reaction (PCR) of RNA extracted from the sample.

22. The method of claim 21 wherein the PCR products comprise at least one of SEQ ID NOs: 25-28.

23. The method of claim 22 wherein the PCR products include fluorophores.

24. The method of claim 23 wherein the fluorophores are selected from the group consisting of Fam, Texas Red, CaI Red, Cl, CyS, and Cy3.

25. The method of claim 24 wherein the fluorophores correspond to PLAB: Fam; LlCAM: Texas Red or CaI Red, tyrosinase: Cl; PBGD: Cy5, where applicable.

26. The method of claim 21 wherein said PCR is reverse transcription polymerase chain reaction (RT-PCR).

27. The method of claim 26, wherein the RT-PCR further comprises one or more internal control reagents.

28. The method of claim 21 wherein RNA is extracted from the sample by:
a. homogenizing the sample to produce an homogenate;
b. contacting the homogenate with a substrate containing, or to which is affixed, an RNA-binding material;
c. allowing the RNA to bind to the RNA binding material;
d. washing the substrate under conditions sufficient to remove any contaminants, interferents and un-bound RNA; and
e. eluting bound RNA from the substrate.

29. The method of claim 1, 2, 3, 4 or 5 further comprising reducing melanin in the sample.

30. The method of claim 29 wherein melanin concentration is reduced by homogenizing the sample to produce an homogenate and passing the homogenate through a matrix to which melanin adheres, bonds, or is affixed.

31. The method of claim 30 wherein said matrix comprises polymeric beads.

32. The method of claim 30 wherein said matrix comprises silica.

33. The method of claim 28 wherein the RNA is extracted in less than about 8 minutes.

34. The method of claim 28 wherein the RNA is extracted in less than about 6 minutes.

35. The method of claim 1, 2, 3, 4 or 5 wherein gene expression is measured by measuring the protein encoded by the gene.

36. The method of claim 35 wherein the protein is detected by an antibody specific to the protein.

37. A method of identifying a melanoma comprising the steps of
a. obtaining a tissue sample; and
b. measuring the expression levels in the sample of genes encoding niRNA recognized by the primer/probe sets selected from the group consisting of SEQ ID NOs: 4-6 or SEQ ID NOs: 7-9 and SEQ ID NOs: 10-12 or SEQ ID NOs: 13-15; or
SEQ ID NOs: 4-6 or SEQ ID NOs: 7-9 and SEQ ID NOs: 10-12 or SEQ ID NOs: 13-15 and SEQ ID NOs: 16-18
wherein the gene expression levels above pre-determined cut-off levels are indicative of the presence of a melanoma in the sample.

38. The method of claim 37 further comprising measuring the expression level of a gene encoding tyrosinase (SEQ ID NO: 999).

39. The method of claim 37 further comprising measuring the expression level of a gene constitutively expressed in the sample.

40. The method of claim 39 wherein the gene encodes PBGD (SEQ ID NO: 979).

41. The method of claim 37 further comprising measuring the expression levels of at least one gene encoding an mRNA corresponding to a psid selected from the group consisting of SEQ ID NOs: 29-978.

42. The method of claim 37, 38, 39, 40 or 41 wherein the sample is obtained from a lymph node.

43. The method of claim 42 wherein the lymph node is a sentinel lymph node.

44. The method of claim 37, 38, 39, 40 or 41 wherein the sample is obtained from a biopsy.

45. The method of claim 37, 38, 39, 40 or 41 wherein the method is performed intra-operatively.

46. The method of claim 37, 38, 39, 40 or 41 wherein the melanoma is a micrometastasis.

47. The method of claim 37, 38, 39, 40 or 41 wherein the specificity and sensitivity are sufficient to detect metastasis of melanoma.

48. The method of claim 47, wherein the specificity is at least 95% based on a comparison of hematoxylin and eosin (H&E) and immunohistochemical (IHC) negative nodes.

49. The method of claim 47 wherein the specificity is at least 97% based on a comparison of H&E and IHC negative nodes.

50. The method of claim 47 wherein the specificity is at least 99% based on a comparison of H&E and IHC negative nodes.

51. The method of claim 47 wherein the sensitivity is at least at least 80% based on a comparison of hematoxylin and eosin (H&E) and immunohistochemical (IHC) positive nodes.

52. The method of claim 47 wherein the sensitivity is at least 85% based on a comparison of H&E and IHC positive nodes.

53. The method of claim 47 wherein the sensitivity is at least 90% based on a comparison of H&E and IHC positive nodes.

54. The method of claim 47 wherein the specificity and sensitivity are at least 97% based on a comparison of H&E and IHC negative nodes and at least 85% based on a comparison of H&E and IHC positive nodes, respectively.

55. The method of claim 37, 38, 39, 40 or 41 wherein the pre-determined cut-off levels are at least two-fold over-expression in tissue having metastatic melanoma relative to benign melanocyte or normal tissue.

56. The method of claim 37, 38, 39, 40 or 41 wherein gene expression is measured on a microarray or gene chip.

57. The method of claim 37, 38, 39, 40 or 41 wherein gene expression is determined by nucleic acid amplification conducted by polymerase chain reaction (PCR) of RNA extracted from the sample.

58. The method of claim 57 wherein the PCR products comprise at least one of SEQ ID NOs: 25-28.

59. The method of claim 57 wherein the PCR products include fluorophores.

60. The method of claim 59 wherein the fluorophores are selected from the group consisting of Fam, Texas Red, CaI Red, Cl, Cy5 and Cy3.

61. The method of claim 60 wherein the fluorophores correspond to PLAB: Fam; LlCAM: Texas Red or CaI Red, tyrosinase: Cl; PBGD: Cy5, where applicable.

62. The method of claim 57 wherein said PCR is reverse transcription polymerase chain reaction (RT-PCR).

63. The method of claim 62, wherein the RT-PCR further comprises one or more internal control reagents.

64. The method of claim 57 wherein RNA is extracted from the sample by:
a. homogenizing the sample to produce an homogenate;
b. contacting the homogenate with a substrate containing, or to which is affixed, an RNA-binding material;
c. allowing the RNA to bind to the RNA binding material;
d. washing the substrate under conditions sufficient to remove any contaminants, interferents and un-bound RNA; and
e. eluting bound RNA from the substrate.

65. The method of claim 37, 38, 39, 40 or 41 further comprising reducing melanin in the sample.

66. The method of claim 65 wherein melanin concentration is reduced by homogenizing the sample to produce an homogenate and passing the homogenate through a matrix to which melanin adheres, bonds, or is affixed.

67. The method of claim 66 wherein the matrix comprises polymeric beads.

68. The method of claim 66 wherein the matrix comprises silica.

69. The method of claim 64 wherein the RNA is extracted in less than about 8 minutes.

70. The method of claim 64 wherein the RNA is extracted in less than about 6 minutes.

71. The method of claim 37, 38, 39, 40 or 41 wherein gene expression is measured by measuring the protein encoded by the gene.

72. The method of claim 71 wherein the protein is detected by an antibody specific to the protein.

73. A method of distinguishing a malignant melanocyte from a benign melanocyte comprising the steps of
a. obtaining a tissue sample; and
b. measuring the expression levels in the sample of genes encoding
PLAB (SEQ ID NO: 1) and LlCAM (SEQ ID NO: 2); or
PLAB, LlCAM and NTRK3 (SEQ ID NO: 3)
wherein the gene expression levels above pre-determined cut-off levels are indicative of the presence of a malignant melanocyte in the sample.

74. The method of claim 73 further comprising measuring the expression level of a gene encoding tyrosinase (SEQ ID NO: 999).

75. The method of claim 73 further comprising measuring the expression level of a gene constitutively expressed in the sample.

76. The method of claim 75 wherein the gene encodes PBGD (SEQ ID NO: 979).

77. The method of claim 73 further comprising measuring the expression levels of at least one gene encoding an mRNA corresponding to a psid selected from the group consisting of SEQ ID NOs: 29-978.

78. The method of claim 73, 74, 75, 76 or 77 wherein the sample is obtained from a lymph node.

79. The method of claim 78 wherein the lymph node is a sentinel lymph node.

80. The method of claim 73, 74, 75, 76 or 77 wherein the sample is obtained from a biopsy.

81. The method of claim 73, 74, 75, 76 or 77 wherein the method is performed intra-operatively.

82. The method of claim 73, 74, 75, 76 or 77 wherein the melanoma is a micrometastasis.

83. The method of claim 73, 74, 75, 76 or 77 wherein the specificity and sensitivity are sufficient to detect metastasis of melanoma.

84. The method of claim 83, wherein the specificity is at least 95% based on a comparison of hematoxylin and eosin (H&E) and immunohistochemical (IHC) negative nodes.

85. The method of claim 83 wherein the specificity is at least 97% based on a comparison of H&E and IHC negative nodes.

86. The method of claim 83 wherein the specificity is at least 99% based on a comparison of H&E and IHC negative nodes.

87. The method of claim 83 wherein the sensitivity is at least at least 80% based on a comparison of hematoxylin and eosin (H&E) and immunohistochemical (IHC) positive nodes.

88. The method of claim 83 wherein the sensitivity is at least 85% based on a comparison of H&E and IHC positive nodes.

89. The method of claim 83 wherein the sensitivity is at least 90% based on a comparison of H&E and IHC positive nodes.

90. The method of claim 83 wherein the specificity and sensitivity are at least 97% based on a comparison of H&E and IHC negative nodes and at least 85% based on a comparison of H&E and IHC positive nodes, respectively.

91. The method of claim 73, 74, 75, 76 or 77 wherein the pre-determined cut-off levels are at least two-fold over-expression in tissue having metastatic melanoma relative to benign melanocyte or normal tissue.

92. The method of claim 73, 74, 75, 76 or 77 wherein gene expression is measured on a microarray or gene chip.

93. The method of claim 73, 74, 75, 76 or 77 wherein gene expression is determined by nucleic acid amplification conducted by polymerase chain reaction (PCR) of RNA extracted from the sample.

94. The method of claim 93 wherein the PCR products comprise at least one of SEQ ID NOs: 25-28.

95. The method of claim 93 wherein the PCR products include fluorophores.

96. The method of claim 95 wherein the fluorophores are selected from the group consisting of Fam, Texas Red, CaI Red, Cl, Cy5 and Cy3.

97. The method of claim 96 wherein the PCR product, if present, is identified by the fluorescence pattern of PLAB: Fam; LlCAM: Texas Red or CaI Red, tyrosinase: Cl; PBGD: Cy5, where applicable.

98. The method of claim 93 wherein the PCR is reverse transcription polymerase chain reaction (RT-PCR).

99. The method of claim 98, wherein the RT-PCR further comprises one or more internal control reagents.

100. The method of claim 93 wherein RNA is extracted from the sample by:
a. homogenizing the sample to produce an homogenate;
b. contacting the homogenate with a substrate containing, or to which is affixed, an RNA-binding material;
c. allowing the RNA to bind to the RNA binding material;
d. washing the substrate under conditions sufficient to remove any contaminants, interferents and un-bound RNA; and
e. eluting bound RNA from the substrate.

101. The method of claim 73, 74, 75, 76 or 77 further comprising reducing melanin in the sample.

102. The method of claim 101 wherein melanin concentration is reduced by homogenizing the sample to produce an homogenate and passing the homogenate through a matrix to which melanin adheres, bonds, or is affixed.

103. The method of claim 102 wherein the matrix comprises polymeric beads.

104. The method of claim 102 wherein the matrix comprises silica.

105. The method of claim 100 wherein the RNA is extracted in less than about 8 minutes.

106. The method of claim 100 wherein the RNA is extracted in less than about 6 minutes.

107. The method of claim 73, 74, 75, 76 or 77 wherein gene expression is measured by measuring the protein encoded by the gene.

108. The method of claim 107 wherein the protein is detected by an antibody specific to the protein.

109. A method of distinguishing a malignant melanocyte from a benign melanocyte comprising the steps of
a. obtaining a tissue sample; and
b. measuring the expression levels in the sample of genes recognized by the primer/probe sets selected from the group consisting of
SEQ ID NOs: 4-6 or SEQ ID NOs: 7-9 and SEQ ID NOs: 10-12 or SEQ ID NOs: 13-15; or
SEQ ID NOs: 4-6 or SEQ ID NOs: 7-9 and SEQ ID NOs: 10-12 or SEQ ID NOs: 13-15 and SEQ ID NOs: 16-18
wherein the gene expression levels above pre-determined cut-off levels are indicative of the presence of a malignant melanocyte in the sample.

110. The method of claim 109 further comprising measuring the expression level of a gene encoding tyrosinase (SEQ ID NO: 999).

111. The method of claim 109 further comprising measuring the expression level of a gene constitutively expressed in the sample.

112. The method of claim 110 wherein the gene encodes PBGD (SEQ ID NO: 979).

113. The method of claim 109 farther comprising measuring the expression levels of at least one gene encoding an mRNA corresponding to a psid selected from the group consisting of SEQ ID NOs: 29-978.

114. The method of claim 109, 110, 111, 112 or 113 wherein the sample is obtained from a lymph node.

115. The method of claim 114 wherein the lymph node is a sentinel lymph node.

116. The method of claim 109, 110, 111, 112 or 113 wherein the sample is obtained from a biopsy.

117. The method of claim 109, 110, 111, 112 or 113 wherein the method is performed intra-operatively.

118. The method of claim 109, 110, 111, 112 or 113 wherein the melanoma is a micrometastasis.

119. The method of claim 109, 110, 111, 112 or 113 wherein the specificity and sensitivity are sufficient to detect metastasis of melanoma.

120. The method of claim 119, wherein the specificity is at least 95% based on a comparison of hematoxylin and eosin (H&E) and immunohistochemical (IHC) negative nodes.

121. The method of claim 119 wherein the specificity is at least 97% based on a comparison of H&E and IHC negative nodes.

122. The method of claim 119 wherein the specificity is at least 99% based on a comparison of H&E and IHC negative nodes.

123. The method of claim 119 wherein the sensitivity is at least at least 80% based on a comparison of hematoxylin and eosin (H&E) and immunohistochemical (IHC) positive nodes.

124. The method of claim 119 wherein the sensitivity is at least 85% based on a comparison of H&E and IHC positive nodes.

125. The method of claim 119 wherein the sensitivity is at least 90% based on a comparison of H&E and IHC positive nodes.

126. The method of claim 119 wherein the specificity and sensitivity are at least 97% based on a comparison of H&E and IHC negative nodes and at least 85% based on a comparison of H&E and IHC positive nodes, respectively.

127. The method of claim 109, 110, 111, 112 or 113 wherein the pre-determined cut-off levels are at least two-fold over-expression in tissue having metastatic melanoma relative to benign melanocyte or normal tissue.

128. The method of claim 109, 110, 111, 112 or 113 wherein gene expression is measured on a microarray or gene chip.

129. The method of claim 109, 110, 111, 112 or 113 wherein gene expression is determined by nucleic acid amplification conducted by polymerase chain reaction (PCR) of RNA extracted from the sample.

130. The method of claim 129 wherein the PCR products comprise at least one of SEQ ID NOs: 25-28.

131. The method of claim 129 wherein the PCR products include fluorophores.

132. The method of claim 131 wherein the fluorophores are selected from the group consisting of Fam, Texas Red, CaI Red, Cl, Cy5 and Cy3.

133. The method of claim 132 wherein the PCR product, if present, is identified by the fluorescence pattern of PLAB: Fam; LlCAM: Texas Red or CaI Red, tyrosinase: Cl; PBGD: Cy5, where applicable.

134. The method of claim 128 wherein the PCR is reverse transcription polymerase chain reaction (RT-PCR).

135. The method of claim 134, wherein the RT-PCR further comprises one or more internal control reagents.

136. The method of claim 129 wherein RNA is extracted from the sample by:
a. homogenizing the sample to produce an homogenate;
b. contacting the homogenate with a substrate containing, or to which is affixed, an RNA-binding material;
c. allowing the RNA to bind to the RNA binding material;
d. washing the substrate under conditions sufficient to remove any contaminants, interferents and un-bound RNA; and
e. eluting bound RNA from the substrate.

137. The method of claim 109, 110, 111; 112 or 113 further comprising reducing melanin in the sample.

138. The method of claim 136 wherein melanin concentration is reduced by homogenizing the sample to produce an homogenate and passing the homogenate through a matrix to which melanin adheres, bonds, or is affixed.

139. The method of claim 136 wherein the matrix comprises polymeric beads.

140. The method of claim 136 wherein the matrix comprises silica.

141. The method of claim 136 wherein the RNA is extracted in less than about 8 minutes.

142. The method of claim 136 wherein the RNA is extracted in less than about 6 minutes.

143. The method of claim 109, 110, 111, 112 or 113 wherein gene expression is measured by measuring the protein encoded by the gene.

144. The method of claim 143 wherein the protein is detected by an antibody specific to the protein.

145. A method of determining patient treatment protocol comprising the steps of
a. obtaining a tissue sample from the patient; and
b. measuring the expression levels in the sample of genes encoding PLAB (SEQ ID NO:1) and LlCAM (SEQ ID NO:2); or
PLAB, LlCAM and NTRK3 (SEQ ID NO:3)
wherein the gene expression levels above pre-determined cut-off levels are indicative of the presence of a melanoma in the sample.

146. The method of claim 145 further comprising measuring the expression level of a gene encoding tyrosinase (SEQ ID NO: 999).

147. The method of claim 145 further comprising measuring the expression level of a gene constitutively expressed in the sample.

148. The method of claim 147 wherein the gene encodes PBGD (SEQ ID NO: 979).

149. The method of claim 145 further comprising measuring the expression levels of at least one gene encoding an mRNA corresponds to a psid selected from the group consisting of SEQ ID NOs: 29-978.

150. The method of claim 145, 146, 147, 148 or 149 wherein the sample is obtained from a lymph node.

151. The method of claim 150 wherein the lymph node is a sentinel lymph node.

152. The method of claim 145, 146, 147, 148 or 149 wherein the sample is obtained from a biopsy.

153. The method of claim 145, 146, 147, 148 or 149 wherein the method is performed intra-operatively.

154. The method of claim 145, 146, 147, 148 or 149 wherein the melanoma is a micrometastasis.

155. The method of claim 145, 146, 147, 148 or 149 wherein the specificity and sensitivity are sufficient to detect metastasis of melanoma.

156. The method of claim 155, wherein the specificity is at least 95% based on a comparison of hematoxylin and eosin (H&E) and immunohistochemical (IHC) negative nodes.

157. The method of claim 155 wherein the specificity is at least 97% based on a comparison of H&E and IHC negative nodes.

158. The method of claim 155 wherein the specificity is at least 99% based on a comparison of H&E and IHC negative nodes.

159. The method of claim 155 wherein the sensitivity is at least at least 80% based on a comparison of hematoxylin and eosin (H&E) and immunohistochemical (IHC) positive nodes.

160. The method of claim 155 wherein the sensitivity is at least 85% based on a comparison of H&E and IHC positive nodes.

161. The method of claim 155 wherein the sensitivity is at least 90% based on a comparison of H&E and IHC positive nodes.

162. The method of claim 155 wherein the specificity and sensitivity are at least 97% based on a comparison of H&E and IHC negative nodes and at least 85% based on a comparison of H&E and IHC positive nodes, respectively.

163. The method of claim 145, 146, 147, 148 or 149 wherein the pre-determined cut-off levels are at least two-fold over-expression in tissue having metastatic melanoma relative to benign melanocyte or normal tissue.

164. The method of claim 145, 146, 147, 148 or 149 wherein gene expression is measured on a microarray or gene chip.

165. The method of claim 145, 146, 147, 148 or 149 wherein gene expression is determined by nucleic acid amplification conducted by polymerase chain reaction (PCR) of RNA extracted from the sample.

166. The method of claim 165 wherein the PCR products comprise at least one of SEQ ID NOs: 25-28.

167. The method of claim 165 wherein the PCR products include fluorophores.

168. The method of claim 167 wherein the fluorophores are selected from the group consisting of Fam, Texas Red, CaI Red, Cl, Cy5 and Cy3.

169. The method of claim 168 wherein the Fluorophores correspond to PLAB: Fam; LlCAM: Texas Red or CaI Red, tyrosinase: Cl; PBGD: Cy5, where applicable.

170. The method of claim 152 wherein the PCR is reverse transcription polymerase chain reaction (RT-PCR).

171. The method of claim 170, wherein the RT-PCR further comprises one or more internal control reagents.

172. The method of claim 165 wherein RNA is extracted from the sample by:
a. homogenizing the sample to produce an homogenate;
b. contacting the homogenate with a substrate containing, or to which is affixed, an RNA-binding material;
c. allowing the RNA to bind to the RNA binding material;
d. washing the substrate under conditions sufficient to remove any contaminants, interferents and un-bound RNA; and
e. eluting bound RNA from the substrate.

173. The method of claim 145, 146, 147, 148 or 149 further comprising reducing melanin in the sample.

174. The method of claim 173 wherein melanin concentration is reduced by homogenizing the sample to produce an homogenate and passing the homogenate through a matrix to which melanin adheres, bonds, or is affixed.

175. The method of claim 174 wherein the matrix comprises polymeric beads.

176. The method of claim 174 wherein the matrix comprises silica.

177. The method of claim 172 wherein the RNA is extracted in less than about 8 minutes.

178. The method of claim 172 wherein the RNA is extracted in less than about 6 minutes.

179. The method of claim 145, 146, 147, 148 or 149 wherein gene expression is measured by measuring the protein encoded by the gene.

180. The method of claim 179 wherein the protein is detected by an antibody specific to the protein.

181. A method of determining patient treatment protocol comprising the steps of
a. obtaining a tissue sample from the patient; and
b. measuring the expression levels in the sample of genes identified by the primer/probe sets selected from the group consisting of
SEQ ID NOs: 4-6 or SEQ ID NOs: 7-9 and SEQ ID NOs: 10-12 or SEQ ID NOs: 13-15; or
SEQ ID NOs: 4-6 or SEQ ID NOs: 7-9 and SEQ ID NOs: 10-12 or SEQ ID NOs: 13-15 and SEQ ID NOs: 16-18
wherein the gene expression levels above pre-determined cut-off levels are indicative of the presence of a melanoma in the sample.

182. The method of claim 181 further comprising measuring the expression level of a gene encoding tyrosinase (SEQ ID NO: 999).

183. The method of claim 181 further comprising measuring the expression level of a gene constitutively expressed in the sample.

184. The method of claim 181 wherein the gene encodes PBGD (SEQ ID NO: 979).

185. The method of claim 184 further comprising measuring the expression levels of at least one gene encoding an mRNA correspond to a psid selected from the group consisting of SEQ ID NOs: 29-978.

186. The method of claim 181, 182, 183, 184 or 185 wherein the sample is obtained from a lymph node.

187. The method of claim 186 wherein the lymph node is a sentinel lymph node.

188. The method of claim 181, 182, 183, 184 or 185 wherein the sample is obtained from a biopsy.

189. The method of claim 181, 182, 183, 184 or 185 wherein the method is performed intra-operatively.

190. The method of claim 181, 182, 183, 184 or 185 wherein the melanoma is a micrometastasis.

191. The method of claim 181, 182, 183, 184 or 185 wherein the specificity and sensitivity are sufficient to detect metastasis of melanoma.

192. The method of claim 191, wherein the specificity is at least 95% based on a comparison of hematoxylin and eosin (H&E) and immunohistochemical (IHC) negative nodes.

193. The method of claim 191 wherein the specificity is at least 97% based on a comparison of H&E and IHC negative nodes.

194. The method of claim 191 wherein the specificity is at least 99% based on a comparison of H&E and IHC negative nodes.

195. The method of claim 191 wherein the sensitivity is at least at least 80% based on a comparison of hematoxylin and eosin (H&E) and immunohistochemical (IHC) positive nodes.

196. The method of claim 191 wherein the sensitivity is at least 85% based on a comparison of H&E and IHC positive nodes.

197. The method of claim 191 wherein the sensitivity is at least 90% based on a comparison of H&E and IHC positive nodes.

198. The method of claim 191 wherein the specificity and sensitivity are at least 97% based on a comparison of H&E and IHC negative nodes and at least 85% based on a comparison of H&E and IHC positive nodes, respectively.

199. The method of claim 181, 182, 183, 184 or 185 wherein the pre-determined cut-off levels are at least two-fold over-expression in tissue having metastatic melanoma relative to benign melanocyte or normal tissue.

200. The method of claim 181, 182, 183, 184 or 185 wherein gene expression is measured on a microarray or gene chip.

201. The method of claim 181, 182, 183, 184 or 185 wherein gene expression is determined by nucleic acid amplification conducted by polymerase chain reaction (PCR) of RNA extracted from the sample.

202. The method of claim 201 wherein the PCR products comprise at least one of SEQ ID NOs: 25-28.

203. The method of claim 201 wherein the PCR products include fluorophores.

204. The method of claim 203 wherein the fluorophores are selected from the group consisting of Fam, Texas Red, CaI Red, Cl, Cy5 and Cy3.

205. The method of claim 204 wherein the Fluorophores correspond to PLAB: Fam; LlCAM: Texas Red or CaI Red, tyrosinase: Cl; PBGD: Cy5, where applicable.

206. The method of claim 201 wherein the PCR is reverse transcription polymerase chain reaction (RT-PCR).

207. The method of claim 206, wherein the RT-PCR further comprises one or more internal control reagents.

208. The method of claim 201 wherein RNA is extracted from the sample by:
a. homogenizing the sample to produce an homogenate;
b. contacting the homogenate with a substrate containing, or to which is affixed, an RNA-binding material;
c. allowing the RNA to bind to the RNA binding material;
d. washing the substrate under conditions sufficient to remove any contaminants, interferents and un-bound RNA; and
e. eluting bound RNA from the substrate.

209. The method of claim 181, 182, 183, 184 or 185 further comprising reducing melanin in the sample.

210. The method of claim 209 wherein melanin concentration is reduced by homogenizing the sample to produce an homogenate and passing the homogenate through a matrix to which melanin adheres, bonds, or is affixed.

211. The method of claim 210 wherein the matrix comprises polymeric beads.

212. The method of claim 210 wherein the matrix comprises silica.

213. The method of claim 208 wherein the RNA is extracted in less than about 8 minutes.

214. The method of claim 208 wherein the RNA is extracted in less than about 6 minutes.

215. The method of claim 181, 182, 183, 184 or 185 wherein gene expression is measured by measuring the protein encoded by the gene.

216. The method of claim 215 wherein the protein is detected by an antibody specific to the protein.

217. A composition comprising at least one primer/probe set selected from the group consisting of: SEQ ID NOs: 4-6, SEQ ID NOs: 7-9, SEQ ID NO:46-48, SEQ ID NOs: 13-15, SEQ ID NOs: 16-18, SEQ ID NOs: 19-21, and SEQ ID NOs: 22-24.

218. A composition comprising at least one amplicon selected from the group consisting of SEQ ID NOs: 25-28.

219. A kit for conducting an assay to determine the presence of melanoma in a tissue sample comprising: nucleic acid amplification and detection reagents.

220. The kit of claim 219 wherein the reagents comprise primers having sequences for detecting the expression of at least one gene encoding an mRNA selected from the group consisting of SEQ ID NOs: 1-3.

221. The kit of claim 219 comprising RT-PCR reagents.
